(19) United States
(12) Patent Application Publication
(10) Pub. No.: US 2020/0062773 A1
(43) Pub. Date: Feb. 27, 2020

(54) 4,4a,5,7-TETRAHYDRO-3H-FURO[3,4-b]PYRIDINYL COMPOUNDS

(71) Applicant: Janssen Pharmaceutlca NV, Beerse (BE)

(72) Inventors: Sven Franciscus Anna Van Brandt, Beerse (BE); Gijsen Henricus Jacobus Maria, Breda (NL); Frederik Jari Rita Rombouts, Wilrijk (BE)

(73) Assignee: Janssen Pharmaceutica NV, Beerse (BE)

(21) Appl. No.: 16/347,064

(22) PCT Filed: Nov. 3, 2017
(86) PCT No.: PCT/EP2017/078200

§ 371 Date: May 2, 2019

(30) Foreign Application Priority Data
Nov. 4, 2016 (EP) 16197351.6
Mar. 22, 2017 (EP) 17162307.7

(51) Int. Cl. C07D 491/048; A61P 25/28

(52) U.S. Cl. CPC C07D 491/048; A61P 25/28

(57) ABSTRACT

The present invention relates to 4,4a,5,7-tetrahydro-3H-furo[3,4-b]pyridinyl compound inhibitors of beta-site APP-cleaving enzyme having the structure shown in Formula (I)

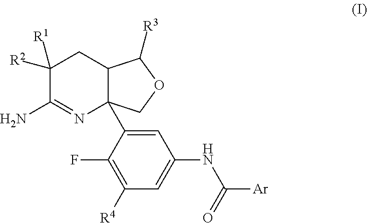

wherein the radicals are as defined in the specification. The invention is also directed to pharmaceutical compositions comprising such compounds, to processes for preparing such compounds and compositions, and to the use of such compounds and compositions for the prevention and treatment of disorders in which beta-site APP-cleaving enzyme is involved, such as Alzheimer's disease (AD), mild cognitive impairment, preclinical Alzheimer's disease, senility, dementia, dementia with Lewy bodies, Down's syndrome, dementia associated with stroke, dementia associated with Parkinson's disease, and dementia associated with beta-amyloid.

### FIELD OF THE INVENTION

The present invention relates to 4,4a,5,7-tetrahydro-3H-furo[3,4-b]pyridinyl compound inhibitors of beta-site APP-cleaving enzyme having the structure shown in Formula (I)

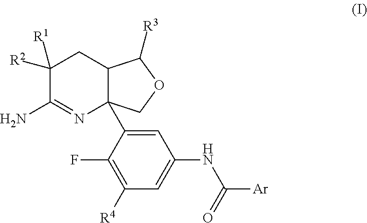

wherein the radicals are as defined in the specification. The invention is also directed to pharmaceutical compositions comprising such compounds, to processes for preparing such compounds and compositions, and to the use of such compounds and compositions for the prevention and treatment of disorders in which beta-site APP-cleaving enzyme is involved, such as Alzheimer's disease (AD), mild cognitive impairment, preclinical Alzheimer's disease, senility, dementia, dementia with Lewy bodies, Down's syndrome, dementia associated with stroke, dementia associated with Parkinson's disease, and dementia associated with beta-amyloid.

### BACKGROUND OF THE INVENTION

Alzheimer's Disease (AD) is a neurodegenerative disease associated with aging. AD patients suffer from cognition deficits and memory loss as well as behavioral problems such as anxiety. Over 90% of those afflicted with AD have a sporadic form of the disorder while less than 10% of the cases are familial or hereditary. In the United States, about one in ten people at age 65 have AD while at age 85, one out of every two individuals are afflicted by AD. The average life expectancy from the initial diagnosis is 7-10 years, and AD patients require extensive care either in an assisted living facility or by family members. With the increasing number of elderly in the population, AD is a growing medical concern. Currently available therapies for AD merely treat the symptoms of the disease and include acetylcholinesterase inhibitors to improve cognitive properties as well as anxiolytics and antipsychotics to control the behavioral problems associated with this ailment.

The hallmark pathological features in the brain of AD patients are neurofibrillary tangles which are generated by hyperphosphorylation of tau protein and amyloid plaques which form by aggregation of beta-amyloid 1-42 (Abeta 1-42) peptide. Abeta 1-42 forms oligomers and then fibrils, and ultimately amyloid plaques. The oligomers and fibrils are believed to be especially neurotoxic and may cause most of the neurological damage associated with AD. Agents that prevent the formation of Abeta 1-42 have the potential to be disease-modifying agents for the treatment of AD. Abeta 1-42 is generated from the amyloid precursor protein (APP), comprised of 770 amino acids. The N-terminus of Abeta 1-42 is cleaved by beta-site APP-cleaving enzyme (BACE1), and then gamma-secretase cleaves the C-terminal end. In addition to Abeta 1-42, gamma-secretase also liberates Abeta 1-40 which is the predominant cleavage product as well as Abeta 1-38 and Abeta 1-43. These Abeta forms can also aggregate to form oligomers and fibrils. Thus, inhibitors of BACE1 would be expected to prevent the formation of Abeta 1-42 as well as Abeta 1-40, Abeta 1-38 and Abeta 1-43 and would be potential therapeutic agents in the treatment of AD.

US2011/009395 (Audia James Edmund) discloses 4a,5,7,7a-tetrahydro-4H-furo[3,4-d][1,3]thiazin-2-amine derivatives, in particular LY2886721 which was in a Phase 2 trial until June 2013 when its development was terminated due to liver abnormalities that showed up in four out of 45 patients. WO2014/099794 (Merck Sharp & Dohme) discloses 1,1-dioxo-4a,5,7,7a-tetrahydro-2H-furo[3,4-b][1,4]thiazin-3-amine derivatives; WO2016/096979 (Janssen Pharmaceutica NV) discloses 4-(trifluoromethyl)-2,3,4,5-tetrahydropyridin-6-amine derivatives as BACE inhibitors; and Bioorg. Med. Chem. Lett. 2014, 24(9), 2033-2045 reviews amidine-based BACE inhibitors.

There is still a need for therapies against Alzheimer's disease and other neurodegenerative diseases, in particular by making available alternative compounds with an advantageous balance of properties, e.g. compounds that are devoid of reactive metabolites, that do not induce adverse reactions such as liver abnormalities, and that inhibit beta-site APP cleaving enzyme 2 (BACE2) to a lesser extent than the intended target beta-site APP-cleaving enzyme 1 (BACE1).

### SUMMARY OF THE INVENTION

The present invention is directed to compounds of Formula (I)

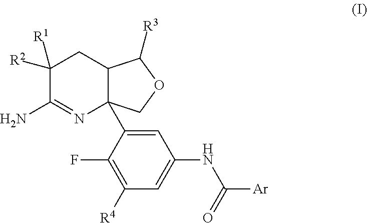

and the tautomers and the stereoisomeric forms thereof, wherein
R¹ is selected from the group consisting of hydrogen, C₁₋₄alkyl, monohalo-C₁₋₄alkyl, and polyhalo-C₁₋₄alkyl;
R² is selected from the group consisting of hydrogen, cyano, C₁₋₄alkyloxy, —SO₂C₁₋₄alkyl, —SO₂cyclopropyl, and —SO(NCH₃)CH₃;
R³ is selected from the group consisting of hydrogen, C₁₋₄alkyl optionally substituted with 1, 2 or 3 fluoro substituents, and cyclopropyl optionally substituted with 1 or 2 fluoro substituents;
R⁴ is hydrogen or fluoro;
Ar is homoaryl or heteroaryl;
wherein homoaryl is phenyl, or phenyl substituted with one, two or three substituents each independently selected from the group consisting of halo, cyano, C₁₋₄alkyl, C₁₋₄alkyloxy, monohalo-C₁₋₄alkyl, polyhalo-C₁₋₄alkyl, monohalo-C₁₋₄alkyloxy, and polyhalo-C₁₋₄alkyloxy;
heteroaryl is selected from the group consisting of pyridyl, pyrimidinyl, pyrazinyl, pyridazinyl, furanyl, thienyl, pyrrolyl, pyrazolyl, imidazolyl, triazolyl, tetrazolyl, thiazolyl, isothiazolyl, thiadiazolyl, oxazolyl, isoxazolyl, and oxadiazolyl, each optionally substituted with one, two or three substituents each independently selected from the group consisting of halo, cyano, C₁₋₄alkyl, C₂₋₄alkynyl, C₁₋₄alkyloxy, monohalo-C₁₋₄alkyl, polyhalo-C₁₋₄alkyl, monohalo-C₁₋₄alkyloxy,
polyhalo-C₁₋₄alkyloxy, C₁₋₄alkyloxyC₁₋₄alkyloxy and triazolyl, in particular 1,2,4-triazol-1-yl;
and the pharmaceutically acceptable acid addition salts thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of treating a disorder mediated by the beta-site APP-cleaving enzyme, comprising administering to a subject in need thereof a therapeutically effective amount of any of the compounds or pharmaceutical compositions described above.

Further exemplifying the invention are methods of inhibiting the beta-site APP-cleaving enzyme, comprising administering to a subject in need thereof a therapeutically effective amount of any of the compounds or pharmaceutical compositions described above.

An example of the invention is a method of treating a disorder selected from the group consisting of Alzheimer's disease, mild cognitive impairment, preclinical Alzheimer's disease, senility, dementia, dementia with Lewy bodies, Down's syndrome, dementia associated with stroke, dementia associated with Parkinson's disease, and dementia associated with beta-amyloid, preferably Alzheimer's disease, comprising administering to a subject in need thereof, a therapeutically effective amount of any of the compounds or pharmaceutical compositions described above.

Another example of the invention is any of the compounds described above for use in treating: (a) Alzheimer's Disease, (b) mild cognitive impairment, (c) senility, (d) dementia, (e) dementia with Lewy bodies, (f) Down's syndrome, (g) dementia associated with stroke, (h) dementia associated with Parkinson's disease, (i) dementia associated with beta-amyloid, or (j) preclinical Alzheimer's disease in a subject in need thereof.

### DETAILED DESCRIPTION OF THE INVENTION

The present invention is directed to compounds of Formula (I) as defined hereinbefore, and pharmaceutically acceptable addition salts and solvates thereof. The compounds of formula (I) are inhibitors of the beta-site APP-cleaving enzyme (also known as beta-site cleaving enzyme, BACE, BACE1, Asp2 or memapsin 2, or BACE2), and may be useful in the treatment of Alzheimer's disease, mild cognitive impairment, preclinical Alzheimer's disease, senility, dementia, dementia associated with stroke, dementia with Lewy bodies, Down's syndrome, dementia associated with Parkinson's disease, and dementia associated with beta-amyloid, preferably Alzheimer's disease, mild cognitive impairment or dementia, more preferably Alzheimer's disease.

In a particular embodiment, the invention is directed to compounds of Formula (I) as referred to herein, and the tautomers and the stereoisomeric forms thereof, wherein
R¹ is selected from the group consisting of hydrogen, —C₁₋₄alkyl, monohalo-C₁₋₄alkyl, and polyhalo-C₁₋₄alkyl;
R² is selected from the group consisting of hydrogen, —CN, —OC₁₋₄alkyl, —SO₂C₁₋₄alkyl, —SO₂cyclopropyl, and —SO(NCH₃)CH₃;
R³ is selected from the group consisting of hydrogen and C₁₋₄alkyl optionally substituted with 1-3 fluoro substituents;
R⁴ is hydrogen or fluoro;
Ar is homoaryl or heteroaryl;
wherein homoaryl is phenyl, or phenyl substituted with one, two or three substituents each independently selected from the group consisting of halo, cyano, C₁₋₄alkyl, C₁₋₄alkyloxy, monohalo-C₁₋₄alkyl, polyhalo-C₁₋₄alkyl, monohalo-C₁₋₄alkyloxy, and polyhalo-C₁₋₄alkyloxy;
heteroaryl is selected from the group consisting of pyridyl, pyrimidinyl, pyrazinyl, pyridazinyl, furanyl, thienyl, pyrrolyl, pyrazolyl, imidazolyl, triazolyl, tetrazolyl, thiazolyl, isothiazolyl, thiadiazolyl, oxazolyl, isoxazolyl, and oxadiazolyl, each optionally substituted with one, two or three substituents each independently selected from the group consisting of halo, cyano, C₁₋₄alkyl, C₂₋₄alkynyl, C₁₋₄alkyloxy, monohalo-C₁₋₄alkyl, polyhalo-C₁₋₄alkyl, monohalo-C₁₋₄alkyloxy,
polyhalo-C₁₋₄alkyloxy, and C₁₋₄alkyloxyC₁₋₄alkyloxy;
and the pharmaceutically acceptable acid addition salts thereof.

In another particular embodiment, the invention is directed to compounds of Formula (I) as referred to herein, and the tautomers and the stereoisomeric forms thereof, wherein
R¹ is selected from the group consisting of hydrogen and C₁₋₄alkyl;
R² is selected from the group consisting of hydrogen, cyano, and —SO₂C₁₋₄alkyl;
R³ is selected from the group consisting of hydrogen and C₁₋₄alkyl optionally substituted with 1-3 fluoro substituents;
R⁴ is hydrogen or fluoro;
Ar is selected from the group consisting of pyridyl, pyrimidinyl, pyrazinyl, and pyridazinyl, each optionally substituted with one, two or three substituents each independently selected from the group consisting of halo, cyano, C₁₋₄alkyl, C₁₋₄alkyloxy, monohalo-C₁₋₄alkyl, polyhalo-C₁₋₄alkyl, monohalo-C₁₋₄alkyloxy, and polyhalo-C₁₋₄alkyloxy;
and the pharmaceutically acceptable acid addition salts thereof.

In another particular embodiment, the invention is directed to compounds of Formula (I) as referred to herein, and the tautomers and the stereoisomeric forms thereof, wherein
R¹ is selected from the group consisting of hydrogen and C₁₋₄alkyl;
R² is selected from the group consisting of hydrogen, cyano, and —SO₂C₁₋₄alkyl;
R³ is selected from the group consisting of hydrogen and C₁₋₄alkyl optionally substituted with 1-3 fluoro substituents;
R⁴ is hydrogen or fluoro;
Ar is selected from the group consisting of pyridyl, pyrimidinyl, pyrazinyl, and pyridazinyl, each optionally substituted with one, two or three substituents each independently selected from the group consisting of halo, cyano, C₁₋₄alkyl, C₁₋₄alkyloxy, monohalo-C₁₋₄alkyl, polyhalo-C₁₋₄alkyl, monohalo-C₁₋₄alkyloxy, polyhalo-C₁₋₄alkyloxy, and triazolyl, in particular 1,2,4-triazol-1-yl;
and the pharmaceutically acceptable acid addition salts thereof.

In another particular embodiment, the invention is directed to compounds of Formula (I) as referred to herein, and the tautomers and the stereoisomeric forms thereof, wherein
R¹ is selected from the group consisting of hydrogen and C₁₋₄alkyl;
R² is selected from the group consisting of hydrogen, cyano, and —SO₂C₁₋₄alkyl;
R³ is selected from the group consisting of hydrogen and C₁₋₄alkyl optionally substituted with 1-3 fluoro substituents;
R⁴ is hydrogen or fluoro;
Ar is selected from the group consisting of pyridyl, pyrimidinyl, pyrazinyl, and pyridazinyl, each optionally substituted with one, two or three substituents each independently selected from the group consisting of halo, cyano, C₁₋₄alkyl, C₁₋₄alkyloxy, monohalo-C₁₋₄alkyl, polyhalo-C₁₋₄alkyl, monohalo-C₁₋₄alkyloxy, and polyhalo-C₁₋₄alkyloxy;
and the pharmaceutically acceptable acid addition salts thereof.

In a further embodiment, the invention is directed to compounds of Formula (I) as referred to herein, and the tautomers and the stereoisomeric forms thereof, wherein
R¹ is C₁₋₄alkyl;
R² is cyano or —SO₂C₁₋₄alkyl;
R³ is C₁₋₄alkyl optionally substituted with 1-3 fluoro substituents;
R⁴ is hydrogen or fluoro;
Ar is selected from the group consisting of pyridyl, pyrimidinyl, pyrazinyl, and pyridazinyl, each optionally substituted with one, two or three substituents each independently selected from the group consisting of halo, cyano, C₁₋₄alkyl, C₁₋₄alkyloxy, monohalo-C₁₋₄alkyl, polyhalo-C₁₋₄alkyl, monohalo-C₁₋₄alkyloxy, polyhalo-C₁₋₄alkyloxy; and triazolyl, in particular 1,2,4-triazol-1-yl;
and the pharmaceutically acceptable acid addition salts thereof.

In a further embodiment, the invention is directed to compounds of Formula (I) as referred to herein, and the tautomers and the stereoisomeric forms thereof, wherein
R¹ is C₁₋₄alkyl;
R² is cyano or —SO₂C₁₋₄alkyl;
R³ is C₁₋₄alkyl optionally substituted with 1-3 fluoro substituents;
R⁴ is hydrogen or fluoro;
Ar is selected from the group consisting of pyridyl, pyrimidinyl, pyrazinyl, and pyridazinyl, each optionally substituted with one, two or three substituents each independently selected from the group consisting of halo, cyano, C₁₋₄alkyl, C₁₋₄alkyloxy, monohalo-C₁₋₄alkyl, polyhalo-C₁₋₄alkyl, monohalo-C₁₋₄alkyloxy, and polyhalo-C₁₋₄alkyloxy;
and the pharmaceutically acceptable acid addition salts thereof.

In a further embodiment, the invention is directed to compounds of Formula (I) as referred to herein, and the tautomers and the stereoisomeric forms thereof, wherein
R¹ is C₁₋₄alkyl;
R² is cyano;
R³ is C₁₋₄alkyl optionally substituted with 1-3 fluoro substituents;
R⁴ is hydrogen or fluoro;
Ar is selected from the group consisting of pyridyl, pyrimidinyl, pyrazinyl, and pyridazinyl, each optionally substituted with one, two or three substituents each independently selected from the group consisting of halo, cyano, C₁₋₄alkyl, C₁₋₄alkyloxy, monohalo-C₁₋₄alkyl, polyhalo-C₁₋₄alkyl, monohalo-C₁₋₄alkyloxy, polyhalo-C₁₋₄alkyloxy; and triazolyl, in particular 1,2,4-triazol-1-yl;
and the pharmaceutically acceptable acid addition salts thereof.

In a further embodiment, the invention is directed to compounds of Formula (I) as referred to herein, and the tautomers and the stereoisomeric forms thereof, wherein
R¹ is C₁₋₄alkyl;
R² is cyano;
R³ is C₁₋₄alkyl optionally substituted with 1-3 fluoro substituents;
R⁴ is hydrogen or fluoro;
Ar is selected from the group consisting of pyridyl, pyrimidinyl, pyrazinyl, and pyridazinyl, each optionally substituted with one, two or three substituents each independently selected from the group consisting of halo, cyano, C₁₋₄alkyl, C₁₋₄alkyloxy, monohalo-C₁₋₄alkyl, polyhalo-C₁₋₄alkyl, monohalo-C₁₋₄alkyloxy, and polyhalo-C₁₋₄alkyloxy;
and the pharmaceutically acceptable acid addition salts thereof.

In another embodiment R¹ is hydrogen or methyl;
R² is hydrogen or cyano;
R³ is selected from the group consisting of methyl, fluoromethyl, difluoromethyl, trifluoromethyl, 1,1-difluoroethyl, 2-fluoro-2-propyl, and 1-fluorocyclopropyl.

In an embodiment, Ar is pyridyl or pyrazinyl, each optionally substituted with one, two or three substituents each independently selected from the group consisting of halo, cyano, C₁₋₄alkyl, C₁₋₄alkyloxy, monohalo-C₁₋₄alkyl, polyhalo-C₁₋₄alkyl, monohalo-C₁₋₄alkyloxy, and polyhalo-C₁₋₄alkyloxy; and all other variables are as described in Formula (I) herein.

In a further embodiment, Ar is pyridyl or pyrazinyl, each optionally substituted with one, two or three substituents each independently selected from the group consisting of cyano, monohalo-C₁₋₄alkyloxy, and polyhalo-C₁₋₄alkyloxy; and all other variables are as described in Formula (I) herein.

In a further embodiment, R¹ is —CH₃; R² is —CN; and R³ is —CH₃ or —CF₃; and all other variables are as described in Formula (I) herein.

In a further embodiment, the compounds of Formula (I) are in particular compounds of Formula (I-a), wherein R³ and the aryl moiety are projected above the plane of the drawing (with the bond shown with a bold wedge [symbol] ); or the compounds of Formula (I) are in particular compounds of Formula (I-b), wherein R³ and the aryl moiety are projected below the plane of the drawing (with the bond shown with a wedge of parallel lines [symbol] ); and all variables are as defined herein for compounds of Formula (I)

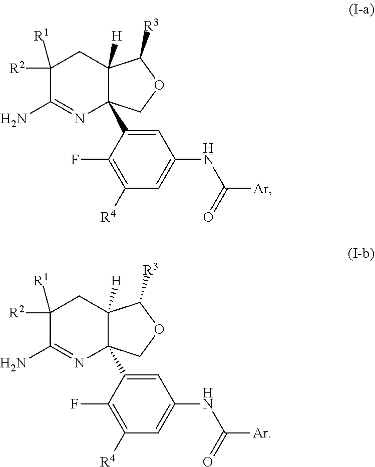

Preferred compounds of Formula (I) are those compounds according to the invention having Formula (I-a) as defined herein.

### Definitions

“Halo” shall denote fluoro, chloro and bromo; “C₁₋₄alkyl” shall denote a straight or branched saturated alkyl group having 1, 2, 3 or 4 carbon atoms, respectively e.g. methyl, ethyl, 1-propyl, 2-propyl, butyl, 1-methyl-propyl, 2-methyl-1-propyl, 1,1-dimethylethyl, and the like; “C₁₋₄alkyloxy” shall denote an ether radical wherein C₁₋₄alkyl is as defined before; “mono- and polyhaloC₁₋₄alkyl” shall denote C₁₋₄alkyl as defined before, substituted with 1 or with 1, 2, 3 or where possible with more halo atoms as defined before; “mono- and polyhaloC₁₋₄alkyloxy” shall denote an ether radical wherein mono- and polyhaloC₁₋₄alkyl are as defined before; “C₂₋₄alkynyl” shall denote an acyclic straight or branched hydrocarbon of 2, 3 or 4 carbon atoms and having a carbon-carbon triple bond.

The term “subject” as used herein, refers to an animal, preferably a mammal, most preferably a human, who is or has been the object of treatment, observation or experiment. As used herein, the term “subject” therefore encompasses patients, as well as asymptomatic or presymptomatic individuals at risk of developing a disease or condition as defined herein.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of Formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) or (I-a) is for instance specified as (R), this means that the compound is substantially free of the (S) isomer; when a compound of formula (I) or (I-a) is for instance specified as E, this means that the compound is substantially free of the Z isomer; when a compound of formula (I) or (I-a) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, trifluoromethylsulfonic acid, and undecylenic acid. Representative bases which may be used in the preparation of pharmaceutically acceptable addition salts include, but are not limited to, the following: ammonia, L-arginine, benethamine, benzathine, calcium hydroxide, choline, dimethylethanol-amine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The names of compounds were generated according to the nomenclature rules agreed upon by the Chemical Abstracts Service (CAS) or according to the nomenclature rules agreed upon by the International Union of Pure and Applied Chemistry (IUPAC).

The compounds according to formula (I) may be in dynamic equilibrium with their tautomeric form (I*) and form an inseparable mixture. Such tautomeric forms although not explicitly indicated in the above formula are intended to be included within the scope of the present invention.

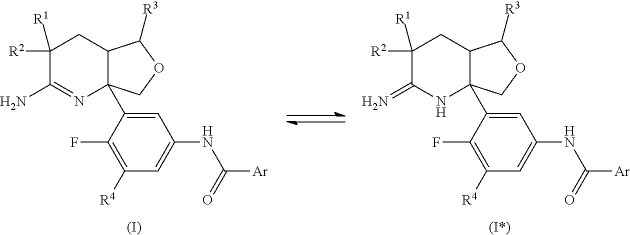

### Preparation of the Compounds

### Experimental Procedure 1

Final compounds according to Formula (I) can be prepared by reacting an intermediate of Formula (IIa) with a compound of Formula (IIIa) according to reaction scheme 1. The reaction is performed in a suitable reaction-inert solvent, such as, for example methanol (MeOH), in the presence of an acid, such as for example, HCl, and of a carboxyl activating agent such as for example, 1-ethyl-3-(3-dimethylaminopropyl) carbodiimide [EDCI, CAS 1892-57-7], under suitable conditions such as for example, stirring the reaction mixture at 25° C., until completion of the reaction, for example, 10 min.

Alternatively, the compounds of Formula (I) can be prepared by a Buchwald-Hartwig type coupling by reaction of an intermediate of Formula (IIb) with a compound of Formula (IIIb). The reaction is performed in a suitable reaction-inert solvent, such as for example, dioxane, in the presence of a suitable base, such as for example, potassium phosphate, a copper catalyst such as for example, copper(I) iodide, and a diamine such as for example, (1R,2R)-(−)-1,2-diaminocyclohexane or N,N′-dimethylethylenediamine, under thermal conditions such as for example, heating the reaction mixture at 100° C., until completion of the reaction, for example, for 16 h.

In reaction scheme 1 all variables are as defined in Formula (I) and X is a suitable leaving group, for example halo, in particular bromo.

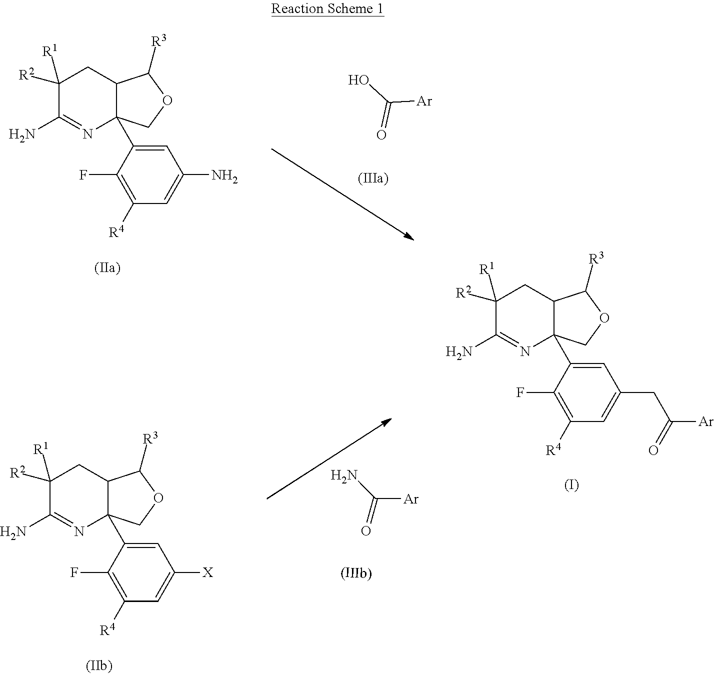

### Experimental Procedure 2

Intermediates of Formula (IIa) can be prepared by subjecting an intermediate of Formula (Va) to reducing conditions according to reaction scheme 2. Typical examples are reduction in the presence of a suitable catalyst, such as for example, palladium on carbon under hydrogen atmosphere, or the use of a reducing agent, such as for example, tin(II) chloride. The reactions are typically performed in a suitable solvent, such as for example MeOH, or in a solvent mixture, such as tetrahydrofuran (THF)/ethanol (EtOH). Thermal conditions such as for example, heating the reaction mixture, may improve the reaction outcome.

The intermediate of Formula (Va) can be prepared by nitration of an intermediate of Formula (IVa). A typical procedure involves the treatment of intermediate (IVa), dissolved in H₂SO₄, with a source of nitronium ions, such as for example, KNO₃, at low temperature, such as for example, 0° C.

The intermediate of Formula (IIa) can be alternatively prepared from intermediate of Formula (IIb), wherein X is a suitable halo, for example bromo, by a copper-catalyzed reaction with NaN₃. The reaction can be performed in a suitable reaction-inert solvent, such as for example, acetonitrile (MeCN), in the presence of a suitable base, such as for example, Na₂CO₃, a copper catalyst, such as for example copper(I) iodide and a diamine such as for example, N,N′-dimethylethylenediamine, under thermal conditions such as for example, heating the reaction mixture at 100° C., for example for 16 h.

In reaction scheme 2 all variables are as defined in Formula (I), and X is halo.

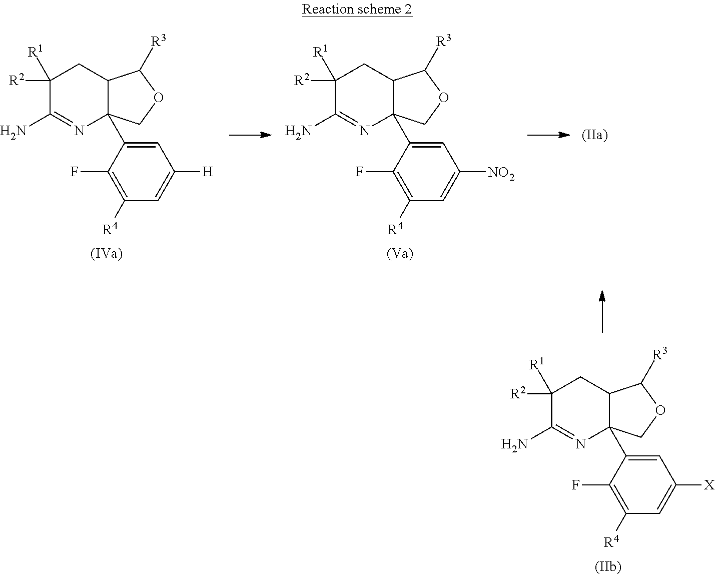

### Experimental Procedure 3

Intermediate compounds of Formulae (IIa) and (IIb) can be prepared according to a succession of steps, using a common intermediate of Formula (XII), depending on the different substituents present at R¹ and R².

Intermediate compounds of Formula (XII) can be prepared from starting materials that are commercially available or known in the art. For example, for the formation of intermediate (VI), when R³ is CH₃, suitable starting materials can be N,O-dimethylhydroxylamine.HCl and 2-[(1-methyl-2-propen-1-yl)oxy]-acetic acid, which can be reacted via a mixed anhydride with carbonyl diimidazole (CDI) under appropriate reaction conditions; when R³ is CF₃, suitable starting materials are 2-chloro-N-methoxy-N-methylacetamide and 3,3,3-trifluoro-1,2-epoxypropane, which can be typically reacted in a reaction-inert solvent such as THF, in the presence of a base such as lithium bis(trimethylsilyl)amide, typically at a temperature ranging from −30 to −20° C., then room temperature, until completion of the reaction.

The intermediate of Formula (VI) can be then reacted with a suitable halogenated benzene, in a reaction-inert solvent, such as THF, in the presence of a base, such as nBuLi, to form an intermediate of Formula (VII). The reaction is typically performed at a temperature of −78 to −60° C., followed by warming to room temperature, for a suitable time until completion of the reaction.

The intermediate of Formula (VII) can be then reacted with hydroxylamine.HCl under suitable reaction conditions, typically in MeOH in the presence of sodium acetate, to form intermediate of Formula (VII). This latter intermediate can be then reacted for example, with 1,4-dihydroxybenzene in xylenes under reflux, to form an intermediate of Formula (IX).

The intermediate of Formula (IX) can be subjected to treatment with zinc in the presence of acetic acid at a temperature typically around 0° C., to form an intermediate compound of formula (X). Protection of the amino group with a suitable protecting group (PG) and subsequent oxidation of the alcohol to form the aldehyde, for example, with Dess-Martin periodinane under art-known conditions, yields the intermediate of Formula (XII).

In reaction scheme 3 all variables are as defined in Formula (I), PG represents a suitable amino protecting group, and Z is hydrogen or halo, in particular bromo.

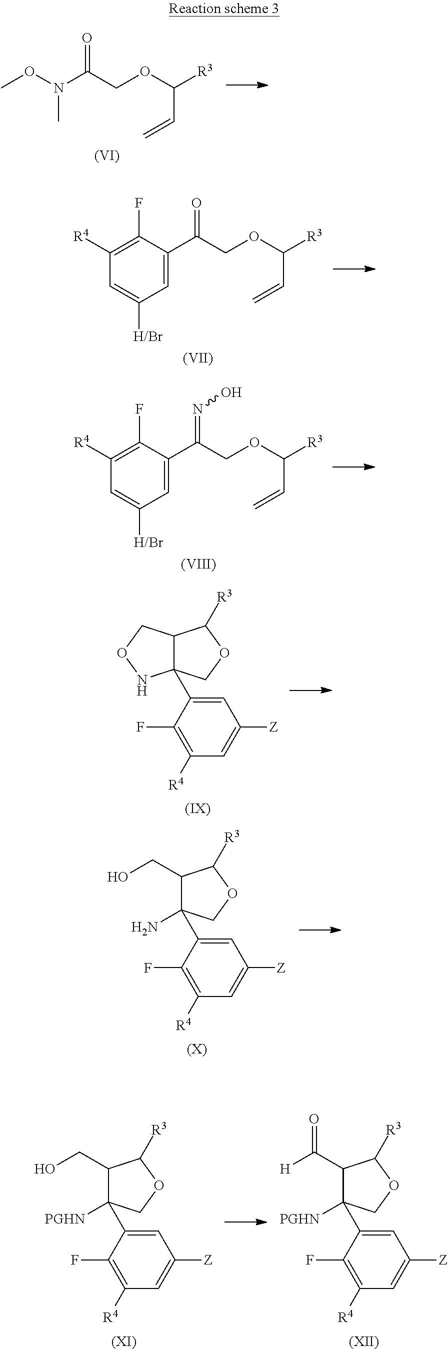

### Experimental Procedure 4

Intermediates of Formula (XII) can then be subjected to a sequence of steps to obtain intermediate compounds of Formula (IIa) and (IIb) which, depending on the definitions of R¹/R², are hereby referred to as intermediate compounds of Formulae (XV), (XIX), and (XXIII). Said intermediates (XV), (XIX), or (XXIII), can be subsequently reacted with a compound of Formula (IIIb) in order to yield the compounds of Formula (I), when Z=halo, as depicted in Reaction scheme 1; or can be subjected to the sequence of steps depicted in Reaction scheme 2, when Z=hydrogen, to obtain an intermediate of Formula (IIa), which can be further reacted with a compound of Formula (IIIa), according to Reaction scheme 1, in order to yield the compounds of Formula (I).
a) Formation of intermediate (II) wherein
   R¹=hydrogen, C₁₋₄alkyl, monohalo-C₁₋₄alkyl, or polyhalo-C₁₋₄alkyl and R²═—SO₂C₁₋₄alkyl, —SO₂cyclopropyl, and —SO(NCH₃)CH₃ (intermediate XV)

Intermediate compounds of Formula (XV) can be formed from intermediate (XII) in three steps. Thus, intermediate (XII) can be reacted with a suitable 2-(C₁₋₄alkylsulfonyl)acetonitrile or 2-(cyclopropylsulfonyl)-acetonitrile to yield an intermediate of Formula (XIII). The reaction can be performed for example in a reaction-inert solvent such as THF, in the presence of proline, typically under reflux, followed by reduction with for example, sodium borohydride, under reaction conditions known to the skilled person. Intermediate of Formula (XIII) can be optionally alkylated with an appropriate alkylating agent under reaction conditions known to the skilled person (R¹=C₁₋₄alkyl or fluorinated C₁₋₄alkyl), and subsequently, or directly (R¹=hydrogen), to deprotection under suitable conditions for cleavage of the amino protecting group, to yield an intermediate of Formula (XV).

In reaction scheme 4a all variables are as defined in Formula (I), PG represents a suitable amino protecting group, and Z is hydrogen or halo, in particular bromo.

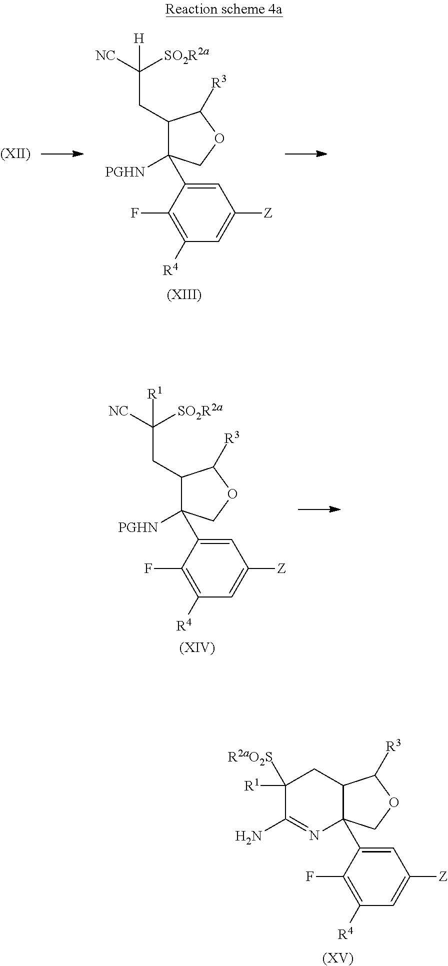

b) Formation of intermediates of formula (II) wherein R¹ and R² are hydrogen (intermediate XIX)

Intermediate compound of Formula (XIX) can be formed from intermediate (XII) in four steps. Thus, intermediate (XII) can be subjected to an olefination reaction (step A) with an appropriate phosphorous reagent, for example triethyl phosphonoacetate, in order to form an intermediate of Formula (XVI), wherein R typically represents methyl or ethyl. Cleavage of the amino protecting group (step B) under suitable reaction conditions can afford intermediate of Formula (XVII), which is then converted into the corresponding thioamide derivative of Formula (XVIII) following art-known thionation procedures (reaction step C); said conversion may conveniently be conducted by treatment of the said amides with a thionation agent such as, for example, phosphorous pentasulfide or 2,4-bis-(4-methoxyphenyl)-1,3-dithia-2,4-diphosphetane-2,4-disulfide [Lawesson's reagent, CAS 19172-47-5], in a reaction inert solvent such as, for example, tetrahydrofuran or 1,4-dioxane and the like, under thermal conditions such as, for example, heating the reaction mixture at 50° C., to completion of the reaction, for example, for 50 min. The amidine intermediates of formula (XI) may be conveniently prepared from the corresponding thioamide derivative of Formula (XVIII) following art-known thioamide-to-amidine conversion procedures (reaction step D). Said conversion may be conveniently conducted by treatment of the said thioamides with an ammonia source such as, for example, aqueous ammonia or ammonium chloride, in a suitable reaction-inert solvent such as, for example, water or MeOH and the like, under thermal conditions such as, for example, heating the reaction mixture at 60° C., for example for 6 h.

In reaction scheme 4b all variables are as defined in Formula (I), PG represents a suitable amino protecting group, R is an alkyl group, typically methyl or ethyl, and Z is hydrogen or halo, in particular bromo.

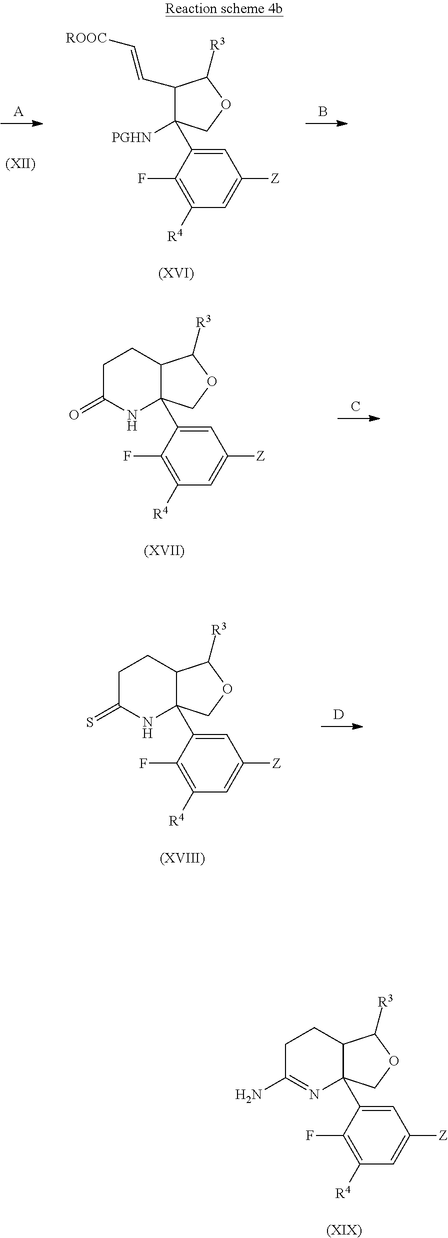

c) Formation of intermediates of Formula (II) wherein R¹=hydrogen, —C₁₋₄alkyl, monohalo-C₁₋₄alkyl, or polyhalo-C₁₋₄alkyl and R²═CN (intermediate XXIII)

Intermediate compound of Formula (XXIII) can be formed from intermediate (XII) in four steps. Thus, intermediate (XII) can be reacted with methyl cyanoacetate (step A) to form an intermediate of Formula (XX), under typical reaction conditions such as, for example, a reaction-inert solvent such as MeOH, in the presence of MgO at room temperature for a sufficient period of time to drive the reaction to completion. Intermediate (XX) can be reduced under art-known conditions (step B), for example, using sodium borohydride in a reaction inert solvent, such as THF, at an appropriate temperature, for example about −5° C. The resulting intermediate of Formula (XXI) can be optionally subjected to an alkylation reaction (Step C) under art-known conditions, for example by reaction with an appropriate alkyliodide reagent in the presence of a base, such as for example NaH in a reaction-inert solvent, such as THF, and subsequently—or directly—subjected to cleavage of the amino protecting group under suitable reaction conditions (step D), for example, in formic acid when the amino protecting group is tert-butyloxycarbonyl (Boc), to obtain the intermediate of Formula (XXIII). In reaction scheme 4c all variables are as defined in Formula (I), PG represents a suitable amino protecting group, and Z is hydrogen or halo, in particular bromo.

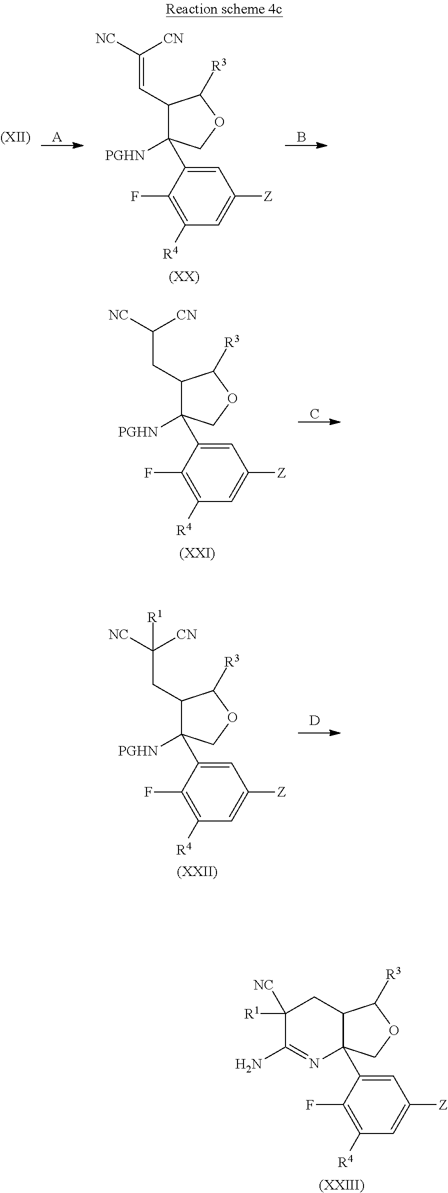

### Experimental Procedure 5

The intermediate of Formula (IX) can also be obtained by addition of the aryl moiety after performing the 1,3-dipolar cycloaddition as shown in reaction scheme 5. Alkylation of an intermediate alcohol of Formula (XXIV) with a haloacetaldehyde dialkyl acetal of Formula (XXV) yields the intermediate of Formula (XXVI). Treatment of the intermediate of Formula (XXVI) with an acid such as formic acid or acetic acid in an aqueous environment liberates an aldehyde, which can be condensed in situ with hydroxylamine HCl, typically in the presence of sodium acetate, to yield an intermediate of Formula (XXVII). This latter intermediate can then undergo a 1,3-dipolar cycloaddition to form an intermediate of Formula (XXVIII) by treatment with sodium hypochlorite in a suitable solvent such as dichloromethane, at an appropriate temperature, for example about 0° C. to room temperature. The intermediate of Formula (XXVIII) can be then reacted with a suitable halogenated benzene, in a reaction-inert solvent, such as THF, in the presence of a base, such as nBuLi, to form an intermediate of Formula (IX). The reaction is typically performed at a temperature of −78 to −60° C., for a suitable time until completion of the reaction.

In reaction scheme 5 all variables are as defined in Formula (I), Alk typically represents methyl or ethyl, X represents a reactive halogen such as chloro, bromo, or iodo, and Z is hydrogen.

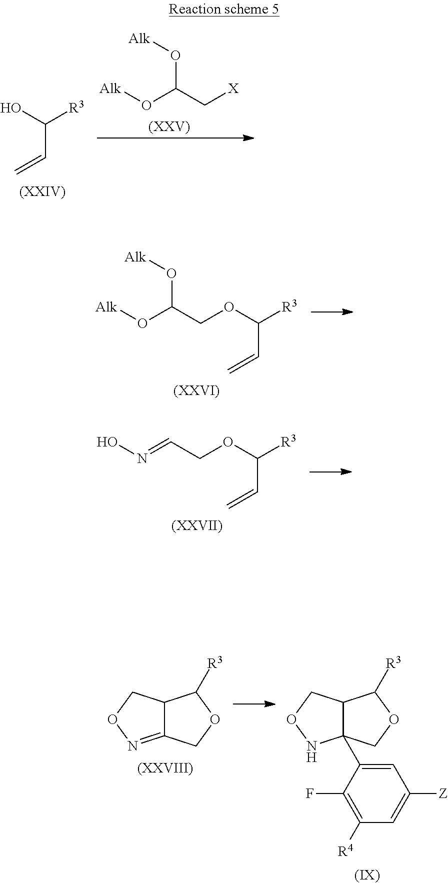

In an embodiment, R³ represents CH₂OPG, where PG is a protective group such as trityl or tert-butyldimethylsilyl, that can be deprotected easily to CH₂OH and converted at later stages in the synthesis route towards an R³ group as desired in the final compound.

### Pharmacology

The compounds of the present invention and the pharmaceutically acceptable compositions thereof inhibit BACE and therefore may be useful in the treatment or prevention of Alzheimer's Disease (AD), mild cognitive impairment (MCI), senility, dementia, dementia with Lewy bodies, cerebral amyloid angiopathy, multi-infarct dementia, Down's syndrome, dementia associated with Parkinson's disease, dementia of the Alzheimer's type, vascular dementia, dementia due to HIV disease, dementia due to head trauma, dementia due to Huntington's disease, dementia due to Pick's disease, dementia due to Creutzfeldt-Jakob disease, frontotemporal dementia, dementia pugilistica, and dementia associated with beta-amyloid.

As used herein, the term “treatment” is intended to refer to all processes, wherein there may be a slowing, interrupting, arresting or stopping of the progression of a disease or an alleviation of symptoms, but does not necessarily indicate a total elimination of all symptoms.

Preclinical Alzheimer's Disease:

In recent years the United States (US) National Institute for Aging and the International Working Group have proposed guidelines to better define the preclinical (asymptomatic) stages of AD (Dubois B, et al. Lancet Neurol. 2014; 13:614-629; Sperling, R A, et al. Alzheimers Dement. 2011; 7:280-292). Hypothetical models postulate that Aβ accumulation begins many years before the onset of overt clinical impairment. The key risk factors for elevated amyloid accumulation and development of AD are age (ie, 65 years or older), APOE genotype, and family history. Approximately one third of clinically normal older individuals over 75 years of age demonstrate evidence of Aβ accumulation on PET amyloid imaging studies or based upon CSF measurements. Similar findings are seen in large autopsy studies. These amyloid-positive (Aβ+) clinically normal individuals consistently demonstrate evidence of an “AD-like endophenotype” on other biomarkers, including elevations in CSF tau and phosphorylated tau (p-tau), disrupted functional network activity in both functional magnetic resonance imaging (MM) and resting state connectivity, fluorodeoxyglucose ¹⁸F (FDG) hypometabolism, cortical thinning, and accelerated rates of atrophy. Accumulating longitudinal data also strongly suggests that Aβ+ clinically normal individuals are at increased risk for cognitive decline and progression to mild cognitive impairment (MCI) and AD dementia. The Alzheimer's scientific community is of the consensus that these Aβ+ clinically normal individuals represent an early stage in the continuum of AD pathology. Thus, it has been argued that intervention with a therapeutic agent that decreases Aβ production is likely to be more effective if started at a disease stage before widespread neurodegeneration has occurred. A number of pharmaceutical companies are currently testing BACE inhibition in prodromal AD.

Thanks to evolving biomarker research, it is now possible to identify Alzheimer's disease at a preclinical stage before the occurrence of the first symptoms. All the different issues relating to preclinical Alzheimer's disease such as, definitions and lexicon, the limits, the natural history, the markers of progression and the ethical consequences of detecting the disease at the asymptomatic stage, are reviewed in Alzheimer's & Dementia 12 (2016) 292-323.

Two categories of individuals may be recognized in preclinical Alzheimer's disease. Cognitively normal individuals with amyloid beta evident on PET scans, or changes in CSF Abeta, tau and phospho-tau are defined as being in an “asymptomatic at risk state for Alzheimer's disease (AR-AD)”. Individuals with a fully penetrant dominant autosomal mutation for familial Alzheimer's disease are said to have “presymptomatic Alzheimer's disease”.

Thus, in an embodiment, the invention also relates to a compound according to the general Formula (I), in particular a compound of Formula (I-a), a stereoisomeric form thereof or a pharmaceutically acceptable acid or base addition salt thereof, for use in control or reduction of the risk of preclinical Alzheimer's disease, or prodromal Alzheimer's disease.

The invention also relates to a compound according to the general Formula (I), in particular a compound of Formula (I-a), a stereoisomeric form thereof or a pharmaceutically acceptable acid or base addition salt thereof, for use in the treatment or prevention of diseases or conditions selected from the group consisting of AD, MCI, preclinical Alzheimer's disease, senility, dementia, dementia with Lewy bodies, cerebral amyloid angiopathy, multi-infarct dementia, Down's syndrome, dementia associated with Parkinson's disease, dementia of the Alzheimer's type, and dementia associated with beta-amyloid.

The invention also relates to a compound according to the general Formula (I), in particular a compound of Formula (I-a), a stereoisomeric form thereof or a pharmaceutically acceptable acid or base addition salt thereof, for use in the treatment, prevention, amelioration, control or reduction of the risk of diseases or conditions selected from the group consisting of AD, MCI, preclinical Alzheimer's disease, senility, dementia, dementia with Lewy bodies, cerebral amyloid angiopathy, multi-infarct dementia, Down's syndrome, dementia associated with Parkinson's disease, dementia of the Alzheimer's type, and dementia associated with beta-amyloid.

As already mentioned hereinabove, the term “treatment” does not necessarily indicate a total elimination of all symptoms, but may also refer to symptomatic treatment in any of the disorders mentioned above. In view of the utility of the compound of Formula (I), in particular the compound of Formula (I-a), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a therapeutically effective amount of a compound of Formula (I), in particular of a compound of Formula (I-a), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a therapeutically effective amount of a compound according to the invention to a subject in need thereof.

The invention also relates to a method for modulating beta-site amyloid cleaving enzyme activity, comprising administering to a subject in need thereof, a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent Alzheimer's disease or the symptoms thereof, may be administered alone or in combination with one or more additional therapeutic agents. Combination therapy includes administration of a single pharmaceutical dosage formulation which contains a compound of Formula (I), in particular a compound of Formula (I-a), and one or more additional therapeutic agents, as well as administration of the compound of Formula (I), in particular of the compound of Formula (I-a), and each additional therapeutic agent in its own separate pharmaceutical dosage formulation. For example, a compound of Formula (I), in particular a compound of Formula (I-a), and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5™ ) of the American Psychiatric Association utilizes terms such as neurocognitive disorders (NCDs) (both major and mild), in particular, neurocognitive disorders due to Alzheimer's disease, due to traumatic brain injury (TBI), due to Lewy body disease, due to Parkinson's disease or to vascular NCD (such as vascular NCD present with multiple infarctions). Such terms may be used as an alternative nomenclature for some of the diseases or conditions referred to herein by the skilled person.

### Pharmaceutical Compositions

The present invention also provides compositions for preventing or treating diseases in which inhibition of beta-site APP-cleaving enzyme is beneficial, such as Alzheimer's disease (AD), mild cognitive impairment, preclinical Alzheimer's disease, senility, dementia, dementia with Lewy bodies, Down's syndrome, dementia associated with stroke, dementia associated with Parkinson's disease and dementia associated with beta-amyloid. Said compositions comprising a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of Formula (I), in particular on the particular compound of Formula (I-a), used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to Formula (I), more in particular according to Formula (I-a), used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I), in particular of Formula (I-a), that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### Experimental Part

Hereinafter, the term “m.p.” means melting point, “min” means minutes, “AcOH” means acetic acid, “aq.” means aqueous, “DIBAL” means diisobutylaluminium hydride, “r.m.” means reaction mixture, “r.t.” or “RT” means room temperature, “rac” or “RS” means racemic, “sat.” means saturated, “SFC” means supercritical fluid chromatography, “SFC-MS” means supercritical fluid chromatography/mass spectrometry, “LC-MS” means liquid chromatography/mass spectrometry, “HPLC” means high-performance liquid chromatography, “NP” means normal phase, “RP” means reversed phase, “Rₜ” means retention time (in minutes), “[M+H]⁺” means the protonated mass of the free base of the compound, “wt” means weight, “THF” means tetrahydrofuran, “EtOAc” means ethyl acetate, “DCE” means dichloroethane, “DCM” means dichloromethane, “MeOH” means methanol, “MW” means microwave, “org.” means organic, “sol.” means solution, “Boc” means tert-butoxycarbonyl, “TLC” means thin layer chromatography, “Pd/C” means palladium on carbon, “EtOH” means ethanol, “DIPE” means diisopropyl ether, “EDCI.HCl” means 1-ethyl-3-(3-dimethylaminopropyl)carbodiimide hydrochloride, “DMTMM” means 4-(4,6-Dimethoxy-1,3,5-triazin-2-yl)-4-methylmorpholinium chloride, “TFA” means trifluoroacetic acid, “prep” means preparative, “NMP” means N-methylpyrrolidone, “DIPEA” means diisopropylethylamine, “DMAP” means 4-dimethylaminopyridine, “CDI” means 1,1′-carbonyldiimidazole, “TEMPO” means 2,2,6,6-tetramethylpiperidine-N-oxide, “NaOAc” means sodium acetate, “Tr” means trityl/triphenylmethyl, and “Xtalfluor-M®” means difluoro(morpholino)sulfonium tetrafluoroborate.

Whenever the notation “RS” is indicated herein, it denotes that the compound is a racemic mixture at the indicated centre, unless otherwise indicated. The stereochemical configuration for centres in some compounds has been designated “R” or “S” when the mixture(s) was separated; for some compounds, the stereochemical configuration at indicated centres has been designated as “*R” or “*S” when the absolute stereochemistry is undetermined although the compound itself has been isolated as a single stereoisomer and is enantiomerically/diastereomerically pure. The enantiomeric excess of compounds reported herein was determined by analysis of the racemic mixture by supercritical fluid chromatography (SFC) followed by SFC comparison of the separated enantiomer(s).

In intermediates/compounds wherein bonds are indicated either with a bold wedge or a wedge of parallel lines while the stereocentres are designated RS, the representation indicates that the sample is a mixture of stereoisomers, one stereoisomer having the indicated substituents or groups projected above or below the plane of the drawing as represented, one stereoisomer having the substituents or groups in the opposite projection below or above the plane of the drawing, e.g.

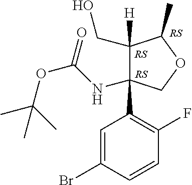 [SMILES: C[C@H]1OC[C@@](NC(=O)OC(C)(C)C)(c2cc(Br)ccc2F)[C@@H]1CO]

represents a mixture of

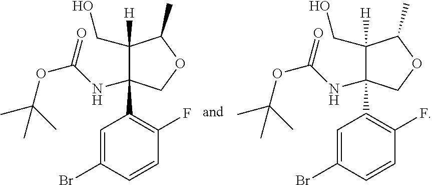 [SMILES: C[C@H]1OC[C@@](NC(=O)OC(C)(C)C)(c2cc(Br)ccc2F)[C@@H]1CO ; C[C@@H]1OC[C@](NC(=O)OC(C)(C)C)(c2cc(Br)ccc2F)[C@H]1CO]

The absolute configuration of chiral centres (indicated as R and/or 5) can be rationalized. The synthesis of all final compounds started from intermediates of known absolute configuration in agreement with literature precedent or obtained from appropriate synthetic procedures. The assignment of the absolute configuration of additional stereocentres could then be assigned by standard NMR methods.

A. Preparation of the Intermediates

Preparation of Intermediate 1 (I-1)

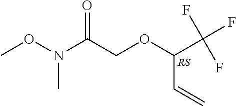

To a solution of trimethylsulfonium iodide (41.51 g, 203.9 mmol) in THF (335.3 mL) at −30° C. was added lithium bis(trimethylsilyl)amide (1M in heptane, 203.9 mL, 203.9 mmol) portionwise over 45 mins. After stirring for 30 mins, 3,3,3-trifluoro-1,2-epoxypropane (14 g, 124.9 mmol) was added at −20° C. over 15 min, and the mixture was allowed to warm to RT and stirred for 3 h and 10 min. The slurry was then added portionwise to an ice-cold solution of 2-chloro-N-methoxy-N-methylacetamide (28.05 g, 203.9 mmol) in NMP (75.7 mL). The resulting mixture was allowed to warm to RT and stirred for 1 day before dilution with EtOAc. The organic layer was washed with NaHCO₃ (sat. aq. sol.) and the washings extracted with EtOAc. The organic layers were combined, dried (MgSO₄) and evaporated. The residue was purified by column chromatography (silica gel; n-heptane/EtOAc 100/0 to 70/30) to yield I-1 (13.8 g, 49%).

Preparation of Intermediate 2 (I-2)

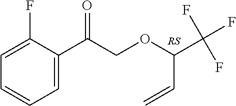 [SMILES: C=CC(OCC(=O)c1ccccc1F)C(F)(F)F]

A solution of nBuLi (2.5 M in hexanes, 21.8 mL, 58.9 mmol) was added dropwise over 25 min to a solution containing 1-bromo-2-fluorobenzene (10.31 g, 58.9 mmol) in THF (151.5 mL) under a N₂ atmosphere at −78° C. The reaction mixture was allowed to warm to −60° C. and stirred for 60 min. I-1 (10.3 g, 45.3 mmol) in THF (25.0 mL) was added dropwise to the reaction solution, and after stirring at −60° C. for 2 h, aqueous NH₄Cl was added, followed by warming to RT. Brine was added, and the mixture was extracted with EtOAc. The combined organic portions were dried (MgSO₄), evaporated, and the residue was purified by column chromatography (silica gel; hexanes/EtOAc 99/1 to 90/10) to obtain I-2 (9.5 g, 40%).

Preparation of Intermediate 3 (I-3)

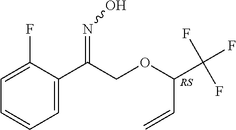 [SMILES: C=CC(OCC(=NO)c1ccccc1F)C(F)(F)F]

I-2 (4.5 g, 17.2 mmol) was dissolved in MeOH (69.5 mL). Then hydroxylamine hydrochloride (2.03 g, 29.2 mmol) and NaOAc (2.82 g, 34.3 mmol) were added. The reaction mixture was heated to 50° C. for 90 min, then cooled to RT, concentrated under vacuum and the residue was dissolved in DCM, concentrated and purified by column chromatography (silica gel; heptane/EtOAc 90/2 to 85/15) to yield I-3 (4.64 g, 98%) as a mixture of geometric isomers.

Preparation of Intermediate 4 (I-4)

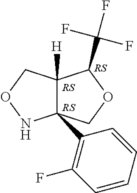 [SMILES: Fc1ccccc1[C@]12CO[C@H](C(F)(F)F)[C@H]1CON2]

I-3 (5.69 g, 20.2 mmol) was dissolved in xylenes (480 mL), then 1,4-dihydroxybenzene (0.633 g, 5.8 mmol) was added. The reaction mixture was refluxed for 21 h at 140° C. The solvent was cooled to RT and evaporated under reduced pressure. The residue was purified by flash column chromatography (silica gel, NP, Biotage flash purification system; n-heptane/EtOAc 100/0 to 70/30). The product fractions were collected and the solvent was evaporated to yield I-4 (4.94 g, 87%).

Preparation of Intermediate 5 (I-5)

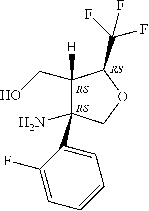 [SMILES: N[C@@]1(c2ccccc2F)CO[C@H](C(F)(F)F)[C@H]1CO]

To I-4 (10 g, 36.1 mmol) was added acetic acid (385.4 mL), and the mixture was cooled to 0° C. on an ice-bath. Zn (16.51 g, 252.51 mmol) was then added, and the reaction mixture was stirred and allowed to reach RT, then it was further stirred for 2 h at RT. EtOAc was added, the reaction was filtered over dicalite® and concentrated under reduced pressure. The residue was dissolved in DCM and basified by careful addition of aq. NH₃, the org layer was separated, dried (MgSO₄), filtered and the solvent evaporated under reduced pressure. The residue was purified by flash column chromatography (silica gel, NP, Biotage flash purification system; n-heptane/EtOAc 100/0 to 0/100). The product fractions were collected and the solvent was evaporated to yield I-5 (6.95 g, 69%).

Preparation of Intermediate 6 (I-6)

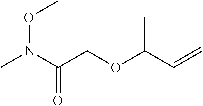

Two batches: To a solution of 2-[(1-methyl-2-propen-1-yl)oxy]-acetic acid ([77927-91-4], 533.57 g, 4.10 mol) in DCM (4.00 L) was added CDI (811.07 g, 5.00 mol) at 0° C. and the mixture was stirred at this temperature for 30 min. Then N,O-dimethylhydroxylamine.HCl (531.89 g, 5.45 mol) was added into the mixture, the final mixture was stirred at 20° C. for 1.5 h. HCl (1N, 2 L) was added, and the organic layer was extracted with DCM (2×2 L), dried (Na₂SO₄), and concentrated to give a crude that was purified on silica gel (petroleum ether/EtOAc 10/1 to 3/1) to yield I-6 (570.00 g total from two batches, 40%) as yellow oil.

Preparation of Intermediate 7 (I-7)

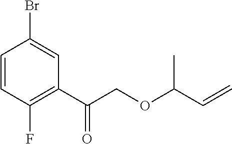 [SMILES: C=CC(C)OCC(=O)c1cc(Br)ccc1F]

I-7 was prepared according to a procedure analogous to that described for I-2, starting from I-6 and 4-bromo-1-fluoro-2-iodo-benzene.

Preparation of Intermediate 8 (I-8)

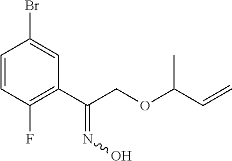 [SMILES: C=CC(C)OCC(=NO)c1cc(Br)ccc1F]

I-8 was prepared according to a procedure analogous to that described for I-3, starting from I-7.

Preparation of Intermediate 9 (I-9)

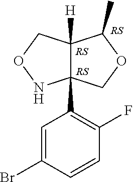 [SMILES: C[C@H]1OC[C@]2(c3cc(Br)ccc3F)NOC[C@H]12]

I-9 was prepared according to a procedure analogous to that described for I-4, starting from I-8.

Preparation of Intermediate 10 (I-10)

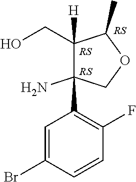 [SMILES: C[C@H]1OC[C@@](N)(c2cc(Br)ccc2F)[C@@H]1CO]

I-10 was prepared according to a procedure analogous to that described for I-5, starting from I-9.

Preparation of Intermediate 11 (I-11)

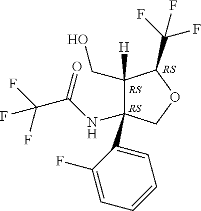 [SMILES: O=C(N[C@@]1(c2ccccc2F)CO[C@H](C(F)(F)F)[C@H]1CO)C(F)(F)F]

To a solution of I-5 (6 g, 21.5 mmol) in DCM (72.0 mL) at 0° C. was added DIPEA (7.41 mL, 43.0 mmol), followed by dropwise addition of trifluoroacetic anhydride (4.5 mL, 32.2 mmol). The resulting mixture was stirred at RT for 2 h. Water was added and the org layer was separated, washed with 1 N HCl and with a mixture of brine and sat. aq. NaHCO₃. Then the org layer was dried (MgSO₄), filtered and the solvent evaporated. The residue was purified by column chromatography (silica gel; n-heptane/EtOAc 100/0 to 40/60). The product fractions were collected and the solvent was evaporated under reduced pressure to yield I-11 (7g, 87%).

Preparation of Intermediate 12 (I-12)

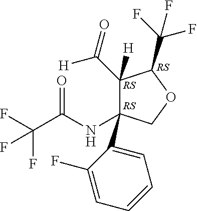 [SMILES: O=C[C@@H]1[C@@H](C(F)(F)F)OC[C@@]1(NC(=O)C(F)(F)F)c1ccccc1F]

Dess-Martin periodinane (3.80 g, 9.0 mmol) was added portion-wise to a solution of I-11 (2.8 g, 7.5 mmol) in DCM (52.4 mL) at 0° C. The mixture was stirred at 0° C. for 10 min and at RT for 2 h. The reaction was quenched with a 10% solution of Na₂S₂O₃. The org layer was separated and washed with saturated NaHCO₃ solution, then DCM was added and the org layer was washed again with sat. NaHCO₃ solution. The org layer was dried (MgSO₄), filtered off and concentrated. The crude was purified by flash column chromatography with solid loading (silica; EtOAc/heptane 0/100 to 50/50). The desired fractions were collected and concentrated in vacuo to yield I-12 (2.7 g, 97%).

Preparation of Intermediate 13 (I-13)

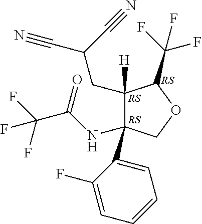

To a stirred solution of I-12 (2.57 g, 6.9 mmol) in MeOH (51.4 mL) at 0° C., was added MgO (416.3 mg, 10.3 mmol) and Ti(iPrO)₄ (6.1 mL, 20.7 mmol), then malononitrile (909.8 mg, 13.8 mmol), followed by addition of NaBH₃CN (562.5 mg, 9.0 mmol), and the reaction mixture was stirred at 0° C. for 2 h. Water, DCM and dicalite® were added, the reaction mixture was filtered, rinsed with DCM, and to the filtrate some extra water and DCM were added. The org layer was separated, dried (MgSO₄), filtered off and the filtrate was concentrated under reduced pressure to give a residue that was purified by column chromatography (NP, 80 g silica; heptane/EtOAc 100/0 to 50/50). The product fractions were collected and the solvent was evaporated under reduced pressure to yield I-13 (2.05 g, 70%).

Preparation of Intermediate 14 (I-14)

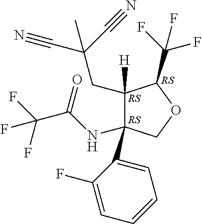 [SMILES: CC(C#N)(C#N)C[C@@H]1[C@@H](C(F)(F)F)OC[C@@]1(NC(=O)C(F)(F)F)c1ccccc1F]

To a stirred solution of I-13 (1.70 g, 4.0 mmol) in dry THF (152.3 mL) under N₂ atmosphere at 0° C. was added NaH (60% dispersion in mineral oil, 224.88 mg, 5.6 mmol), and the mixture was stirred for 15 min at 0° C. under N₂ atmosphere. CH₃I (350.0 μL, 5.6 mmol) was then added, and the mixture was stirred for 1 h at 0° C. Water and EtOAc were then added, and the org layer was separated, dried (MgSO₄), filtered and the solvent was evaporated under reduced pressure to yield a mixture (1.70 g, 97%) containing I-14 (73% purity).

Preparation of Intermediate 15 (I-15)

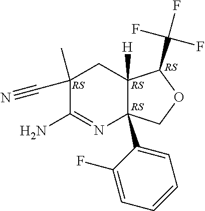 [SMILES: CC1(C#N)C[C@@H]2[C@@H](C(F)(F)F)OC[C@]2(c2ccccc2F)N=C1N]

To a stirred solution of I-14 (1.70 g, 3.9 mmol) in THF (68 mL) were added K₂CO₃ (2.69 g, 19.4 mmol) and distilled water (13.6 mL), and the reaction mixture was stirred at 60° C. for 90 min. The reaction mixture was then allowed to reach RT, and DCM and H₂O were added. The org layer was separated, washed with brine, the combined aq. layers were extracted with DCM and the combined org. layers were dried (MgSO₄), filtered and the solvent was evaporated under reduced pressure to yield a residue that was purified by column chromatography (silica gel; DCM/NH₃ in MeOH (7N) in DCM 100/0 to 90/10). The product fractions were collected and the solvent was evaporated under reduced pressure to yield I-15 (1 g, 75%).

Preparation of Intermediate 16 (I-16)

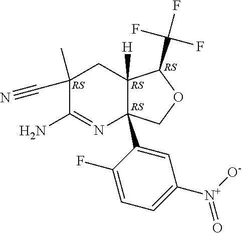 [SMILES: CC1(C#N)C[C@@H]2[C@@H](C(F)(F)F)OC[C@]2(c2cc([N+](=O)[O-])ccc2F)N=C1N]

To a stirred solution of I-15 (1 g, 2.9 mmol) in H₂SO₄ (20 mL) at 0° C. was added KNO₃ (325.8 mg, 3.2 mmol) and the reaction mixture was stirred at 0° C. for 15 min. DCM was added and the reaction mixture was basified at 0° C. by addition of Na₂CO₃ in solution and solid (caution!). The org layer was separated, dried (MgSO₄), filtered and the solvent was evaporated under reduced pressure to yield I-16 (1 g, 88%), which was used without further purification.

Preparation of Intermediate 17 (I-17)

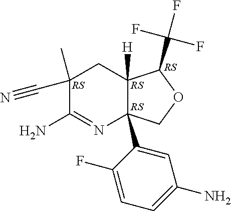 [SMILES: CC1(C#N)C[C@@H]2[C@@H](C(F)(F)F)OC[C@]2(c2cc(N)ccc2F)N=C1N]

Procedure 1: To a stirred solution of I-16 (600 mg, 1.6 mmol) in EtOAc (187.7 mL), under a N₂ atmosphere was added Pd/C (10%, 198.5 mg, 0.19 mmol). The reaction mixture was stirred for 6.5 h at RT under a H₂ atmosphere. The catalyst was filtered off over dicalite under N₂ atmosphere, and the org layer was concentrated in vacuo. The residue was purified by column chromatography (silica gel; DCM/7N NH₃ in MeOH in DCM 100/0 to 90/10). The product fractions were collected and the solvent was evaporated under reduced pressure to yield I-17 (510 mg, 92%).

Procedure 2: To a stirred solution of I-16 (1 g, 2.6 mmol) in MeOH (12.7 mL) were added H₂O (4 mL), Iron (1.157 g, 20.7 mmol) and NH₄Cl (1.504 g, 28.1 mmol), and the reaction mixture was stirred at 70° C. for 1 h. The reaction mixture was then allowed to cool down to RT and MeOH and DCM were added, it was then filtered over dicalite and the org layer was washed with water, dried (MgSO₄), filtered and the solvent was evaporated to yield I-17 (750 mg, 81%) which was used without further purification.

Preparation of Intermediate 18 (I-18)

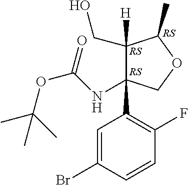 [SMILES: C[C@H]1OC[C@@](NC(=O)OC(C)(C)C)(c2cc(Br)ccc2F)[C@@H]1CO]

To a stirred solution of I-10 (20 g, 65.8 mmol) in THF (296.24 mL) were added Et₃N (13.71 mL, 98.6 mmol) and Boc₂O (18.662 g, 85.5 mmol) and the reaction mixture was stirred for 40 h. Additional Boc₂O (7.17 g, 32.9 mmol) was then added and the reaction mixture was stirred for a further 2 h. Sat. aq. sol. NaHCO₃ was added, the org layer was separated, dried (MgSO₄), filtered and the solvent was removed in vacuo. The residue was purified by flash column chromatography (n-heptane/EtOAc 100/0 to 50/50). The product fractions were collected and the solvent was removed in vacuo to yield I-18 (18.6 g, 70%).

Preparation of Intermediate 19 (I-19)

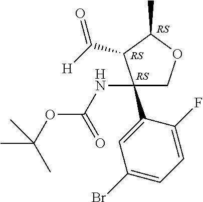 [SMILES: C[C@H]1OC[C@@](NC(=O)OC(C)(C)C)(c2cc(Br)ccc2F)[C@@H]1C=O]

Dess-Martin periodinane (12.84 g, 30.276 mmol) was added portion-wise over 5 min to a solution of I-18 (10.2 g, 25.2 mmol) in DCM (171.03 mL) at 0° C. The mixture was stirred at 0° C. for 10 min and at RT for 2 h. The mixture was treated with sat. sol. Na₂S₂O₃ (75 mL) and sat. sol. NaHCO₃ (75 mL), stirred for 15 min and extracted with DCM. The org layer was separated, dried (MgSO₄), filtered and concentrated in vacuo. The residue was purified by flash column chromatography (heptane/EtOAc 100/0 to 70/30). The desired fractions were collected and concentrated in vacuo to yield I-19 (7.7 g, 76%) as a white powder.

Preparation of Intermediate 20 (I-20)

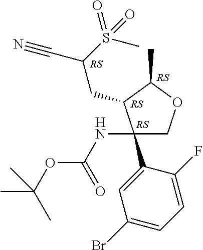 [SMILES: C[C@H]1OC[C@@](NC(=O)OC(C)(C)C)(c2cc(Br)ccc2F)[C@@H]1CC(C#N)S(C)(=O)=O]

A mixture of I-19 (4.1 g, 10.2 mmol), 2-(methylsulfonyl)acetonitrile (1.214 g, 10.2 mmol) and DL-proline (984 mg, 8.6 mmol) in THF (82 mL) was heated and stirred for 6 h under reflux. The reaction mixture was cooled to 0° C., and NaBH₄ (578.4 mg, 15.3 mmol) was added thereto. The mixture was stirred at RT for 6 h. After the reaction mixture was cooled to 0° C., water (10 mL) and EtOAc (30 mL) were added. The reaction mixture was filtered over dicalite®. The solvent was evaporated under reduced pressure to afford a mixture of two diastereomers and some unreacted starting material. This residue was purified by column chromatography (silica gel; n-heptane/EtOAc 100/0 to 70/30). The product fractions were collected and the solvent was evaporated under reduced pressure to afford I-20 ((0.974 g, 19%; and a 2/3 diastereomeric mixture (2.334 g, 45%)).

Preparation of Intermediate 21 (I-21)

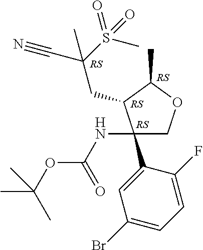 [SMILES: C[C@H]1OC[C@@](NC(=O)OC(C)(C)C)(c2cc(Br)ccc2F)[C@@H]1CC(C)(C#N)S(C)(=O)=O]

To a stirred solution of I-20 (505.4 mg, 1 mmol) in THF (15 mL) under N₂ atmosphere at 0° C. was added NaH (60% dispersion in mineral oil, 48 mg, 1.2 mmol), and the mixture was stirred for 15 min at 0° C. under Na atmosphere. CH₃I (75 μL, 1.2 mmol) was then added, and the mixture was stirred for 2 h at 0° C. H₂O and EtOAc were added. The organic layer was separated, dried (MgSO₄), filtered off and the solvent was evaporated under reduced pressure to afford a crude that was purified by column chromatography (silica gel; heptanes/EtOAc 10/0 to 7/3) to afford I-21 (300 mg, 58%).

Preparation of Intermediate 22 (I-22)

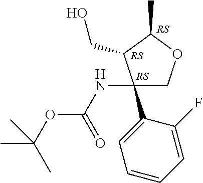 [SMILES: C[C@H]1OC[C@@](NC(=O)OC(C)(C)C)(c2ccccc2F)[C@@H]1CO]

To a stirred suspension of Pd/C (10%, 4.108 g, 3.9 mmol) in MeOH (25 mL) under N₂ atmosphere was added a solution of I-18 (15.605 g, 0.04 mmol) in MeOH (25 mL) was added, then Et₃N (21.46 mL, 154.4 mmol) was added. The reaction mixture was stirred at RT under H₂ atmosphere for 30 min (until 1 eq. was taken up). The catalyst was filtered off over dicalite® under N₂ atmosphere. The solvent was removed in vacuo, the residue was dissolved in DCM and sat. aq. sol. NaHCO₃ was added. The org layer was separated and the aq. layer was extracted further with DCM. The org layers were combined, dried (MgSO₄), filtered and the solvent was removed in vacuo. The reaction mixture was purified by flash column chromatography (n-heptane/EtOAc 100/0 to 50/50. The product fractions were collected and concentrated in vacuo to yield I-22 (11.76 g, 94%).

Preparation of Intermediate 23 (I-23)

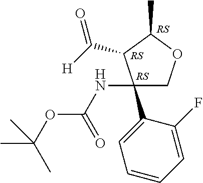 [SMILES: C[C@H]1OC[C@@](NC(=O)OC(C)(C)C)(c2ccccc2F)[C@@H]1C=O]

Dess-Martin periodinane (33.15 g, 78.2 mmol) was added portion-wise over 5 min to a stirred solution of I-22 (11.22 g, 34.5 mmol) in DCM (233.75 mL) at 0° C. The mixture was stirred at 0° C. for 10 min and at RT for 5 h. The mixture was treated with sat. sol Na₂S₂O₃ solution (100 mL) and sat. sol. NaHCO₃ (100 mL), stirred for 15 min and extracted with DCM. The org layer was separated, dried (MgSO₄), filtered and concentrated in vacuo. The residue was purified by flash column chromatography (heptane/EtOAc 100/0 to 60/40). The desired fractions were collected and concentrated in vacuo to yield I-23 (11 g, 99%) as a colorless gel.

Preparation of Intermediate 24 (I-24)

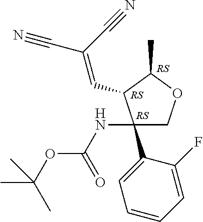 [SMILES: C[C@H]1OC[C@@](NC(=O)OC(C)(C)C)(c2ccccc2F)[C@@H]1C=C(C#N)C#N]

To a stirred solution of I-23 (4 g, 12.4 mmol) in MeOH (75 mL) were added MgO (301.9 mg, 7.5 mmol) and methyl cyanoacetate (817.2 mg, 12.4 mmol), and the reaction mixture was stirred for 1 h at RT. The reaction mixture was then filtered over dicalite® and the solvent was evaporated under reduced pressure to yield I-24 (4.4 g, 96%), which was used without further purification.

Preparation of Intermediate 25 (I-25)

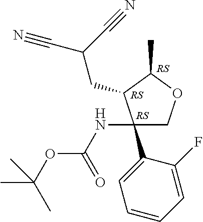 [SMILES: C[C@H]1OC[C@@](NC(=O)OC(C)(C)C)(c2ccccc2F)[C@@H]1CC(C#N)C#N]

To a stirred solution of I-24 (4.4 g, 11.9 mmol) in THF (220 mL) at −5° C., was added NaBH₄ (672.3 mg, 17.8 mmol), and the reaction mixture was stirred for 30 min at 0° C. The reaction mixture was then diluted with EtOAc and acidified by addition of HCl (2M) and H₂O. The org layer was separated, dried (MgSO₄), filtered and the solvent was evaporated under reduced pressure. The reaction mixture was purified with column chromatography (silica gel; n-heptane/EtOAc 100/0 to 65/35). The product fractions were collected and the solvent was evaporated under reduced pressure to yield I-25 (3.5 g, 79%).

Preparation of Intermediate 26 (I-26)

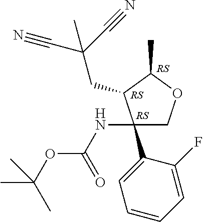 [SMILES: C[C@H]1OC[C@@](NC(=O)OC(C)(C)C)(c2ccccc2F)[C@@H]1CC(C)(C#N)C#N]

To a stirred solution of I-25 (3.5 g, 9.4 mmol) in dry THF (105 mL) under N₂ atmosphere at 0° C. was added NaH (60% dispersion in mineral oil, 487.3 mg, 12.2 mmol), and the mixture was stirred for 15 min at 0° C. under N₂ atmosphere. CH₃I (875.2 μL, 14.1 mmol) was added, the mixture was further stirred for 2 h at 0° C., then H₂O followed by EtOAc were added. The org layer was separated, dried (MgSO₄), filtered off and the solvent was evaporated under reduced pressure. The reaction mixture was purified with column chromatography (silica gel; n-heptane/EtOAc 100/0 to 65/35). The product fractions were collected and the solvent was evaporated under reduced pressure to yield I-26 (2.8 g, 77%).

Preparation of Intermediates 27a (I-27a) and 27b (I-27b)

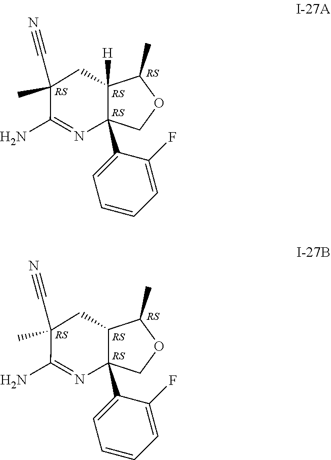 [SMILES: C[C@H]1OC[C@]2(c3ccccc3F)N=C(N)[C@](C)(C#N)C[C@H]12 ; C[C@H]1OC[C@]2(c3ccccc3F)N=C(N)[C@@](C)(C#N)C[C@H]12]

I-26 (7.21 g, 18.6 mmol) was dissolved in formic acid (103 mL) and stirred for 3 h at RT. The solvent was removed in vacuo, the residue was dissolved in DCM and washed with NaHCO₃ sol., the org layer was separated, dried (MgSO₄), filtered and the solvent was removed in vacuo. The reaction mixture was purified with flash column chromatography (DCM/MeOH 100/0 to 96/4). The product fractions were collected and the solvent was removed in vacuo to yield I-27a (1.78 g, 33%) and I-27b (1.75 g, 33%).

Preparation of Intermediate 28a (I-28a)

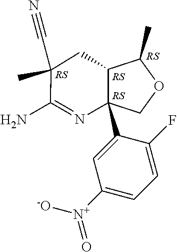

To a stirred solution of I-27a (1.78 g, 6.2 mmol) in TFA (14.222 mL) was added H₂SO₄ (3.30 mL, 62.0 mmol) and the reaction mixture was then cooled down to 0° C. KNO₃ (689.0 mg, 6.8 mmol) was then added and the reaction mixture was stirred at 0° C. for 15 min. DCM was added and at 0° C. the reaction mixture was basified by addition of Na₂CO₃ solution and solid Na₂CO₃ (caution!). The organic layer was separated, dried (MgSO₄), filtered and the solvent was removed in vacuo. The reaction mixture was purified by flash column chromatography (n-heptane/EtOAc 100/0 to 50/50). The product fractions were collected and concentrated in vacuo to yield I-28a (1.29 g, 63%).

Preparation of Intermediate 28b (I-28b)

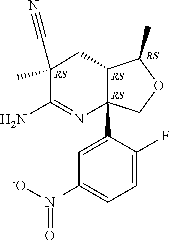

I-28b was prepared according to a procedure analogous to that described for I-28a, starting from I-27b.

Preparation of Intermediate 29 (I-29)

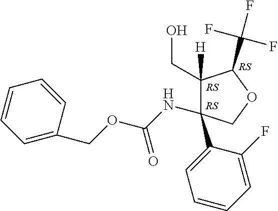 [SMILES: O=C(N[C@@]1(c2ccccc2F)CO[C@H](C(F)(F)F)[C@H]1CO)OCc1ccccc1]

To a stirred solution of 1-5 (8.2 g, 29.4 mmol) in DCM (102.5 mL) was added DIPEA (7.59 mL, 44.1 mmol) and the reaction mixture was stirred under N₂ atmosphere at 0° C. Benzyl chloroformate (4.61 mL, 32.3 mmol) in THF (10 mL) was added dropwise and the reaction mixture was stirred for 5 h at 0° C. Na₂CO₃ sol. and DCM were added, the org layer was separated, dried (MgSO₄), filtered off and evaporated. The residue was suspended from DIPE, the precipitate was filtered off and dried under vacuum at 50° C. to yield I-29 (7.8 g, 64%).

Preparation of Intermediate 30 (I-30)

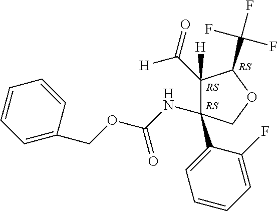 [SMILES: O=C[C@@H]1[C@@H](C(F)(F)F)OC[C@@]1(NC(=O)OCc1ccccc1)c1ccccc1F]

To a stirred solution of I-29 (5.2 g, 12.6 mmol) in DCM (276.6 mL) under N₂ atmosphere at 0° C. was added Dess-Martin periodinane (11.43 g, 18.9 mmol) and the reaction mixture was stirred at 0° C. and allowed to warm up to RT overnight, then it was stirred with a sat. sol. of Na₂S₂O₃. The org layer was separated, washed with a 10% Na₂CO₃ sol. 10% (3×), dried (MgSO₄), filtered and the solvent was evaporated under reduced pressure. The residue was purified by flash column chromatography (silica gel, NP, Biotage flash purification system; n-heptane/EtOAc 100/0 to 50/50). The product fractions were collected and the solvent was evaporated to yield I-30 (4.9 g, 95%).

Preparation of Intermediate 31 (I-31)

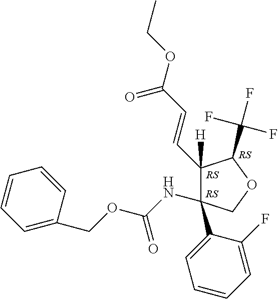 [SMILES: CCOC(=O)/C=C/[C@@H]1[C@@H](C(F)(F)F)OC[C@@]1(NC(=O)OCc1ccccc1)c1ccccc1F]

To a stirred suspension of NaH (60% dispersion in mineral oil, 581 mg, 14.5 mmol) in dry THF (200 mL) under N₂ atmosphere at 0° C. was added dropwise a solution of triethyl phosphonoacetate (3.76 g, 16.8 mmol) in THF (25 mL), with further stirring at 0° C. under N₂ atmosphere for 10 min, followed by dropwise addition of a solution of I-30 (4.60 g, 11.18 mmol) in THF (25 mL), and the reaction mixture was stirred at 0° C. for 30 min. NaHCO₃ sol. was added dropwise, DCM was added, the org layer was separated, dried (MgSO₄), filtered off and the solvent was evaporated under reduced pressure. The residue was purified by flash column chromatography (silica gel, NP, Biotage flash purification system; n-heptane/EtOAc 100/0 to 0/100). The product fractions were collected and the solvent was evaporated to yield I-31 (4.9 g, 91%).

Preparation of Intermediate 32 (I-32)

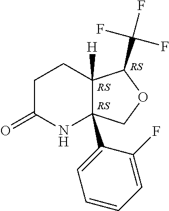 [SMILES: O=C1CC[C@@H]2[C@@H](C(F)(F)F)OC[C@]2(c2ccccc2F)N1]

To a stirred suspension of Pd/C (590 mg, 5.5 mmol) in EtOH (74 mL) under N₂ atmosphere was added a solution of I-31 (5.9 g, 12.3 mmol) in EtOH (74 mL) and the reaction mixture was stirred at RT under H₂ atmosphere (549.5 mL, until 3 eq. H₂ were absorbed). The catalyst was removed by filtration over dicalite®, the solvent was evaporated under reduced pressure, co-evaporated (4×) with CH₃CN at 60° C. and the residue was purified by flash column chromatography (silica gel, NP, Biotage flash purification system; DCM/MeOH 100/0 to 95/5). The product fractions were collected and the solvent was evaporated to yield I-32 (3.5 g, 94%).

Preparation of Intermediate 33 (I-33)

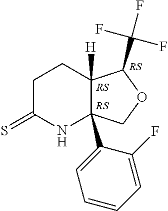 [SMILES: Fc1ccccc1[C@]12CO[C@H](C(F)(F)F)[C@H]1CCC(=S)N2]

Phosphorus pentasulfide (3.59 g, 16.2 mmol) was added to a mixture of I-32 (3.5 g, 11.5 mmol) in THF (93.9 mL) at RT. The mixture was stirred at 70° C. for 3 h. The mixture was cooled and filtered over dicalite® and the solvents evaporated in vacuo. The residue was purified by short column chromatography (heptane/EtOAc 100/0 to 50/50). The desired fractions were collected and concentrated in vacuo to yield I-33 (2.3 g, 62%).

Preparation of Intermediate 34 (I-34)

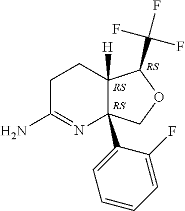 [SMILES: NC1=N[C@@]2(c3ccccc3F)CO[C@H](C(F)(F)F)[C@H]2CC1]

I-33 (1 g, 3.132 mmol) was dissolved in NH₃ (7M in MeOH, 71.43 mL) and the reaction mixture was stirred for 24 h at 80° C. The solvent was evaporated under reduced pressure. The residue was purified by flash column chromatography (silica gel; NP, Biotage flash purification system; eluent DCM/NH₃ 7 M in MeOH 100/0 to 90/10). The product fractions were collected and the solvent was evaporated. The residue was crystalized from DIPE, the precipitate was filtered off and dried under vacuum at 60° C. to yield I-34 (900 mg, 95%).

Preparation of Intermediate 35 (I-35)

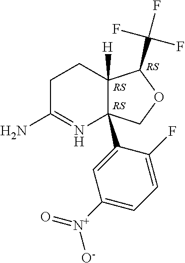 [SMILES: NC1=N[C@@]2(c3cc([N+](=O)[O-])ccc3F)CO[C@H](C(F)(F)F)[C@H]2CC1]

I-34 (900 mL, 3.0 mmol) was stirred in fuming HNO₃ (10 mL) for 1 h. The reaction mixture was poured into ice/water and carefully basified to pH 8 with NaOH 50%. The aq. layer was extracted with DCM, and the org. layer was separated, dried (MgSO₄), filtered and the solvent evaporated to yield I-35 (1 g, 97%).

Preparation of Intermediate 36 (I-36)

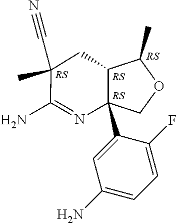 [SMILES: C[C@H]1OC[C@]2(c3cc(N)ccc3F)N=C(N)[C@](C)(C#N)C[C@H]12]

To a stirred suspension of Pd/C (10%, 922.2 mg, 0.9 mmol) in EtOAc (170 mL) under N₂ atmosphere was added a solution of I-28a (1.44 g, 4.333 mmol) in EtOAc (170 mL) and the reaction mixture was stirred for 7 h at RT under H₂ atmosphere. The catalyst was filtered off over dicalite under N₂ atmosphere and the solvent was removed in vacuo to yield I-36 (1.31 g, quant.) which was used without further purification.

The following intermediates were prepared in an analogous manner from the indicated starting material:

| STARTING | |
|---|---|
| MATERIAL | INTERMEDIATE |
| I-35 | 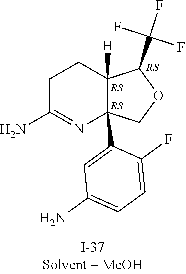 [SMILES: NC1=N[C@@]2(c3cc(N)ccc3F)CO[C@H](C(F)(F)F)[C@H]2CC1] |
| I-28b | 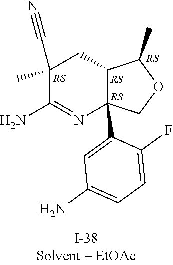 [SMILES: C[C@H]1OC[C@]2(c3cc(N)ccc3F)N=C(N)[C@@](C)(C#N)C[C@H]12] |

Preparation of Intermediate 39 (I-39)

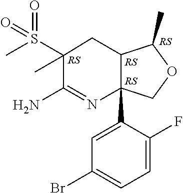 [SMILES: C[C@H]1OC[C@]2(c3cc(Br)ccc3F)N=C(N)C(C)(S(C)(=O)=O)CC12]

I-21 (300 mg, 0.6 mmol) was dissolved in formic acid (7.6 mL, 201.6 mmol) and the reaction mixture was heated overnight at 80° C. The solvent was evaporated and the residue was dissolved in DCM. 10% Na₂CO₃ solution was added (aq. layer should be basic). The org layer was separated, dried (MgSO₄), filtered off and the solvent was evaporated under reduced pressure to afford a crude that was purified by column chromatography (silica gel; DCM/7N NH₃ in MeOH 100/0 to 98/2) to afford I-39 (180 mg, yield 74%) as a 1:2 diastereomeric mixture.

Preparation of Intermediate 40 (I-40)

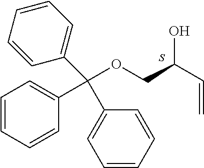

A solution of trimethylsulfonium iodide ([2181-42-2], 38.7 g, 189.64 mmol) in THF (246.12 mL) was stirred for 1 h at rt under N₂. The mixture was cooled to −60° C. and nBuLi (2.5 M, 78.86 mL, 189.84 mmol) was added slowly (addition funnel). The r.m. was allowed to warm to −30° C. and stirred for 1 h. (2S)-2-trityloxymethyl oxirane ([129940-50-7], 20 g, 63.21 mmol) was added portionwise and the r.m. was stirred at rt for 2 h. The r.m. was poured over NH₄Cl (sat), the layers were partitioned, the aqueous layer was extracted with EtOAc, the combined organic layers were dried (MgSO₄), filtered and concentrated under reduced pressure. The crude was purified using flash chromatography (SiO₂; EtOAc:heptane 2-5%) to yield I-40 (20.8 g, 99%, 99% purity).

Preparation of Intermediate 41 (I-41)

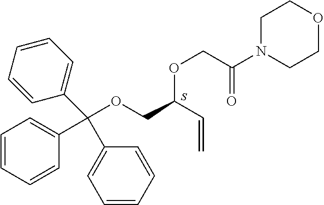

To a solution of 1-40 (15.31 g, 46.34 mmol) in toluene (104.25 mL) at 5° C. were added tetrabutylammonium hydrogen sulfate (1.57 g, 4.63 mmol), 4-(2-chloroacetyl) morpholine (9.35 mL, 71.82 mmol) and a solution of NaOH (18.53 g, 463.35 mmol) in water (25 mL). The r.m. was stirred for 19 h, while allowing the temperature to reach rt, then water (15 mL) and toluene (5 mL) were added. The layers were separated and the organic layer was washed with water and brine. The organic layer was dried (MgSO₄), filtered and the solvent was removed under reduced pressure to yield I-41 (19.3 g, 91%) as a solid, which was used in the subsequent step without further purification.

Preparation of Intermediate 42 (I-42)

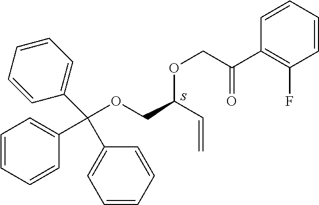

To a stirred solution of 2-fluoroiodobenzene (11.96 mL, 102.54 mmol) in toluene (100 mL) at 5° C. (ice bath) was added iPrMgCl.LiCl (78.19 mL, 101.65 mmol) and the solution was stirred for 1 h at 5° C. I-41 (20.4 g, 44.58 mmol) was dissolved in toluene (200 mL) and cooled to 5° C. 90% of the 2-fluoroiodobenzene mixture was added and the mixture was stirred for 2 h at 5° C., followed by the remaining 10% 2-fluoroiodobenzene mixture and stirring was maintained for an additional 1 h.

When the reaction was finished, citric acid 1M was added (250 mL) at 5° C., the layers were separated and the organic layer was washed with water (25 mL), dried (MgSO₄), filtered and the solvents were removed under reduced pressure. The crude was dissolved in MeOH (100 mL) and the solvent was evaporated to yield I-42 (25.32 g, 99% purity), which was used in the subsequent step without further purification.

Preparation of Intermediate 43 (I-43)

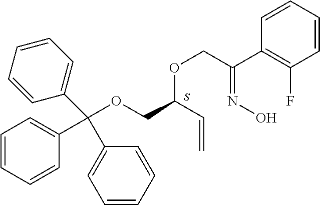

To a solution of I-42 (18.61 g, 39.9 mmol) in MeOH (160 mL), were added NaOAc (7.20 g, 87.76 mmol) and NH₂OH.HCl (4.16 g, 59.83 mmol) and the r.m. was heated for 2 h at 50° C. The solvent was then evaporated and the residue was dissolved in water and toluene. The layers were separated and the aqueous layer was extracted with toluene. The combined organic layers were washed with water, dried (MgSO₄), filtered and the solvent was evaporated in vacuo to obtain I-43 (19.26 g, 99% purity, quantitative), which was used in the subsequent step without further purification.

Preparation of Intermediate 44 (I-44)

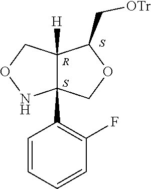

A solution of I-43 (19.26 g, 40 mmol) and hydroquinone (4.49 g, 40.80 mmol) in toluene was heated to reflux under nitrogen for 24 h. The solution was cooled to rt and sodium carbonate was added. The layers were separated and the aqueous was extracted with toluene. The combined organic layers were washed with water, dried (MgSO₄), filtered and the solvent was removed under reduced pressure. Isopropyl alcohol was added and the mixture was heated to reflux, cooled to rt and the solid was filtered and dried under vacuum. Two fractions of I-44 (10.18 g and 2.95 g, 67%) were obtained.

Preparation of Intermediate 45 (I-45)

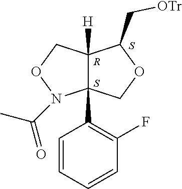

To a solution of I-44 (10.18 g, 21.14 mmol) in DCM (63.42 mL) at to 0° C. were added DMAP (0.35 g, 2.85 mmol) and pyridine (3.15 mL, 39.11 mmol). Acetyl chloride (1.81 mL, 25.37 mmol) was added slowly and the r.m. was allowed to reach rt during 1 h. The r.m. was cooled to 0° C. and the same amount of DMAP and pyridine, followed by the same amount of acetyl chloride were added slowly. The r.m. was allowed to reach rt during 1 h. When the reaction was complete, the r.m. was cooled and water was added and stirred for 30 min. The layers were separated and the aqueous was extracted with DCM. Combined organic layer were washed with HCl (1M), and the aqueous layer was extracted with DCM. The combined organic layers were washed with water, dried (MgSO₄), filtered and the solvent was evaporated under reduced pressure to yield I-45 (7.52 g, 67%, 99% purity).

Preparation of Intermediate 46 (I-46)

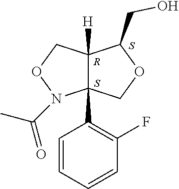

To a solution of I-45 (19.78 g, 37.78 mmol) in DCM (132.22 mL) under N₂ was added formic acid (20.67 mL, 547.77 mmol) and the r.m. was stirred for 4 days.

When the reaction was complete the solvent was removed under reduced pressure and MeOH and Na₂CO₃ (sat+solid) were added until pH=8. The reaction mixture was heated at 45° C. (55° C. bath) for 45 min. The r.m. was cooled to rt, NaOH 2M was added and stirred for 1 h at rt. After this, the solvent was removed under reduced pressure and the crude was partitioned between EtOAc and water. The layers were separated and the aqueous was extracted with EtOAc. The combined organic layers were washed with brine, dried (MgSO₄), filtered and the solvents were removed under reduced pressure. The crude was purified using flash chromatography (SiO₂; MeOH:DCM 20-60%) to yield I-46 (6.36 g, 60%) as a white solid. Additional fractions were crystallized from toluene to yield additional I-46 (1.44 g, 14%) as a white solid.

I-46 can also be purified by trituration with methyl tert-butyl ether.

Preparation of Intermediate 47 (I-47)

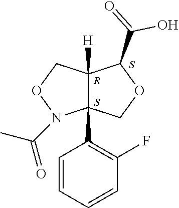

To a mixture of I-46 (4 g, 14.72 mmol) in ACN (32 mL) and H₂O (25 mL) at 0° C., were added TEMPO (223.63 mg, 1.42 mmol) and (diacetoxyiodo)benzene (10.53 g, 32.71 mmol) portionwise keeping the temperature below 5° C. The reaction mixture was stirred until full conversion. A mixture of sodium thiosulfate (4.1 g) and K₂CO₃ (4.2 g) solution in H₂O (25 mL) was added carefully at 0° C., and stirred for 1 h at RT. The aqueous layer was cooled down on an ice-bath, HCl conc. was added carefully (until acidic pH, around 3) and DCM was added, the organic layer was separated, dried and the solvent was evaporated under reduced pressure to yield I-47 (3 g, 71%).

Preparation of Intermediate 48 (I-48)

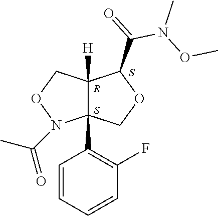

To a stirred solution of I-47 (3 g, 10.16 mmol) in DMF (100 mL) at 0° C., was added CDI (4.94 g, 30.48 mmol) and the reaction mixture was stirred for 2 h at RT. Et₃N (9.18 mL, 66.04 mmol) and N,O-dimethylhydroxylamine hydrochloride (3.47 g, 35.56 mmol) were then added and the reaction mixture was stirred overnight. The solvent was evaporated under reduced pressure. DCM and a Na₂CO₃ solution (10% in H₂O) were added. The organic layer was separated, dried (MgSO₄), filtered and the solvent was evaporated under reduced pressure.

The residue was purified by column chromatography (silicagel; eluent: 100% n-heptane to 100% EtOAc). The desired fractions were collected and the solvent was evaporated under reduced pressure to obtain I-48 (4 g, 31% purity), which was used without further purification.

Preparation of Intermediate 49 (I-49)

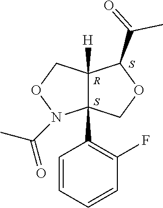

To a stirred solution of I-48 (31% purity by LCMS; 4 g, 11.82 mmol) in dry THF (60 mL) under N₂ atmosphere at −60° C. (CO₂/iPrOH bath) was added methylmagnesium bromide (5.22 mL, 17.73 mmol) dropwise and the reaction mixture was stirred for 1 h at −40° C., then it was allowed to warm up to −20° C. NH₄Cl solution was added dropwise, DCM was added, and the organic layer was separated, dried (MgSO₄), filtered off and the solvent was evaporated under reduced pressure.

The residue was suspended from n-heptane, filtered and dried under vacuum at 50° C. to yield I-49 (1.4 g, 40%).

Preparation of Intermediate 50 (I-50)

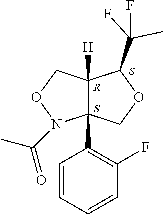

To a stirred solution of I-49 (1.11 g, 3.77 mmol) in dry DCE (10 mL) at 0° C. under N₂ atmosphere, was added Xtalfluor-M® (2.75 g, 11.31 mmol) in portions, and the reaction mixture was stirred for 10 min. Trimethylamine hydrofluoride (1.23 mL, 7.54 mmol) was added dropwise, the reaction mixture was stirred for 4 h at 0° C. and allowed slowly to warm to RT overnight while stirring under N₂ atmosphere. NaOH (50% aq. sol., 1.5 mL) was slowly added while maintaining the temperature under 10° C., additional H₂O (2.5 mL), NaHCO₃ aq. sol. (2.5 mL), and DCM were added. The organic layer was separated, the aqueous layer was extracted with DCM, and the combined organic layers were dried (MgSO₄), filtered off and the solvent was evaporated under reduced pressure. The resulting residue was purified by column chromatography (silica gel; gradient n-heptane/EtOAc 100/0 to 0/100). The desired fractions were collected and the solvent was evaporated under reduced pressure to yield I-50 (800 mg, 67%).

Preparation of Intermediate 51 (I-51)

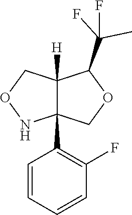 [SMILES: CC(F)(F)[C@H]1OC[C@]2(c3ccccc3F)NOC[C@H]12]

To a stirred solution of I-50 (800 mg, 2.54 mmol) in 1,4-dioxane (12 mL), was added HCl (37% in H₂O, 3.18 mL, 38.1 mmol) and the reaction mixture was stirred for 3 h at 100° C. The reaction mixture was then cooled down below 10° C., and NaOH (50% aq. sol.) and EtOAc were added. The organic layer was separated, washed with brine, dried (MgSO₄), filtered off and the solvent was evaporated under reduced pressure, to yield I-51 (700 mg, quant.) which was used without further purification.

The following intermediates were prepared in an analogous manner from the indicated starting material

| STARTING | |
|---|---|
| MATERIAL | INTERMEDIATE |
| I-73 | 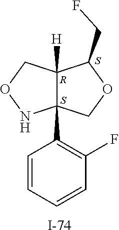 |

Preparation of Intermediate 52 (I-52)

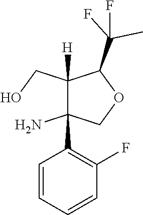 [SMILES: CC(F)(F)[C@H]1OC[C@@](N)(c2ccccc2F)[C@@H]1CO]

To a stirred solution of I-51 (4 g, 14.64 mmol) in AcOH (114.29 mL) was added Zinc (7.66 g, 117.11 mmol) and the reaction mixture was stirred overnight at RT, then it was filtered through dicalite®, and rinsed with MeOH. The solvent was evaporated under reduced pressure. DCM and Na₂CO₃ sat. sol. were added, the organic layer was separated, dried (MgSO₄), filtered off and the solvent was evaporated under reduced pressure to yield I-52 (3.98 g, 99%) which was used without further purification.

The following intermediates were prepared in an analogous manner from the indicated starting material

| STARTING | |
|---|---|
| MATERIAL | INTERMEDIATE |
| I-74 | 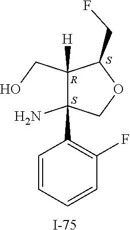 |
| I-86 | 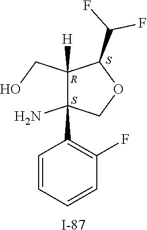 |

Preparation of Intermediate 53 (I-53)

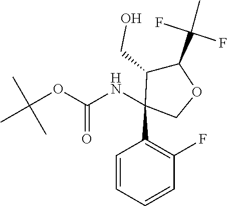 [SMILES: CC(C)(C)OC(=O)N[C@@]1(c2ccccc2F)CO[C@H](C(C)(F)F)[C@H]1CO]

To a stirred solution of I-52 (4.5 g, 16.35 mmol) in MeOH (150 mL), was added Boc₂O (7.14 g, 32.70 mmol) and the reaction mixture was stirred overnight at 50° C. The solvent was evaporated under reduced pressure, and DCM and Na₂CO₃ sol. were added. The organic layer was separated, dried (MgSO₄), filtered off and the solvent was evaporated under reduced pressure. The resulting product was purified with column chromatography (silica gel; gradient DCM/MeOH 100/0 to 92.5/7.5) to yield I-53 (4.5 g, 73%).

The following intermediates were prepared in an analogous manner from the indicated starting material

| STARTING | |
|---|---|
| MATERIAL | INTERMEDIATE |
| I-75 | 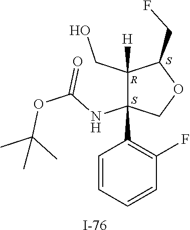 |
| I-87 | 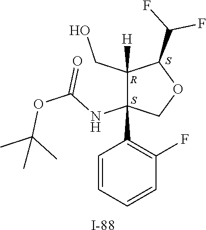 |

Preparation of Intermediate 54 (I-54)

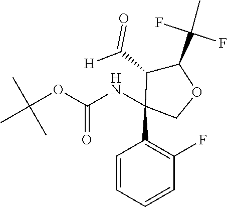 [SMILES: CC(C)(C)OC(=O)N[C@@]1(c2ccccc2F)CO[C@H](C(C)(F)F)[C@H]1C=O]

Dess-Martin periodinane (4.96 g, 11.48 mmol) was added portionwise to a solution of I-53 (2.8 g, 7.46 mmol) in DCM (218 mL). The reaction was stirred at rt for 3 h, then treated with Na₂SO₃ sat. sol. and stirred 10 min at rt. The organic phase was then separated and washed with Na₂CO₃ solution (2 times) and the aqueous layer was extracted with DCM. The combined organic layers were dried (MgSO₄), filtered and evaporated. The residue was purified by flash column chromatography (silica gel; EtOAc in heptane 0/100 to 40/60). The desired fractions were collected and evaporated in vacuo to yield I-54 (2.7 g, 97%).

The following intermediates were prepared in an analogous manner from the indicated starting material

| STARTING | |
|---|---|
| MATERIAL | INTERMEDIATE |
| I-76 | 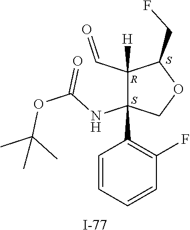 |
| I-88 | 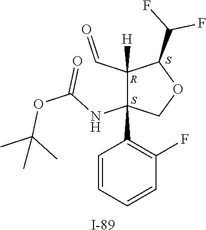 |

Preparation of Intermediate 55 (I-55)

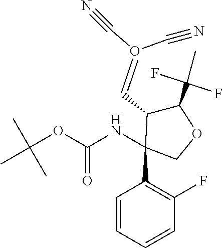

To a stirred solution of I-54 (2.7 g, 7.23 mmol) in MeOH (37.34 mL), were added malononitrile (1.72 g, 26.08 mmol), Magnesium oxide (349.75 mg, 8.68 mmol) and Ti(i-PrO)4 (4.23 mL, 14.46 mmol), and the mixture was stirred at 65° C. for 30 min. H₂O and DCM were added, the white precipitate was filtered off over dicalite. The organic layer was separated, dried (MgSO₄), filtered off and the solvent was evaporated to yield I-55 (3.04 g, quant.), which was used without further purification.

The following intermediates were prepared in an analogous manner from the indicated starting material

| STARTING | |
|---|---|
| MATERIAL | INTERMEDIATE |
| I-77 | 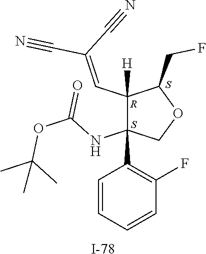 |

Preparation of Intermediate 56 (I-56)

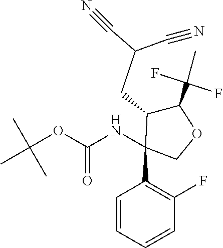 [SMILES: CC(C)(C)OC(=O)N[C@@]1(c2ccccc2F)CO[C@H](C(C)(F)F)[C@H]1CC(C#N)C#N]

To a stirred solution of I-55 (870 mg, 2.06 mmol) in THF (20 mL) at 0° C., was added NaBH₄ (85.91 mg, 2.27 mmol) and the reaction mixture was stirred for 30 min at 0° C. Na₂CO₃ sol. and DCM were added, the organic layer was separated, dried (MgSO₄), filtered off and the solvent was evaporated under reduced pressure. The residue was purified by column chromatography (silica gel; gradient n-heptane/EtOAc 100/0 to 0/100), the desired product fractions were collected and the solvent was evaporated under reduced pressure to yield I-56 (700 mg, 80%).

The following intermediates were prepared in an analogous manner from the indicated starting material

| STARTING | |
|---|---|
| MATERIAL | INTERMEDIATE |
| I-78 | 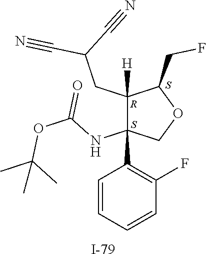 |

Preparation of Intermediate 57 (I-57)

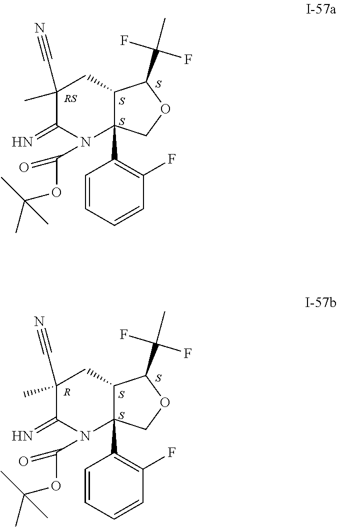

To a stirred solution of I-56 (700 mg, 1.65 mmol) in dry THF (28 mL) under N₂ atmosphere at 0° C. was added NaH (60% dispersion in mineral oil, 92.57 mg, 2.31 mmol) and the mixture was stirred for 15 min at 0° C. under a N₂ atmosphere. CH₃I (144.08 μL, 2.31 mmol) was added, and the mixture was further stirred for 3 h at 0° C. NH₄Cl sat. sol. and DCM were added, the organic layer was separated, dried (MgSO₄), filtered off and the solvent was evaporated under reduced pressure. The residue was purified by column chromatography (silica gel; gradient n-heptane/EtOAc 100/0 to 0/100). The product fractions were collected and the solvent was evaporated under reduced pressure to yield I-57a (328 mg, 45%, containing an impurity) and I-57b (280 mg, 38%).

The following intermediates were prepared in an analogous manner from the indicated starting material

| STARTING | |
|---|---|
| MATERIAL | INTERMEDIATE |
| I-90 | 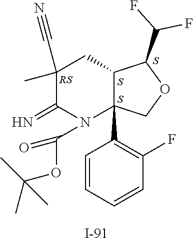 |

Preparation of Intermediate 58 (I-58a)

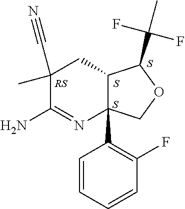

I-57a (328 mg, 0.75 mmol) was stirred in formic acid (10 mL) at room temperature for 3 h. The solvent was evaporated under reduced pressure, DCM and Na₂CO₃ solution were added. The organic layer was separated, dried (MgSO₄), filtered and the solvent was evaporated to yield I-58a (215 mg, 85%).

The following intermediates were prepared in an analogous manner from the indicated starting material

| STARTING | |
|---|---|
| MATERIAL | INTERMEDIATE |
| I-91 | 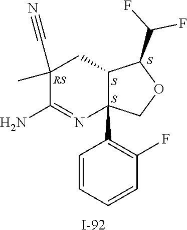 |

Preparation of Intermediate 59 (I-59)

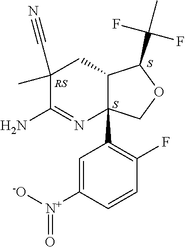

To a solution of I-58 (215 mg, 0.64 mmol) in TFA (6.24 mL) at 0° C., was added H₂SO₄ (509.6 μL, 9.56 mmol), followed by KNO₃ (80.55 mg, 0.80 mmol), giving a yellow solution. After 30 min stirring, the reaction mixture was poured into an ice/NH₃/DCM mixture. The organic layer was separated and the aqueous layer was extracted with DCM. The combined organic layers were dried (MgSO₄), filtered and the filtrate was concentrated in vacuo to yield I-59 (240 mg, 98%), which was used as such in the following step.

The following intermediates were prepared in an analogous manner from the indicated starting material

| STARTING | |
|---|---|
| MATERIAL | INTERMEDIATE |
| I-92 | 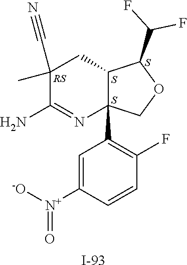 |

Preparation of Intermediate 60 (I-60)

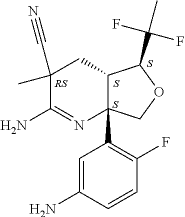

To a solution of I-59 (240 mg, 0.628 mmol) in MeOH (7.0 mL) and water (1.5 mL), were added iron (280.45 mg, 5.02 mmol) and NH₄Cl (364.72 mg, 6.82 mmol) and the reaction mixture was stirred at 70° C. for 1 h. The reaction mixture was then cooled to room temperature, MeOH and DCM were added, and the mixture was filtered over dicalite®. The organic layer was washed with water, dried (MgSO₄), filtered and concentrated to yield I-60 (180 mg, 81%), which was used without further manipulation.

The following intermediates were prepared in an analogous manner from the indicated starting material

| STARTING | |
|---|---|
| MATERIAL | INTERMEDIATE |
| I-93 | 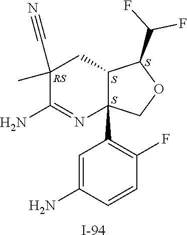 |

Preparation of Intermediate 61 (I-61)

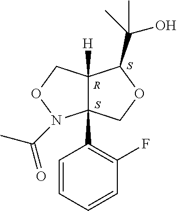

To a stirred solution of I-49 (5.57 g, 18.03 mmol) in dry THF (110 mL) at −10° C. under N₂ atmosphere, was added Methyl magnesium bromide (1.4M in Toluene/THF, 14.17 ml, 19.83 mmol). The reaction mixture was stirred for 1 hour at 0° C. The reaction mixture was carefully quenched with saturated NH₄Cl. The reaction mixture was extracted with MTBE, the organic layer was separated, dried over MgSO₄, filtered off and the filtrate was evaporated under reduced pressure. The resulting residue was purified by column chromatography (silica gel; gradient n-heptane/EtOAc 100/0 to 60/40). The desired fractions were collected and the solvent was evaporated under reduced pressure to yield I-61 (4500 mg, 81%) as a white solid.

Preparation of Intermediate 62 (I-62)

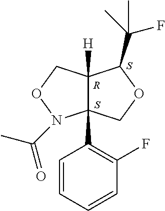

To a stirred solution of I-61 (2.15 g, 6.95 mmol) in DCM (34 mL) at −10° C. under N₂ atmosphere, was added DAST (2.16 ml, 16.33 mmol). The reaction mixture was stirred for 4 hours at room temperature. The reaction mixture was carefully quenched with saturated NaHCO₃ solution. The reaction mixture was extracted with DCM, the organic layer was separated, dried over MgSO₄, filtered off and the filtrate was evaporated under reduced pressure to yield I-62 (1125 mg, 52%).

Preparation of Intermediate 63 (I-63)

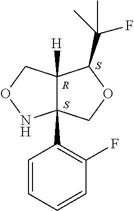

To a stirred solution of I-62 (2.31 g, 7.43 mmol) in 1,4-dioxane (26 mL), was added HCl (37% in H₂O, 6.67 mL, 80.06 mmol) and the reaction mixture was stirred for 3 h at 100° C. The reaction mixture was then cooled down below 10° C., and NaOH (50% aq. sol., 6.4 ml, 80.06 mmol) and EtOAc were added. The organic layer was separated, washed with brine, dried (MgSO₄), filtered off and the solvent was evaporated under reduced pressure, to yield I-63 (1.79 g, purity 60%, 54%) which was used without further purification.

Preparation of Intermediate 64 (I-64)

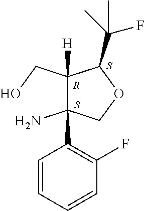

To a stirred solution of I-63 (1.79 g, 6.66 mmol) in AcOH (50 mL) was added Zinc (3 g, 45.8 mmol) and the reaction mixture was stirred overnight at RT, then it was filtered through dicalite®, and rinsed with MeOH. The solvent was evaporated under reduced pressure. DCM and Na₂CO₃ sat. sol. were added, the organic layer was separated, dried (MgSO₄), filtered off and the solvent was evaporated under reduced pressure to yield I-64 (1.66 g, purity 60%, 55%) which was used without further purification.

Preparation of Intermediate 65 (I-65)

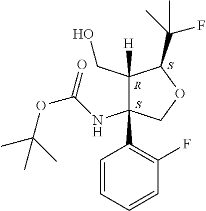

To a stirred solution of I-64 (1.66 g, 6.11 mmol) in MeOH (56 mL), was added Boc₂O (2.67 g, 12.2 mmol) and the reaction mixture was stirred overnight at 50° C. The solvent was evaporated under reduced pressure, and DCM and Na₂CO₃ sol. were added. The organic layer was separated, dried (MgSO₄), filtered off and the solvent was evaporated under reduced pressure. The resulting product was purified with column chromatography (silica gel; gradient n-heptane/EtOAc 100/0 to 0/100) to yield I-65 (0.67 g, 29%).

Preparation of Intermediate 66 (I-66)

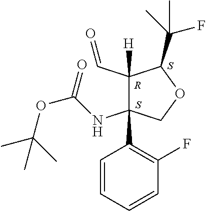

Dess-Martin periodinane (1.28 g, 2.83 mmol) was added portionwise to a solution of I-65 (0.67 g, 1.8 mmol) in DCM (53 mL). The reaction was stirred at rt for 1 h, then treated with a solution of NaHCO₃ until pH 8 and Na₂SO₃ sat. sol. and stirred 30 min at rt. The organic phase was then separated and washed with Na₂CO₃ solution (3 times) and the aqueous layer was extracted with DCM. The combined organic layers were dried (MgSO₄), filtered and evaporated. The residue was purified by flash column chromatography (silica gel; EtOAc in heptane 0/100 to 100/0). The desired fractions were collected and evaporated in vacuo to yield I-66 (0.58 g, 78%).

Preparation of Intermediate 67 (I-67)

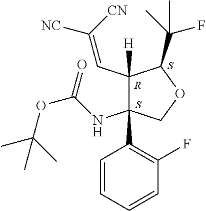

To a stirred solution of I-66 (0.58 g, 1.57 mmol) in MeOH (8 mL), were added malononitrile (0.37 g, 5.67 mmol), Magnesium oxide (76 mg, 1.88 mmol) and Ti(i-PrO)₄ (0.92 mL, 3.14 mmol), and the mixture was stirred at 65° C. for 40 min. H₂O and DCM were added, the white precipitate was filtered off over dicalite®. The organic layer was separated, dried (MgSO₄), filtered off and the solvent was evaporated to yield I-67, which was used without further purification.

Preparation of Intermediate 68 (I-68)

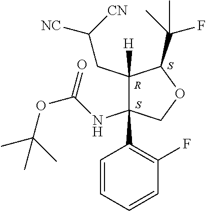

To a stirred solution of I-67 (655 mg, 1.57 mmol) in THF (15 mL) at 0° C., was added NaBH₄ (65 mg, 1.72 mmol) and the reaction mixture was stirred for 30 min at 0° C. Na₂CO₃ sol. and DCM were added, the organic layer was separated, dried (MgSO₄), filtered off and the solvent was evaporated under reduced pressure. The residue was purified by column chromatography (silica gel; gradient n-heptane/EtOAc 100/0 to 0/100), the desired product fractions were collected and the solvent was evaporated under reduced pressure to yield I-68 (518mg, 60%).

Preparation of Intermediate 69 (I-69)

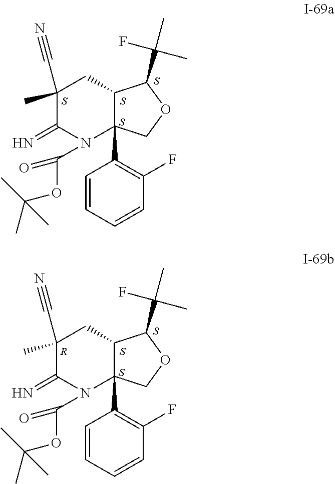

To a stirred solution of I-68 (518 mg, 1.23 mmol) in dry THF (21 mL) under N₂ atmosphere at 0° C. was added NaH (60% dispersion in mineral oil, 69.15 mg, 1.73 mmol) and the mixture was stirred for 15 min at 0° C. under a N₂ atmosphere. CH₃I (245 mg, 1.73 mmol) was added, and the mixture was further stirred for 1 h at 0° C. NH₄Cl sat. sol. and DCM were added, the organic layer was separated, dried (MgSO₄), filtered off and the solvent was evaporated under reduced pressure. The residue was purified by column chromatography (silica gel; gradient n-heptane/EtOAc 100/0 to 0/100). The product fractions were collected and the solvent was evaporated under reduced pressure to yield I-69a (196 mg, 37%) and I-69b (226 mg, 42%).

Preparation of Intermediate 70 (I-70a)

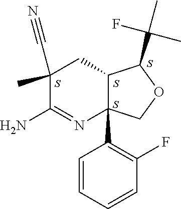

I-69a (141 mg, 0.32 mmol) was stirred in formic acid (4.3 mL) at room temperature for 3 h. The solvent was evaporated under reduced pressure, DCM and Na₂CO₃ solution were added. The organic layer was separated, dried (MgSO₄), filtered and the solvent was evaporated to yield I-70a (117 mg, 94%).

The following intermediates were prepared in an analogous manner from the indicated starting material

| STARTING | |
|---|---|
| MATERIAL | INTERMEDIATE |
| I-69b | 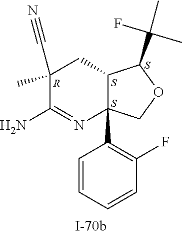 |
| I-80a | 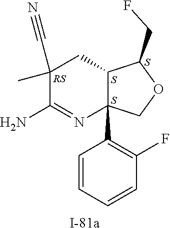 |
| I-80b | 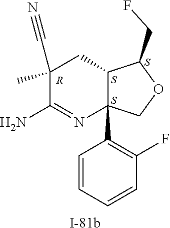 |

Preparation of Intermediate 71 (I-71a)

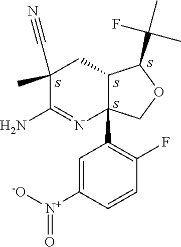

To a stirred solution of I-70a (117 mg, 0.305 mmol) in TFA (3 mL) at 0° C., was added H₂SO₄ (450 mg, 4.58 mmol), followed by KNO₃ (38.6 mg, 0.382 mmol), giving a yellow solution. After 30 min stirring, the reaction mixture was poured into an ice/NH₃/DCM mixture. The organic layer was separated and the aqueous layer was extracted with DCM. The combined organic layers were dried (MgSO₄), filtered and the filtrate was concentrated in vacuo. The residue was purified by column chromatography (silica gel; gradient n-heptane/EtOAc 100/0 to 0/100). The product fractions were collected and the solvent was evaporated under reduced pressure to yield I-71a (74 mg, 64%).

The following intermediates were prepared in an analogous manner from the indicated starting material

| STARTING | |
|---|---|
| MATERIAL | INTERMEDIATE |
| I-70b | 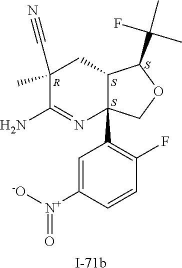 |
| I-81a | 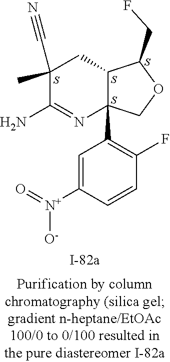 |
| I-81b | 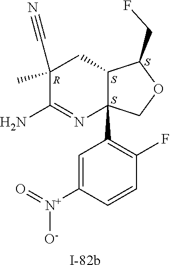 |

Preparation of Intermediate 72a (I-72a)

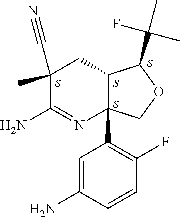

To a solution of I-71a (74 mg, 0196 mmol) in MeOH (2.2 mL) and water (0.468 mL), were added iron (87.37 mg, 1.56 mmol) and NH₄Cl (113.63 mg, 2.12 mmol) and the reaction mixture was stirred at 70° C. for 1 h. The reaction mixture was then cooled to room temperature, MeOH and DCM were added, and the mixture was filtered over dicalite®. The organic layer was washed with water, dried (MgSO₄), filtered and concentrated to yield I-72a (180 mg, 81%), which was used without further manipulation.

The following intermediates were prepared in an analogous manner from the indicated starting material

| STARTING | |
|---|---|
| MATERIAL | INTERMEDIATE |
| I-71b | 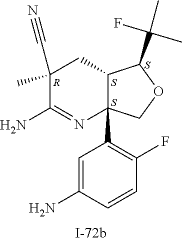 |
| I-82a | 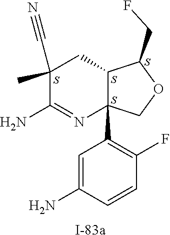 |
| I-82b | 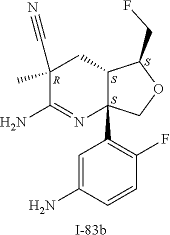 |

Preparation of Intermediate 73 (I-73)

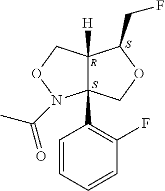

To a stirred solution of I-46 (2.5 g, 8.89 mmol) in THF (120 mL) at 0° C. under N₂ atmosphere, was added Deoxo-Fluor® 50% in toluene (11.79 g). The reaction mixture was stirred for 9 hours at 40° C. The reaction mixture was diluted with EtOAc and carefully quenched with saturated NaHCO₃ solution. The organic phase was separated and the aq. layer was extracted with EtOAc. The combined organic layers were dried over MgSO₄, filtered off and the filtrate was evaporated under reduced pressure. The residue was purified by column chromatography (silica gel; gradient n-heptane/EtOAc 95/5 to 60/40). The product fractions were collected and the solvent was evaporated under reduced pressure to yield I-73 (2.1 g, 83%).

Preparation of Intermediate 80 (I-80)

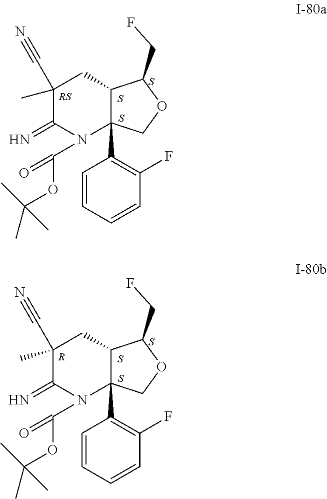

To a stirred solution of I-79 (1.4 g, 3.58 mmol) in dry THF (60 mL) under N₂ atmosphere at 0° C. was added NaH (60% dispersion in mineral oil, 200.28 mg, 5.0 mmol) and the mixture was stirred for 30 min at 0° C. under a N₂ atmosphere.

CH₃I (710.76 mg, 5.0 mmol) was added, and the mixture was further stirred for 3 h at 0° C. NH₄Cl sat. sol. and DCM were added, the organic layer was separated, dried (MgSO₄), filtered off and the solvent was evaporated under reduced pressure. The residue was purified by column chromatography (silica gel; gradient n-heptane/EtOAc 100/0 to 0/100). The product fractions were collected and the solvent was evaporated under reduced pressure to yield I-80a (659 mg) and I-80b (395 mg).

Preparation of Intermediate 84 (I-84)

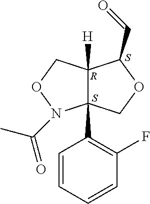

To a mixture of 1-46 (5.78 g, 20.549 mmol) in DCM (150 mL) at 0° C., was added Dess-Martin periodinane (10.46 g, 24.66 mmol). The reaction mixture was stirred until full conversion at RT. Sodium thiosulfate solution 10% was added and the reaction mixture was stirred for 30 minutes then NaHCO₃ solution was added carefully at 0° C., and stirred for 15 minutes at RT. The organic layer was separated and washed 3 times with a Na₂CO₃ solution. The organic layer was separated, dried with MgSO₄ and the solvent was evaporated under reduced pressure to yield I-84 (5.7 g, 99%) which was used without further purification.

Preparation of Intermediate 85 (I-85)

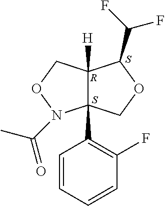

To a mixture of I-84 (5 g, 17.90 mmol) in DCM (150 mL) at 0° C., was added dropwise DAST (10.13 ml, 71.62 mmol). The reaction mixture was stirred overnight at RT. To the reaction mixture was added dropwise 0.150 ml of MeOH at 0° C. The reaction mixture was further decomposed carefully with 10% NaHCO₃ solution keeping the temperature below 5° C. DCM was added and the organic layer was separated, dried with MgSO₄ and the solvent was evaporated under reduced pressure. The residue was purified by column chromatography (silica gel; gradient n-heptane/EtOAc 100/0 to 0/100). The product fractions were collected and the solvent was evaporated under reduced pressure to yield I-85 (2.72 g, 50%).

Preparation of Intermediate 86 (I-86)

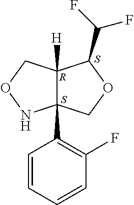

To a mixture of I-85 (2.6 g, 8.63 mmol) in dry THF (41 mL) at −45° C. under N₂ atmosphere, was added dropwise DIBAL 1M in heptane (12.95 ml, 12.95 mmol) at temperature below −40° C. The reaction mixture was stirred for 1 hour at −50° C. To the reaction mixture was added dropwise 0.5 ml of MeOH. Then it was allowed to warm up to −30° C. and a NH₄Cl solution 10% was added dropwise. The reaction mixture was allowed to warm up to rt. DCM was added and the organic layer was separated, dried with MgSO₄ and the solvent was evaporated under reduced pressure. The residue was purified by column chromatography (silica gel; gradient n-heptane/EtOAc 100/0 to 50/50). The product fractions were collected and the solvent was evaporated under reduced pressure to yield I-86 (1.5 g, 67%).

Preparation of Intermediate 90 (I-90

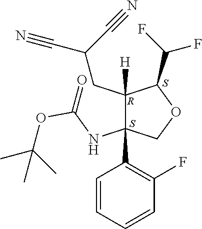

To a stirred solution of I-89 (2.15 g, 5.98 mmol) in MeOH (31 mL), were added malononitrile (1.42 g, 21.58 mmol), Magnesium oxide (289.37 mg, 7.18 mmol) and Ti(i-PrO)4 (3.5 mL, 11.96 mmol), and the mixture was stirred at 65° C. for 80 min. The reaction mixture was cooled to 0° C., THF was added (264 ml). At 0° C., NaBH₄ (249 mg, 6.58 mmol) was added and the reaction mixture was stirred for 30 min at 0° C. Then NaHCO₃ sol. was added, the reaction mixture was stirred at room temperature. dicalite® was added and the reaction mixture was filtered over a layer of dicalite®. The dicalite® layer was washed with DCM. To the filtrate was added DCM and the organic layer was seperated. The aq. layer was extracted twice with DCM, the combined organic layers were dried (MgSO₄), filtered off and the solvent was evaporated under reduced pressure. The residue was purified by column chromatography (silica gel; gradient n-heptane/EtOAc 100/0 to 50/50), the desired product fractions were collected and the solvent was evaporated under reduced pressure to yield I-90 (2.27 g, 93%).

Preparation of Intermediate 95 (I-95)

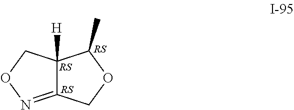

To a stirred solution of 2-(1-methylallyloxy)acetaldehyde oxime [1174321-58-4] (30 g, 232.28 mmol) in DCM (300 ml) at 0° C. was added dropwise NaOCl (7.5% solution in H₂O, 460 ml), and the reaction mixture was stirred for 1 hour at 0° C. DCM (500 ml) was added And the organic layer was separated and dried over anhydrous Na₂SO₄, filtered and the filtrate was concentrated under reduced pressure to afford I-95 (25 g, 85%) as a crude product.

Preparation of Intermediate 96 (I-96)

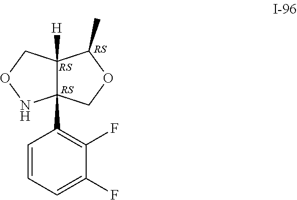

A solution of nBuLi (2.5 M in hexanes, 95.64 mL, 239.10 mmol) was added dropwise to a solution containing bromo-2,3-difluorobenzene (46.15 g, 239.11 mmol) in dry THF (300 ml) under a N₂ atmosphere at −78° C. The reaction mixture was stirred for 30 min at −78° C. A solution of I-95 (19 g, 119.55 mmol) in dry THF (50.0 mL) was added dropwise, and after stirring at −78° C. for 1 h, aqueous NH₄Cl (150 ml) was added, followed by warming to RT. H₂O (100 ml) and EtOAc (100 ml) were added, the organic layer was seperated and the water layer was extracted 3 times with EtOAc (300 ml). The combined organic portions were dried (MgSO₄), evaporated, and the residue was purified by column chromatography (silica gel; petroleum ether/ethyl acetate=20:1 to petroleum ether/ethyl acetate=3:1) to obtain I-96 (11 g, 60% purity).

Preparation of Intermediate 97 (I-97)

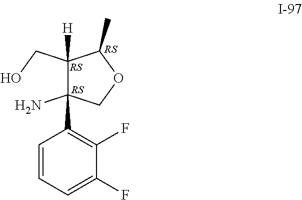

To I-96 (10 g, 41.45 mmol) were added acetic acid (100 mL), then Zn (18.98 g, 290.17 mmol), and the reaction mixture was stirred for overnight at RT. The reaction was filtered over dicalite® and washed with acetic acid and concentrated under reduced pressure. The residue was dissolved in DCM and basified by careful addition of aq. NH₃, the org layer was separated, dried (MgSO₄), filtered and the solvent evaporated under reduced pressure. The residue was purified by flash column chromatography (silica gel, NP, flash purification system; petroleum ether/ethyl acetate=15:1 to petroleum ether/ethyl acetate=1:2). The product fractions were collected and the solvent was evaporated to yield I-97 (6.8 g, 67%).

Preparation of Intermediate 98 (I-98)

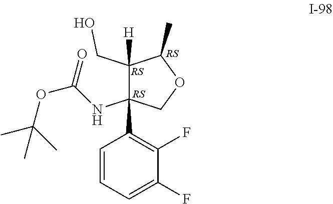

To a stirred solution of I-97 (3 g, 12.33 mmol) in THF (60 mL) were added Et₃N (2.57 mL, 18.49 mmol) and Boc₂O (3.5 g, 16.03 mmol) and the reaction mixture was stirred for 72 h. The reaction mixture was concentrated under reduced pressure. The residue was purified by flash column chromatography (n-heptane/EtOAc 100/0 to 60/40). The product fractions were collected and the solvent was removed in vacuo to yield I-98 (3.1 g, 73%).

Preparation of Intermediate 99 (I-99)

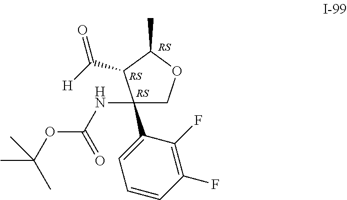 [SMILES: C[C@H]1OC[C@@](NC(=O)OC(C)(C)C)(c2cccc(F)c2F)[C@@H]1C=O]

Dess-Martin periodinane (4.98 g, 11.74 mmol) was added portion-wise over 5 min to a solution of I-98 (3.1 g, 9.03 mmol) in DCM (125 mL) at RT. The mixture was stirred at RT overnight. The mixture was treated with sat. sol. Na₂S₂O₃ (75 mL) and sat. sol. NaHCO₃ (75 mL), stirred for 30 min and extracted with DCM. The org layer was separated, dried (MgSO₄), filtered and concentrated in vacuo. The residue was purified by flash column chromatography (heptane/EtOAc 100/0 to 70/30). The desired fractions were collected and concentrated in vacuo to yield I-99 (1.65 g, 53%).

Preparation of Intermediate 100 (I-100)

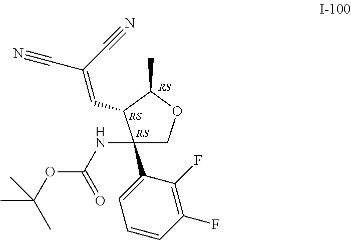 [SMILES: C[C@H]1OC[C@@](NC(=O)OC(C)(C)C)(c2cccc(F)c2F)[C@@H]1C=C(C#N)C#N]

To a stirred solution of I-99 (1.65 g, 4.83 mmol) in MeOH (60 mL) were added MgO (292.23 mg, 7.23 mmol) and malononitrile (478.99 mg, 7.25 mmol), and the reaction mixture was stirred for 20 h at RT. The reaction mixture was then filtered over dicalite® and the solvent was evaporated under reduced pressure to yield I-100, which was used without further purification.

Preparation of Intermediate 101 (I-101)

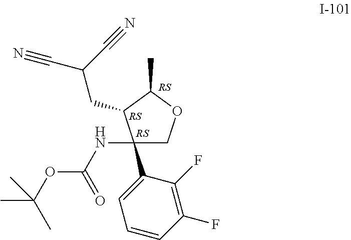 [SMILES: C[C@H]1OC[C@@](NC(=O)OC(C)(C)C)(c2cccc(F)c2F)[C@@H]1CC(C#N)C#N]

To a stirred solution of I-100 (1.88 g, 4.83 mmol) in THF (60 mL) at 0° C., was added NaBH₄ (274.1 mg, 7.25 mmol), and the reaction mixture was stirred for 60 min at 0° C. The reaction mixture was then diluted with H₂O (30 ml) and acidified by addition of HCl (1N). The reaction mixture was diluted further with EtOAc. The org layer was separated, extracted with EtOAc, the combined organic layers were dried (MgSO₄), filtered and the solvent was evaporated under reduced pressure. The reaction mixture was purified with column chromatography (silica gel; n-heptane/EtOAc 100/0 to 60/40). The product fractions were collected and the solvent was evaporated under reduced pressure to yield I-101 (1.5 g, 79%).

Preparation of Intermediate 102 (I-102)

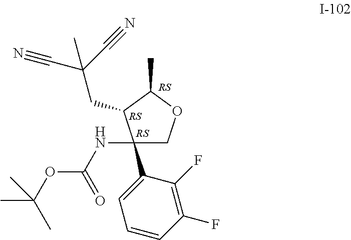 [SMILES: C[C@H]1OC[C@@](NC(=O)OC(C)(C)C)(c2cccc(F)c2F)[C@@H]1CC(C)(C#N)C#N]

To a stirred solution of I-101 (1.5 g, 3.83 mmol) in dry THF (40 mL) under N₂ atmosphere at 0° C. was added NaH (60% dispersion in mineral oil, 199.26 mg, 4.98 mmol), and the mixture was stirred for 15 min at 0° C. under N₂ atmosphere. CH₃I (357.86 μL, 5.75 mmol) was added, the mixture was further stirred for 1 h at 0° C., then H₂O (30 ml) followed by EtOAc (50 ml) were added. The organic layer was separated, dried (MgSO₄), filtered off and the solvent was evaporated under reduced pressure to yield I-102, which was used without further purification.

Preparation of Intermediates 103a (I-103a) and 103b (I-103b)

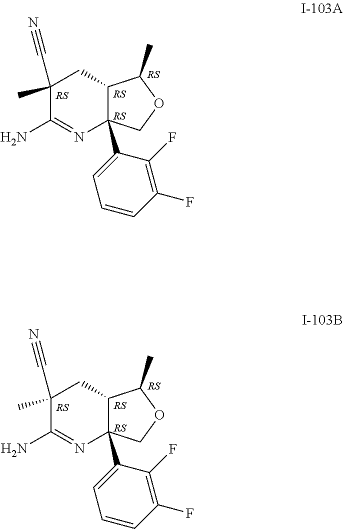 [SMILES: C[C@H]1OC[C@]2(c3cccc(F)c3F)N=C(N)[C@](C)(C#N)C[C@H]12 ; C[C@H]1OC[C@]2(c3cccc(F)c3F)N=C(N)[C@@](C)(C#N)C[C@H]12]

I-102 (1.55 g, 3.83 mmol) was dissolved in formic acid (20 mL) and stirred for 2 h at RT. The solvent was removed in vacuo, the residue was dissolved in DCM (30 ml) and washed with NaHCO₃ sol. (15 ml), the org layer was separated, and the aqueous layer was extracted with DCM, the combined organic layers were dried (MgSO₄), filtered and the solvent was removed in vacuo. The reaction mixture was purified with flash column chromatography (DCM/MeOH 100/0 to 96/4). The product fractions were collected and the solvent was removed in vacuo to yield I-103a (375 mg, 32%) and I-103b (370 mg, 32%).

Preparation of Intermediate 104a (I-104a)

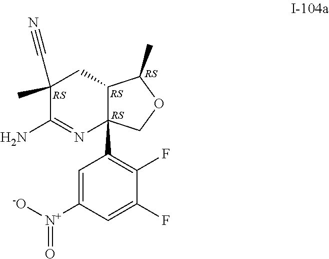

To a stirred solution of I-103a (375 mg, 1.23 mmol) in TFA (8 mL) was added H₂SO₄ (0.66 mL, 12.28 mmol) and the reaction mixture was then cooled down to 0° C. Nitric acid (112.16 μL, 2.46 mmol) was then added in portions over 2 hours and the reaction mixture was stirred at 0° C., until the reaction went to completion. The reaction mixture was poured portionwise into ice/saturated Na₂CO₃ solution (30 ml)/EtOAc (30 ml) mixture. The organic layer was separated and the aqueous layer was extracted twice with EtOAc. The combined organic layers were dried (MgSO₄), filtered and the solvent was removed in vacuo. The crude was purified by flash column chromatography (DCM/NH₃ in MeOH (7N) 100/0 to 98/2). The product fractions were collected and concentrated in vacuo to yield I-104a (395 mg, 92%).

The following intermediates were prepared in an analogous manner from the indicated starting material

| STARTING | |
|---|---|
| MATERIAL | INTERMEDIATE |
| I-103b | 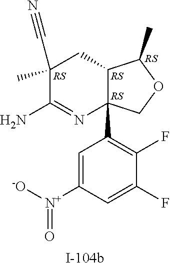 |

Preparation of Intermediate 105a (I-105a)

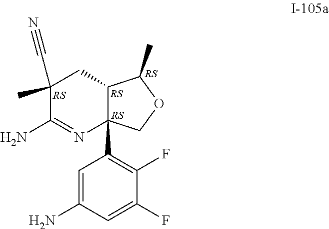 [SMILES: C[C@H]1OC[C@]2(c3cc(N)cc(F)c3F)N=C(N)[C@](C)(C#N)C[C@H]12]

To a solution of I-104a (395 mg, 1.128 mmol) in MeOH (6.5 mL) and water (1.5 mL), were added iron (503.75 mg, 9.02 mmol) and NH₄Cl (603.13 mg, 11.28 mmol) and the reaction mixture was stirred at 70° C. for 1 h. The reaction mixture was then cooled to room temperature, MeOH and DCM were added, and the mixture was filtered over dicalite®, the dicalite® was washed with DCM. The filtrate was then concentrated under reduced pressure and the residue was dissolved in DCM/H₂O mixture. A few drops of a saturated NaHCO3 solution were added. The organic layer was separated and the aqueous layer was extracted with DCM, the combined organic layers were dried (MgSO₄), filtered and concentrated in vacuo. The crude was purified by flash column chromatography (DCM/NH₃ in MeOH (7N) 100/0 to 94/6). The product fractions were collected and concentrated in vacuo to yield I-105a (230 mg, 64%).

The following intermediates were prepared in an analogous manner from the indicated starting material

| STARTING | |
|---|---|
| MATERIAL | INTERMEDIATE |
| I-104b | 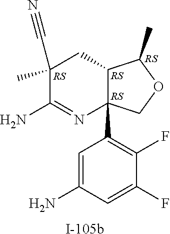 |

B. Preparation of the Final Compounds

### EXAMPLE 1

### Co. No. 1a and 1b

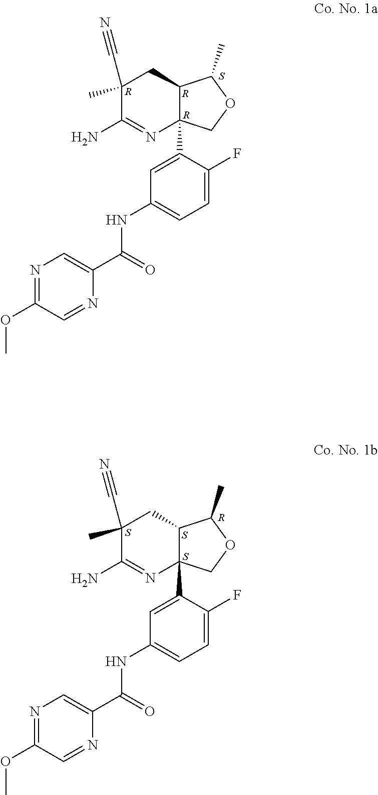

To a stirred solution of I-36 (185 mg, 0.6 mmol) in MeOH (10 mL) at RT, was added HCl (6M in iPrOH, 153.0 μL, 0.9 mmol) and the mixture was stirred for 5 min. Then, 5-methoxypyrazine-2-carboxylic acid (103.7 mg, 0.7 mmol) was added and 5 min later, EDCI.HCl (152.5 mg, 0.8 mmol) was added. The reaction was finished in 10 min. The solvent was removed by evaporation, the residue was dissolved in DCM and washed with aq. sol. Na₂CO₃. The org layer was dried (MgSO₄), filtered and evaporated. The residue was purified by flash chromatography (DCM/MeOH(NH₃(7N)) 100/0 to 90/10). The pure product fractions were collected, concentrated and dried in a vacuum oven at 50° C. to yield a racemic mixture of compounds 1a and b.

25 mg of this racemic mixture was kept aside and the rest was purified via Prep SFC (stationary phase: Chiralpak Diacel AD 20×250 mm, mobile phase: CO₂, EtOH with 0.4% iPrNH₂). The two separate enantiomers a and b were collected, the solvent was evaporated, and the solids were suspended from DIPE, and dried under vacuum at 50° C. yielding Co. No. 1a (65 mg, 24%) and Co. No. 1b (67 mg, 25%).

### EXAMPLE 2

### Co. No. 2a-2f

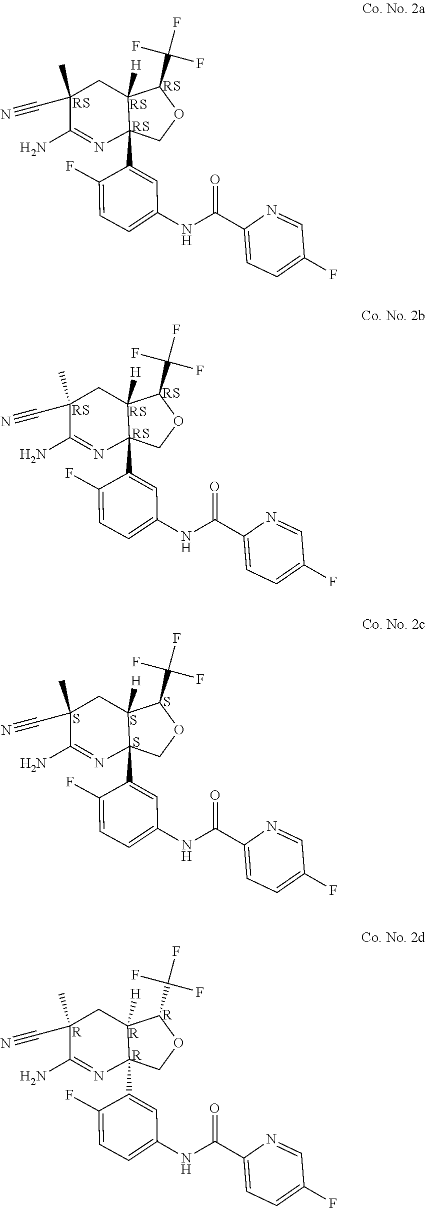

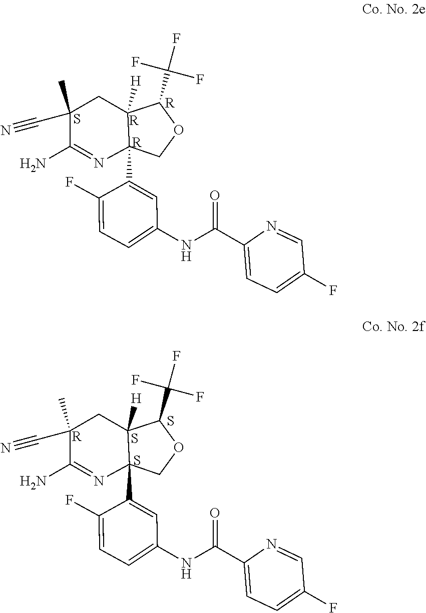

To a stirred solution of I-17 (270 mg, 0.8 mmol) in MeOH (12.1 mL) at RT, was added HCl (6 M in iPrOH, 189.435 μL, 1.137 mmol) and the mixture was stirred for 5 min. Then 5-fluoropicolinic acid (128.5 mg, 0.9 mmol) was added and 5 min later, EDCI.HCl (188.8 mg, 1.0 mmol) was added. The reaction was finished in 10 min. The solvent was removed by evaporation and the residue was taken up in DCM and washed with aq. sol. Na₂CO₃. The organic layer was dried (MgSO₄), filtered and evaporated and the residue was purified by flash chromatography (DCM/methanol(NH₃(7N)) 100/0 to 90/10). The 2 different pure diastereomers of the product were collected and evaporated and purified by Prep SFC:

Purification of diastereomer 1 (Co. No. 2a): (Stationary phase: Chiralcel Diacel OD 20×250 mm, mobile phase: CO₂, EtOH+0.4 iPrNH₂) the product fractions were evaporated, dried under N₂ flow at 50° C. yielding Co. No. 2c (64 mg, 18%) and Co. No. 2d (61 mg, 17%).

Purification of diastereomer 2 (Co. No. 2b): (Stationary phase: Chiralcel Diacel OD 20×250 mm; mobile phase: CO₂, iPrOH+0.4 iPrNH₂) the product fractions were evaporated, coevaporated with DIPE, dried under N₂ flow at 50° C. yielding Co. No. 2e (26 mg, 7%) and Co. No. 2f (24 mg, 7%).

### EXAMPLE 3

### Co. No. 3a-2f

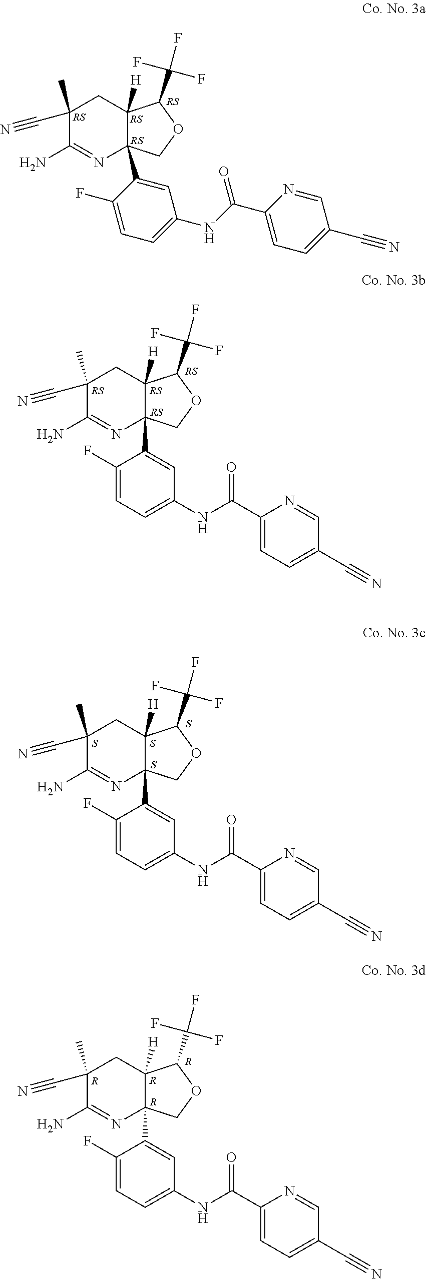

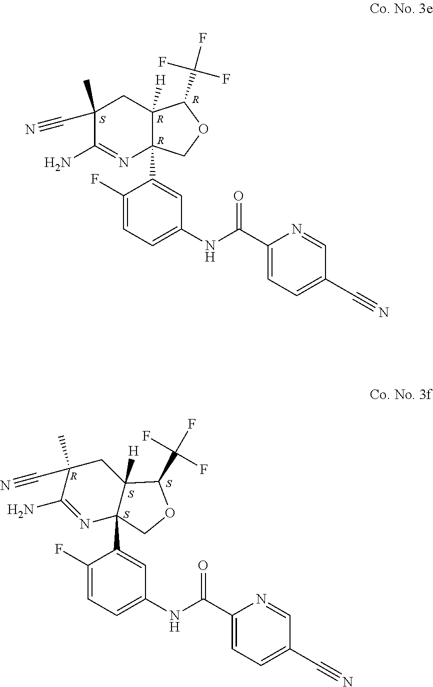

To a stirred solution of I-17 (250 mg, 0.7 mmol) in MeOH (11.2 mL) at RT, was added HCl (6M in iPrOH, 175.4 μL, 1.1 mmol) was added and the mixture was stirred for 5 min. Then 5-cyanopyridine-2-carboxylic acid (118.95 mg, 0.8 mmol) was added and 5 min later, EDCI.HCl (174.9 mg, 0.9 mmol) was added. The reaction was finished in 10 min. The solvent was removed by evaporation and the residue was taken up in DCM and washed with aq. sol. Na₂CO₃. The organic layer was dried (MgSO₄), filtered and the solvent evaporated and the residue was purified by flash chromatography (DCM/MeOH(NH₃(7N)) 100/0 to 90/10). The 2 different pure diastereomers of the product were collected, the solvent was evaporated and coevaporated with DIPE.

These 2 diastereomers (Co. No. 3a and Co. No. 3b) were purified by Prep SFC together with the 2 diastereomers isolated from another reaction performed on 140 mg of 1-17.

Purification of diastereomer 1 (Co. No. 3a): (Stationary phase: Chiralcel Diacel OD 20×250 mm; mobile phase: CO₂, EtOH+0.4 iPrNH₂) followed by drying at 50° C. under N₂ flow for two days, yielding Co. No. 3c (110 mg, 21%) and Co. No. 3d (105 mg, 20%).

Purification of diastereomer 2 (Co. No. 3b): (Stationary phase: Chiralcel Diacel OD 20×250 mm; mobile phase: CO₂, iPrOH+0.4 iPrNH₂) followed by drying at 50° C. under N₂ flow for two days, yielding Co. No. 3e (35 mg, 7%) and Co. No. 3f (37 mg, 7%).

The following compounds were also prepared in an analogous manner:

| STARTING | | |
|---|---|---|
| MATERIAL | REAGENT | COMPOUND |
| I-36 | 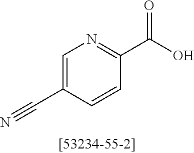 [SMILES: N#Cc1ccc(C(=O)O)nc1] | 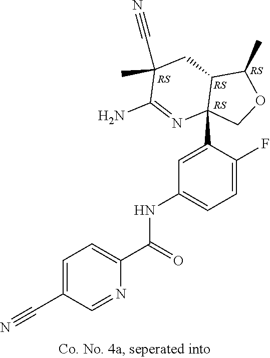 [SMILES: C[C@H]1OC[C@]2(c3cc(NC(=O)c4ccc(C#N)cn4)ccc3F)N=C(N)[C@](C)(C#N)C[C@H]12] |
| | | 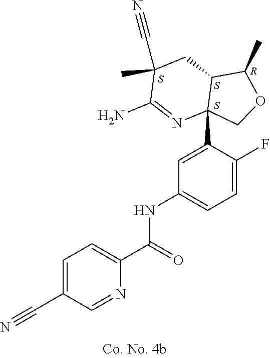 |
| | | 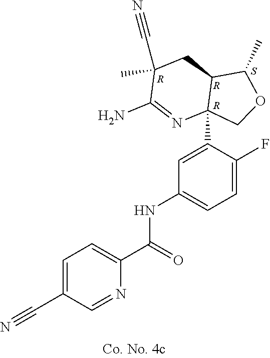 |
| I-38 | 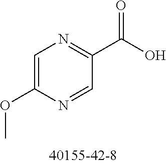 | 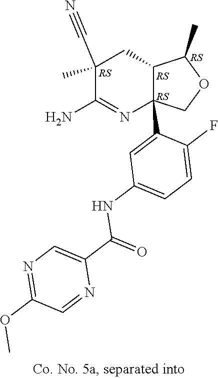 [SMILES: COc1cnc(C(=O)Nc2ccc(F)c([C@]34CO[C@H](C)[C@H]3C[C@](C)(C#N)C(N)=N4)c2)cn1] |
| | | 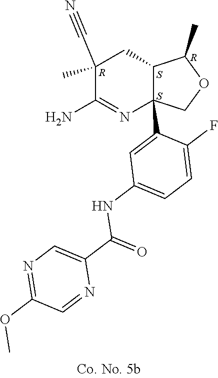 |
| | | 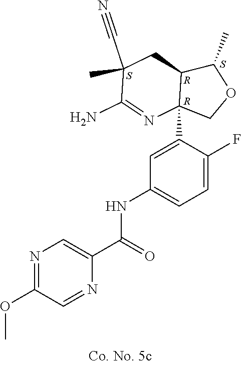 |
| I-38 | 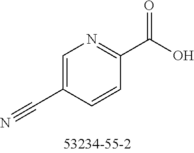 [SMILES: N#Cc1ccc(C(=O)O)nc1] | 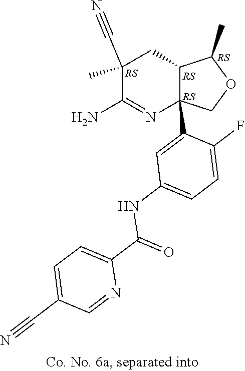 [SMILES: C[C@H]1OC[C@]2(c3cc(NC(=O)c4ccc(C#N)cn4)ccc3F)N=C(N)[C@@](C)(C#N)C[C@H]12] |
| | | 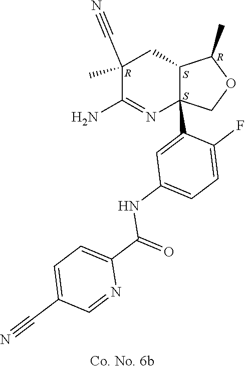 |
| | | 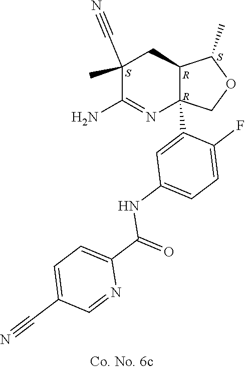 |
| I-17 | 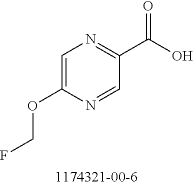 [SMILES: O=C(O)c1cnc(OCF)cn1] | 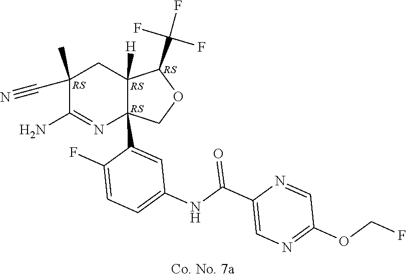 [SMILES: C[C@@]1(C#N)C[C@@H]2[C@@H](C(F)(F)F)OC[C@]2(c2cc(NC(=O)c3cnc(OCF)cn3)ccc2F)N=C1N] |
| | | 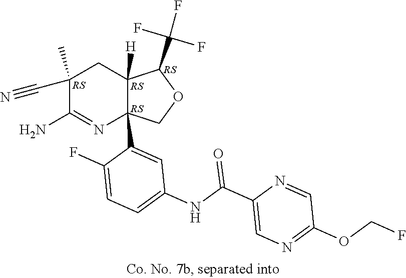 [SMILES: C[C@]1(C#N)C[C@@H]2[C@@H](C(F)(F)F)OC[C@]2(c2cc(NC(=O)c3cnc(OCF)cn3)ccc2F)N=C1N] |
| | | 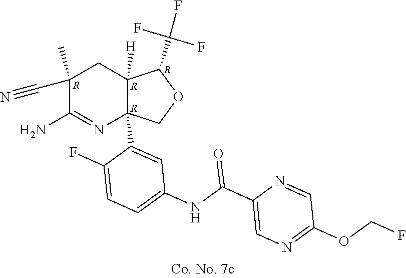 |
| | | 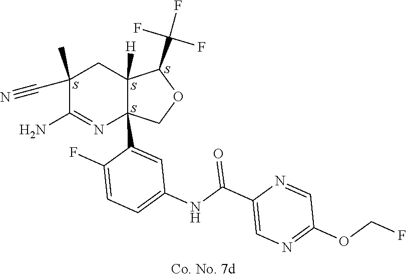 |
| | | 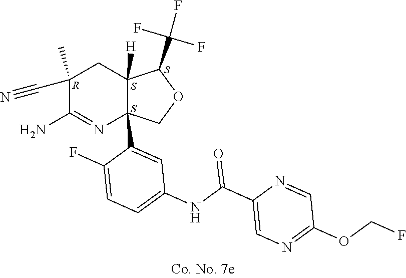 |
| | | 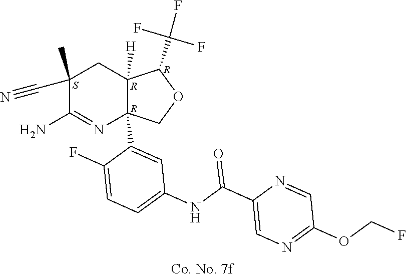 |
| I-17 | 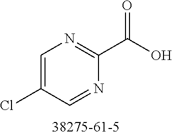 | 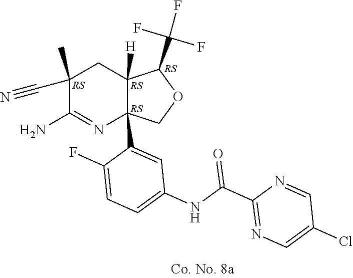 |
| | | 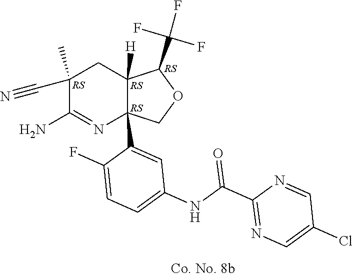 |
| | | 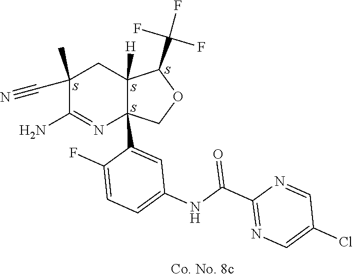 |
| | | 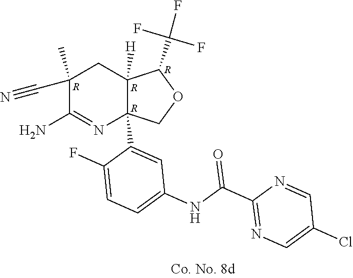 |
| I-36 | 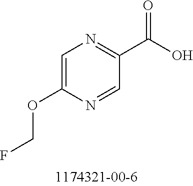 [SMILES: O=C(O)c1cnc(OCF)cn1] | 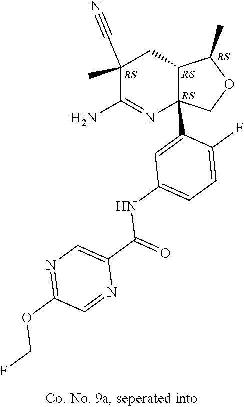 [SMILES: C[C@H]1OC[C@]2(c3cc(NC(=O)c4cnc(OCF)cn4)ccc3F)N=C(N)[C@](C)(C#N)C[C@H]12] |
| | | 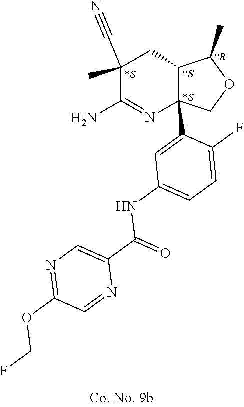 [SMILES: C[C@H]1OC[C@]2(c3cc(NC(=O)c4cnc(OCF)cn4)ccc3F)N=C(N)[C@](C)(C#N)C[C@H]12] |
| | | 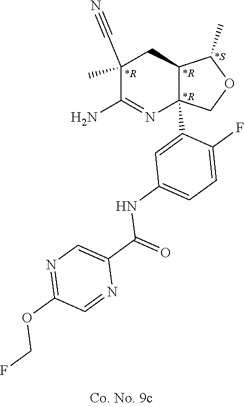 [SMILES: C[C@@H]1OC[C@@]2(c3cc(NC(=O)c4cnc(OCF)cn4)ccc3F)N=C(N)[C@@](C)(C#N)C[C@@H]12] |
| I-36 | 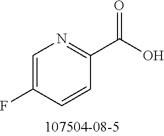 | 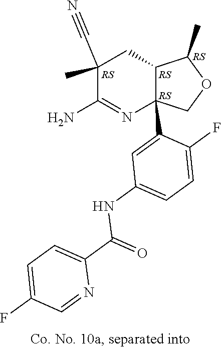 [SMILES: C[C@H]1OC[C@]2(c3cc(NC(=O)c4ccc(F)cn4)ccc3F)N=C(N)[C@](C)(C#N)C[C@H]12] |
| | | 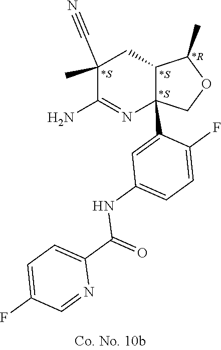 [SMILES: C[C@H]1OC[C@]2(c3cc(NC(=O)c4ccc(F)cn4)ccc3F)N=C(N)[C@](C)(C#N)C[C@H]12] |
| | | 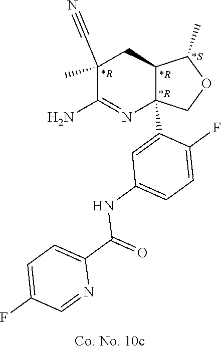 [SMILES: C[C@@H]1OC[C@@]2(c3cc(NC(=O)c4ccc(F)cn4)ccc3F)N=C(N)[C@@](C)(C#N)C[C@@H]12] |

### EXAMPLE 4

### Co. No. 11a and 11b

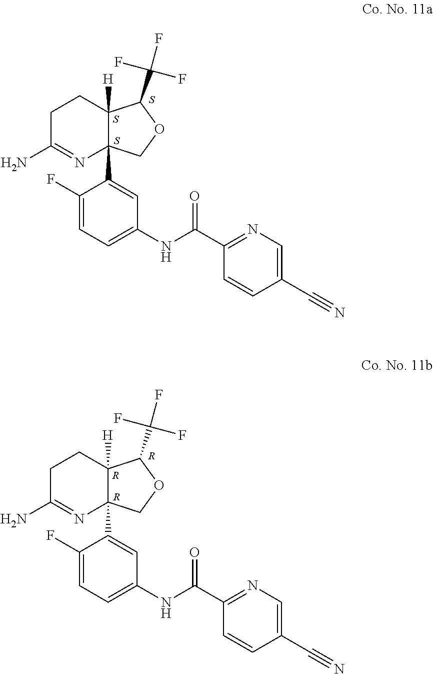

To a stirred solution of 5-cyanopyridine-2-carboxylic acid (1.2 mg, 1.2 mmol) in MeOH (28 mL) was added DMTMM (362.3 mg, 1.2 mmol) and the mixture was stirred at RT. After stirring for 5 min, a solution of I-37 (300 mg, 1.0 mmol) in MeOH (10 mL) was added to the reaction mixture at 0° C. The reaction mixture was stirred at RT for 6 h, then DCM and NaHCO₃ sat. sol. were added. The org layer was separated, dried (MgSO₄), filtered and evaporated. The residue was purified by flash column chromatography (silica, NP, Biotage flash purification system; DCM/NH₃ in MeOH (7N) 100/0 to 90/10). The product fractions were collected and the solvent was evaporated. The residue was suspended from DIPE, the precipitate was filtered off and dried under vacuum to yield 205 mg of a racemic mixture of Co. No. 11a and b, of which 180 mg were purified by Prep SFC (Chiralpak Daicel IC 20×250 mm; mobile phase: CO₂, iPrOH with 0.2% iPrNH₂), the pure fractions were evaporated and suspended from DIPE, the precipitate was filtered off and dried under vacuum at 50° C. yielding Co. No. 11a (70 mg, 17%) and Co. No. 11b (70 mg, 17%).

The following compounds were also prepared in an analogous manner:

| STARTING | | |
|---|---|---|
| MATERIAL | REAGENT | COMPOUND |
| I-37 | 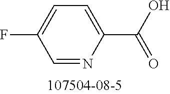 | 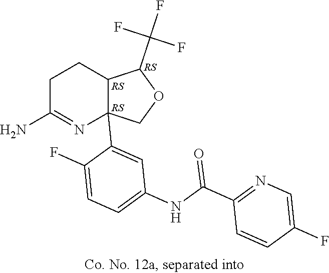 [SMILES: NC1=NC2(c3cc(NC(=O)c4ccc(F)cn4)ccc3F)COC(C(F)(F)F)C2CC1] |
| | | 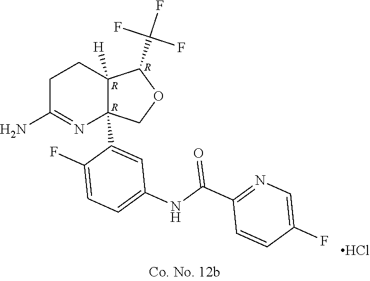 |
| | | 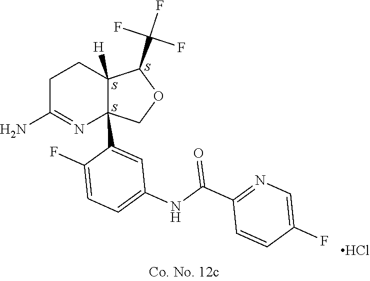 |

### EXAMPLE 5

### Co. No. 13a, 13b, 13c and 13d

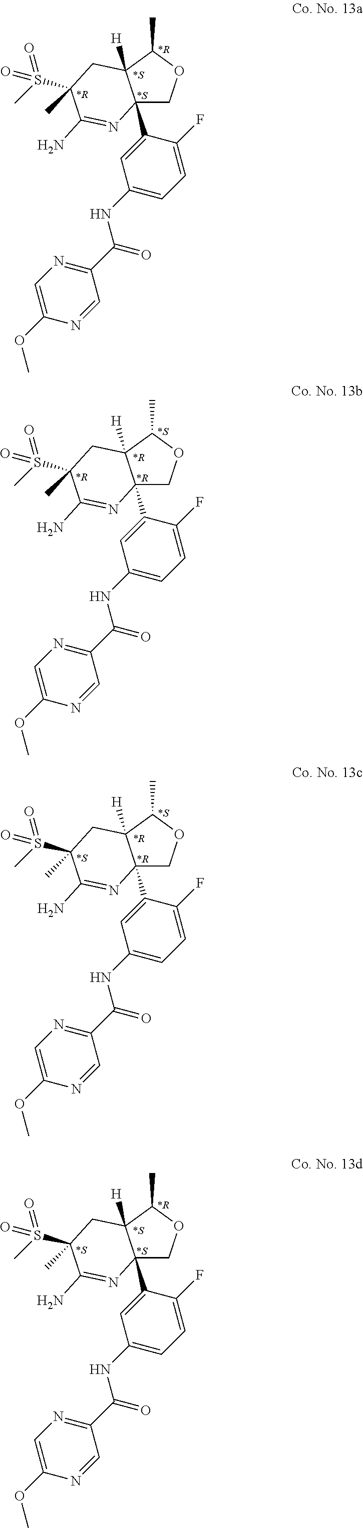 [SMILES: COc1cnc(C(=O)Nc2ccc(F)c([C@]34CO[C@H](C)[C@H]3C[C@@](C)(S(C)(=O)=O)C(N)=N4)c2)cn1 ; COc1cnc(C(=O)Nc2ccc(F)c([C@@]34CO[C@@H](C)[C@@H]3C[C@@](C)(S(C)(=O)=O)C(N)=N4)c2)cn1 ; COc1cnc(C(=O)Nc2ccc(F)c([C@@]34CO[C@@H](C)[C@@H]3C[C@](C)(S(C)(=O)=O)C(N)=N4)c2)cn1 ; COc1cnc(C(=O)Nc2ccc(F)c([C@]34CO[C@H](C)[C@H]3C[C@](C)(S(C)(=O)=O)C(N)=N4)c2)cn1]

A MW tube was loaded with I-39(153 mg, 0.4 mmol), 5-methoxy-2-pyrazinecarboxamide ([19222-85-6], 67.1 mg, 0.4 mmol), CuI (76.4 mg, 0.4 mmol) and K₃PO₄ (154.9 mg, 0.7 mmol) in 1,4-dioxane (3.80 mL, 44.5 mmol). The vial was degassed by bubbling N₂ for a few minutes, then, trans-N,N′-dimethylcyclohexane-1,2-diamine (62.3 mg, 0.4 mmol) was added and, after stirring for 2 min at RT, the mixture was heated for 16 h at 100° C. The mixture was poured into NH₃ in MeOH (7 N) and stirred for 1 h. Next, water and DCM were added and the org layer was separated. The aq. layer was extracted twice with DCM. The organic layer was separated, dried (MgSO₄), filtered and concentrated in vacuo to afford a crude that was purified by flash column chromatography (silica gel; DCM/7 N NH₃ in MeOH 100/0 to 95/5). The product fractions were evaporated to afford a fraction, containing 4 diastereomers.

A purification was performed via Prep SFC (stationary phase: Chiralcel Diacel OD 20×250 mm; mobile phase: CO₂, EtOH+0.4 iPrNH₂) yielding 4 fractions:

Fraction a was triturated in DIPE and dried (vacuum oven, 50° C.). DIPE could not be removed by drying the sample in a vacuum oven or re-dissolving and triturating it again in DIPE, so the compound was then redissolved in MeOH/DCM, solvents were concentrated and the compound was dried (vacuum oven, 50° C., on) to afford Co. No. 13a (27 mg, 15%)

Fraction b contained 9% impurity and was purified by Prep HPLC (stationary phase: RP XBridge Prep C18 ODB-5 μm, 30×250 mm; mobile phase: 0.25% NH₄HCO₃ solution in water, CH₃CN) yielding Co. No. 13b (13.8 mg, 8%)

Fraction c contained 13% impurity and was purified by Prep HPLC (stationary phase: RP XBridge Prep C18 ODB-5 μm, 30×250 mm; mobile phase: 0.25% NH₄HCO₃ solution in water, CH₃CN) yielding Co. No. 13c (18.3 mg, 10%)

Fraction d was triturated in DIPE and dried (vacuum oven, 50° C., on) and repurified by flash column chromatography (silica gel; eluent: DCM/7 N NH₃ in MeOH, 100:0 to 95:5) to afford Co. No. 13d (11.6 mg, 7%)

### EXAMPLE 6

### Co. No. 14

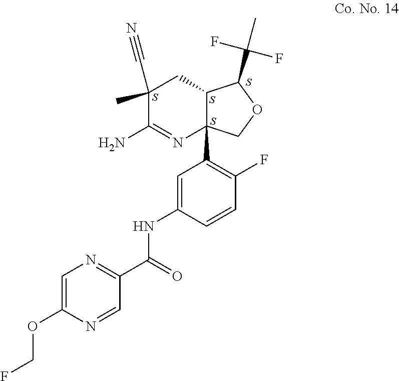

To a stirred solution of I-60 (30 mg, 0.085 mmol) in MeOH (5 mL) at RT, was added HCl (6M in 2-propanol, 21.29 μL, 0.13 mmol) and the mixture was stirred for 5 min. Then 5-(fluoromethoxy)-2-pyrazinecarboxylic acid ([1174321-00-6], 14.65 mg, 0.085 mmol) was added and 5 min later, EDCI (21.22 mg, 0.11 mmol) was added. The reaction was finished in 10 min. The solvent was removed under vacuo, the residue was taken up in DCM and washed with aq. sol. Na₂CO₃. The organic layer was dried (MgSO₄), filtered and evaporated. The residue was purified by flash chromatography (DCM:methanol, 100:0 to 95:5). The product fractions were collected and evaporated, then dried for 48 h under vacuum at 50° C. to yield compound 14 (10 mg, 23%).

The relative stereochemistry was confirmed by NMR.

### EXAMPLE 7

### Co. No. 15

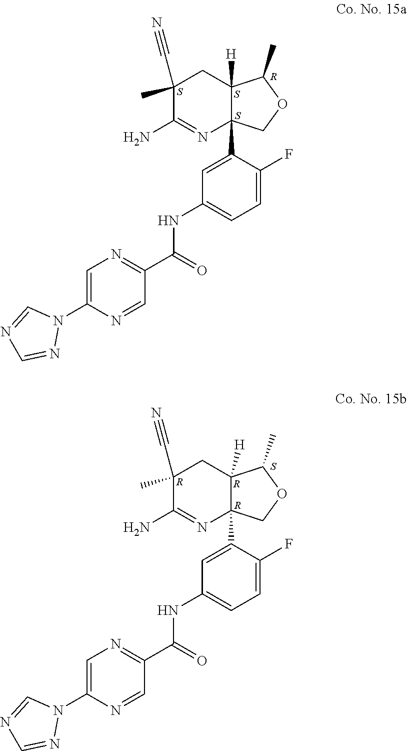

To a stirred solution of I-36 (75 mg, 0.25 mmol) in MeOH (3.55 mL) was added HCl (5M in 2-propanol, 59.53 μL, 0.30 mmol) at RT. Then 5-(1H-1,2,4-triazol-1-yl)-2-pyrazinecarboxylic acid ([1200497-38-6], 65.5 mg, 0.34 mmol) and EDCI (61.82 mg, 0.32 mmol) were added and the mixture was stirred at RT for 60 min. The reaction mixture was concentrated and the residue was taken up in DCM and washed with Na₂CO₃ sat. sol. The organic layer was separated, and concentrated under reduced pressure and the residue was purified by column chromatography (silica gel; eluent from 100% DCM up to 10% MeOH_NH₃ (7N) in DCM). Product fractions were collected and the solvent was removed under reduced pressure to afford the desired compound as a solid (50 mg) which was purified by Prep SFC (stationary phase: Chiralcel Diacel OJ 20×250 mm; mobile phase: CO₂, iPrOH+0.4 iPrNH₂). The desired fractions were collected and evaporated and the precipitate was suspended from DIPE and dried under N₂ flow at 50° C. yielding Co. No. 15a (13 mg, 11%) and Co. No. 15b (13 mg, 11%).

### EXAMPLE 8

### Co. No. 16a

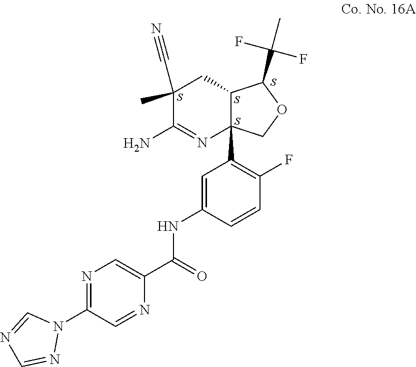

To a stirred solution of I-60 (100 mg, 0.284 mmol) in MeOH (17 mL) at RT, was added HCl (6M in 2-propanol, 70.95 0.426 mmol) and the mixture was stirred for 5 min. Then 5-(1H-1,2,4-triazol-1-yl)-2-pyrazinecarboxylic acid ([1200497-38-6], 65.10 mg, 0.341 mmol).was added and 5 min later, EDCI (70.73 mg, 0.369 mmol) was added. The reaction was finished in 20 min. The solvent was removed under vacuo, the residue was taken up in DCM and washed with aq. sol. Na₂CO₃. The organic layer was dried (MgSO₄), filtered and evaporated. The residue was purified by flash chromatography (DCM:MeOH, 100:0 to 96:4). The product fractions were collected and evaporated, then dried for 48 h under vacuum at 50° C. to yield compound 16a (43 mg, 29%).

### EXAMPLE 9

### Co. No. 21a

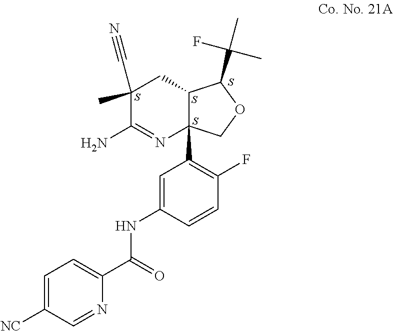

To a stirred solution of I-72a (80 mg, 0.23 mmol) in MeOH (13 mL) at RT, was added HCl (6M in 2-propanol, 57.41 μL, 0.344 mmol) and the mixture was stirred for 5 min. Then 5-cyanopyridine-2-carboxylic acid ([53234-55-2], 40.8 mg. 0.276 mmol) was added and 5 min later, EDCI (57.22 mg, 0.299 mmol) was added. The reaction was finished in 30 min. The solvent was removed under vacuo, the residue was dissolved in DCM and washed with aq. sol. Na₂CO₃. The organic layer was dried (MgSO₄), filtered and evaporated. The residue was purified by flash chromatography (DCM : methanol, 100:0 to 96:4). The product fractions were collected and evaporated. The residue was purified further via Prep HPLC (Stationary phase: RP XBridge Prep C18 OBD-10μm, 30×150 mm; mobile phase: 0.25% NH₄HCO₃ solution in water, CH3CN)

The product fractions were collected and the solvent was evaporated under reduced pressure. The crude product was triturated with DIPE, filtered off and then dried under vacuum at 50° C. to yield compound 21a (20 mg, 15%).

Compound 21b was prepared in an analogous manner starting from I-72b.

### EXAMPLE 10

### Co. No.22a

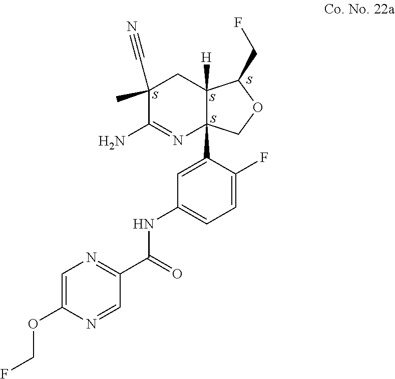

To a stirred solution of I-83a (82 mg, 0.256 mmol) in MeOH (5 mL) at RT, was added HCl (6M in 2-propanol, 64 μ0.33 mmol) and the mixture was stirred for 5 min. Then 5-(fluoromethoxy)-2-pyrazinecarboxylic acid ([1174321-00-6], 52.9 mg, 0.307 mmol) was added and 5 min later, EDCI (63.79 mg, 0.333 mmol) was added. The reaction was finished in 20 min. The solvent was removed under vacuo, the residue was dissolved in DCM and washed with aq. sol. Na₂CO₃. The organic layer was dried (MgSO₄), filtered and evaporated. The residue was purified by flash chromatography (DCM:MeOH, 100:0 to 96:4). The desired product fractions were collected and evaporated and the precipitate was titurated with DIPE and dried under vacuum at 60° C. yielding Co. No. 22a (46 mg, 37%).

Compound 24b was prepared in an analogous manner starting from I-83b

### EXAMPLE 11

### Co. No.25

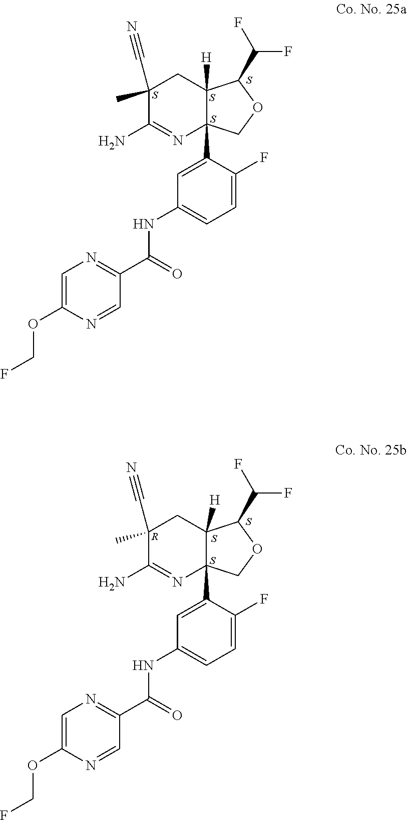

To a stirred solution of I-94 (90 mg, 0.266 mmol) in MeOH (5 mL) at RT, was added HCl (6M in 2-propanol, 53.2 μL, 0.32 mmol) and the mixture was stirred for 5 min. Then 5-(fluoromethoxy)-2-pyrazinecarboxylic acid ([1174321-00-6], 54.94 mg, 0.319 mmol) was added and 5 min later, EDCI (66.29 mg, 0.346 mmol) was added. The reaction was finished in 60 min. The solvent was removed under vacuo, the residue was dissolved in DCM and washed with aq. sol. Na₂CO₃. The organic layer was dried (MgSO₄), filtered and evaporated. The residue was purified by flash chromatography (DCM:NH₃ in methanol (7N), 100:0 to 90:10) to give the 2 pure diastereomers. The desired product fractions were collected and evaporated and the precipitate was suspended from DIPE and dried under vacuum at 60° C. for 36 hours yielding Co. No. 25a (44 mg, 34%) and Co. No. 25b (22 mg, 17%).

### EXAMPLE 12

### Co. No. 31

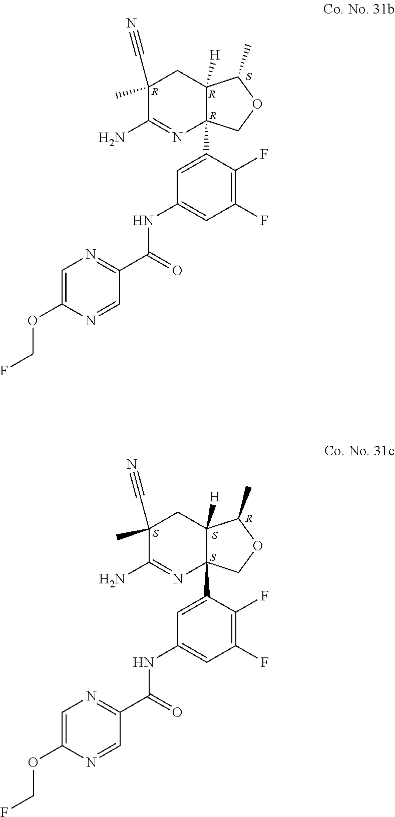

To a stirred solution of I-105a (30 mg, 0.094 mmol) in MeOH (1.76 mL) was added HCl (6M in 2-propanol, 23.41 μL, 0.14 mmol) at RT. Then 5-(fluoromethoxy)-2-pyrazinecarboxylic acid ([1174321-00-6], (20.15 mg, 0.117 mmol) and EDCI (26.93 mg, 0.14 mmol) were added and the mixture was stirred at RT for 60 min. The reaction mixture was concentrated and the residue was taken up in DCM and washed with Na₂CO₃ sat. sol. The organic layer was separated, and concentrated under reduced pressure and the residue was purified by column chromatography (silica gel; eluent from 100% DCM up to 3% MeOH_NH₃ (7N) in DCM). Product fractions were collected and the solvent was removed under reduced pressure to afford the desired compound as a solid (40 mg) which was purified by Prep SFC (Stationary phase: Chiralpak Diacel AD 20×250 mm; mobile phase: CO₂, EtOH+0.4 iPrNH₂). The desired fractions of both compounds were collected and evaporated with CH₃CN and dried in the vacuum oven at 60° C. yielding Co. No. 31b (17 mg, 38%) and Co. No. 31c (18 mg, 40%).

Compound 31a and compound 31d were prepared in an analogous manner starting from I-105b.

Table 1 lists the compounds that were prepared or that can be prepared by analogy to one of the above Examples. In case no salt form is indicated, the compound was obtained as a free base. ‘Ex. No.’ refers to the Example number according to which protocol the compound was synthesized. ‘Co. No.’ means compound number.

TABLE 1

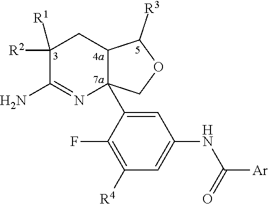

| Co. No. | Ex. No. | R¹ | R² | R³ | R⁴ | Ar | Stereochemistry/Salt |
|---|---|---|---|---|---|---|---|
| 1a 1b | 1 | CH₃ | CN | CH₃ | H | 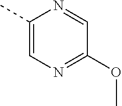 | 3R,4aR,5S,7aR 3S,4aS,5R,7aS |
| 2c 2d 2e 2f | 2 | CH₃ | CN | CF₃ | H | 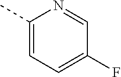 | 3S,4aS,5S,7aS 3R,4aR,5R,7aR 3S,4aR,5R,7aR 3R,4a5,5S,7aS |
| 3c 3d 3e 3f | 3 | CH₃ | CN | CF₃ | H | 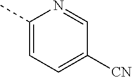 | 3S,4aS,5S,7aS 3R,4aR,5R,7aR 3S,4aR,5R,7aR 3R,4aS,5S,7aS |
| 4b 4c | 3 | CH₃ | CN | CH₃ | H | 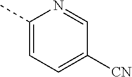 | 3S,4aS,5R,7aS 3R,4aR,5S,7aR |
| 5b 5c | 3 | CH₃ | CN | CH₃ | H | 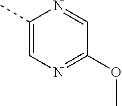 | 3R,4aS,5R,7aS 3S,4aR,5S,7aR |
| 6b 6c | 3 | CH₃ | CN | CH₃ | H | 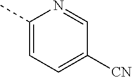 | 3R,4aS,5R,7aS 3S,4aR,5S,7aR |
| 7c 7d 7e 7f | 3 | CH₃ | CN | CF₃ | H | 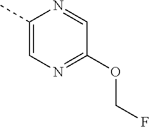 | 3R,4aR5R,7aR 3S,4aS,5S,7aS 3R,4aS,5S,7aS 3S,4aR,5R,7aR |
| 8c 8d | 3 | CH₃ | CN | CF₃ | H | 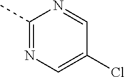 | 3S,4aS,5S,7aS 3R,4aR,5R,7aR |
| 9b 9c | 3 | CH₃ | CN | CH₃ | H | 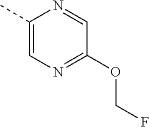 | 3*S,4a*S,5*R,7a*S 3*R,4a*R,5*S,7a*R |
| 10b 10c | 3 | CH₃ | CN | CH₃ | H | 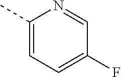 | 3*S,4a*S,5*R,7a*S 3*R,4a*R,5*S,7a*R |
| 11a 11b | 4 | H | H | CF₃ | H | 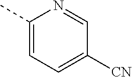 | 4aS,5S,7aS 4aR,5R,7aR |
| 12b 12c | 4 | H | H | CF₃ | H | 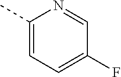 | 4aR,5R,7aR •HCl 4aS,5S,7aS •HCl |
| 13a 13b 13c 13d | 5 | CH₃ | MeSO₂ | CH₃ | H | 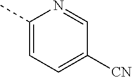 | 3*R,4a*S,5*R,7a*S 3*R,4a*R,5*S,7a*R 3*S,4a*R,5*S,7a*R 3*S,4a*S,5*R,7a*S |
| 14 | 6 | CH₃ | CN | CF₂CH₃ | H | 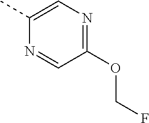 | 3S,4aS,5S,7aS |
| 15a | 7 | CH₃ | CN | CH₃ | H | 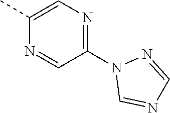 | 3S,4aS,5R,7aS |
| 15b | 7 | CH₃ | CN | CH₃ | H | 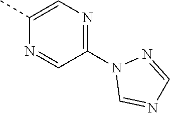 | 3R,4aR,5S,7aR |
| 16 | 8 | CH₃ | CN | CF₂CH₃ | H | 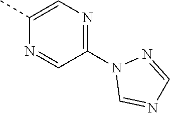 | 3RS,4aS,5S,7aS |
| 16a | 8 | CH₃ | CN | CF₂CH₃ | H | 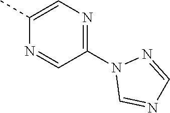 | 3S,4aS,5S,7aS |
| 17 | 6 | CH₃ | CN | CF₂CH₃ | H | 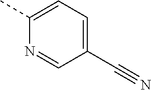 | 3RS,4aS,5S,7aS |
| 17a | 6 | CH₃ | CN | CF₂CH₃ | H | 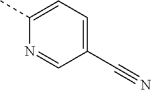 | 3S,4aS,5S,7aS |
| 18 | 6 | CH₃ | CN | CF₂CH₃ | H | 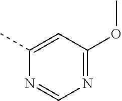 | 3RS,4aS,5S,7aS |
| 18a | 6 | CH₃ | CN | CF₂CH₃ | H | 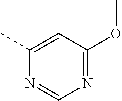 | 3S,4aS,5S,7aS |
| 19 | 10 | CH₃ | CN | C(CH₃)₂F | H | 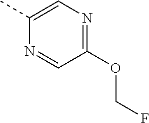 | 3RS,4aS,5S,7aS |
| 19a | 9 | CH₃ | CN | C(CH₃)₂F | H | 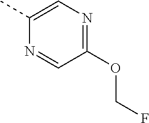 | 3S,4aS,5S,7aS |
| 20 | 10 | CH₃ | CN | C(CH₃)₂F | H | 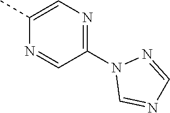 | 3RS,4aS,5S,7aS |
| 21 | 9 | CH₃ | CN | C(CH₃)₂F | H | 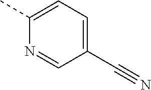 | 3RS,4aS,5S,7aS |
| 21a | 9 | CH₃ | CN | C(CH₃)₂F | H | 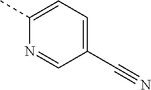 | 3S,4aS,5S,7aS |
| 21b | 9 | CH₃ | CN | C(CH₃)₂F | H | 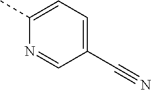 | 3R,4aS,5S,7aS |
| 22 | 10 | CH₃ | CN | CH₂F | H | 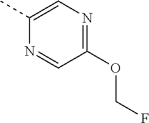 | 3RS,4aS,5S,7aS |
| 22a | 10 | CH₃ | CN | CH₂F | H | 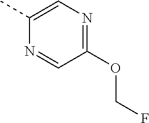 | 3S,4aS,5S,7aS |
| 23 | 10 | CH₃ | CN | CH₂F | H | 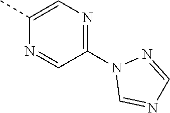 | 3RS,4aS,5S,7aS |
| 23a | 10 | CH₃ | CN | CH₂F | H | 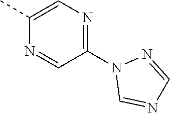 | 3S,4aS,5S,7aS |
| 24 | 10 | CH₃ | CN | CH₂F | H | 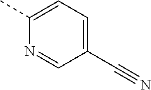 | 3RS,4aS,5S,7aS |
| 24a | 10 | CH₃ | CN | CH₂F | H | 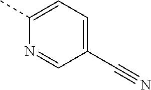 | 3S,4aS,5S,7aS |
| 24b | 10 | CH₃ | CN | CH₂F | H | 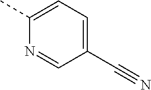 | 3R,4aS,5S,7aS |
| 25 | 11 | CH₃ | CN | CHF₂ | H | 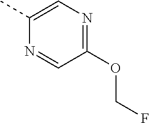 | 3RS,4aS,5S,7aS |
| 25a | 11 | CH₃ | CN | CHF₂ | H | 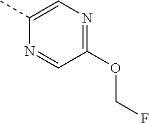 | 3S,4aS,5S,7aS |
| 25b | 11 | CH₃ | CN | CHF₂ | H | 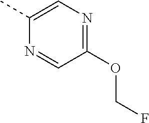 | 3R,4aS,5S,7aS |
| 26 | 11 | CH₃ | CN | CHF₂ | H | 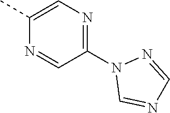 | 3RS,4aS,5S,7aS |
| 26a | 11 | CH₃ | CN | CHF₂ | H | 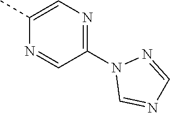 | 3S,4aS,5S,7aS |
| 26b | 11 | CH₃ | CN | CHF₂ | H | 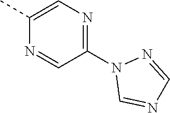 | 3R,4aS,5S,7aS |
| 27 | 11 | CH₃ | CN | CHF₂ | H | 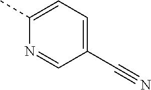 | 3RS,4aS,5S,7aS |
| 27a | 11 | CH₃ | CN | CHF₂ | H | 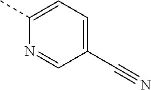 | 3S,4aS,5S,7aS |
| 28 | 10 | CH₃ | CN | 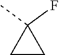 | H | 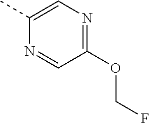 | 3RS,4aS,5S,7aS |
| 29 | 10 | CH₃ | CN | 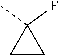 | H | 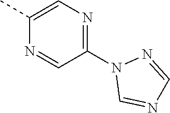 | 3RS,4aS,5S,7aS |
| 30 | 10 | CH₃ | CN | 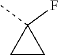 | H | 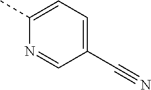 | 3RS,4aS,5S,7aS |
| 31a | 12 | CH₃ | CN | CH₃ | F | 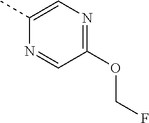 | 3R,4aS,5R,7aS |
| 31b | 12 | CH₃ | CN | CH₃ | F | 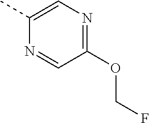 | 3R,4aR,5S,7aR |
| 31c | 12 | CH₃ | CN | CH₃ | F | 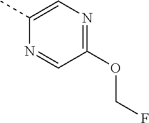 | 3S,4aS,5R,7aS |
| 31d | 12 | CH₃ | CN | CH₃ | F | 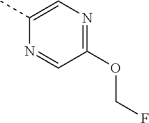 | 3S,4aR,5S,7aR |
| 32a | 12 | CH₃ | CN | CH₃ | F | 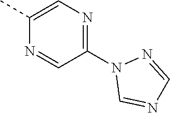 | 3S,4aS,5R,7aS |
| 32b | 12 | CH₃ | CN | CH₃ | F | 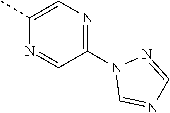 | 3R,4aR,5S,7aR |
| 33a | 12 | CH₃ | CN | CH₃ | F | 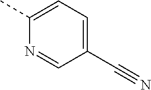 | 3S,4aR,5S,7aR |
| 33b | 12 | CH₃ | CN | CH₃ | F | 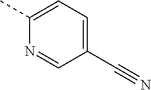 | 3S,4aS,5R,7aS |
| 33c | 12 | CH₃ | CN | CH₃ | F | 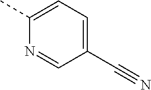 | 3R,4aS,5R,7aS |
| 33d | 12 | CH₃ | CN | CH₃ | F | 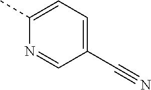 | 3R,4aR,5S,7aR |
| 34a | 3 | CH₃ | CN | CF₃ | H | 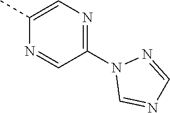 | 3*R,4a*R,5*R,7a*R |
| 34b | 3 | CH₃ | CN | CF₃ | H | 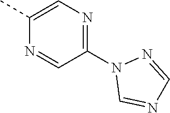 | 3*S,4a*S,5*S,7a*S |
| 35 | 1 | CH₃ | CN | CH₃ | H | 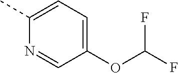 | 3RS,4aRS,5RS,7aRS |
| 35a | 1 | CH₃ | CN | CH₃ | H | 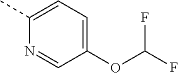 | 3*S,4a*S,5*R,7a*S |
| 35b | 1 | CH₃ | CN | CH₃ | H | 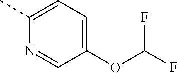 | 3*R,4a*R,5*S,7a*R |
| 36a | 1 | CH₃ | CN | CH₃ | H | 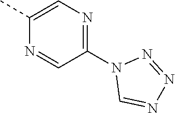 | 3S,4aS,5R,7aS |
| 36b | 1 | CH₃ | CN | CH₃ | H | 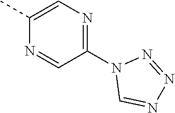 | 3R,4aR,5S,7aR |
| 37 | 6 | CH₃ | CN | CF₂CH₃ | H | 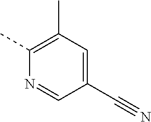 | 3S,4aS,5S,7aS |
| 38 | 6 | CH₃ | CN | CF₂CH₃ | H | 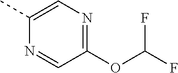 | 3S,4aS,5S,7aS |

C. Analytical Part

LC-MS (Liquid Chromatography/Mass Spectrometry)

LCMS General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software. Compounds are described by their experimental retention times (Rₜ) and ions. If not specified otherwise in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc.). For molecules with multiple isotopic patterns (Br, Cl.), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica., “Q-Tof” Quadrupole Time-of-flight mass spectrometers, “CLND”, ChemiLuminescent Nitrogen Detector, “ELSD” Evaporative Light Scanning Detector.

TABLE 2
a. LCMS Method codes (Flow expressed in mL/min; column
temperature (T) in ° C.; Run time in minutes)

| | | | | | Flow | Run |
|---|---|---|---|---|---|---|
| Method | | | | | ------ | time |
| code | Instrument | Column | Mobile phase | Gradient | Col T | (min) |
| 1 | Waters: | Waters: | A: 10 mM | From | 0.7 | 3.5 |
| | Acquity ® | HSS T3 | CH₃COONH₄ | 100% A to | ------- | |
| | UPLC ®- | (1.8 μm, | in 95% H₂O + | 5% A in | 55 | |
| | DAD and | 2.1*100 | 5% CH₃CN | 2.10 min, | | |
| | SQD | mm) | B: CH₃CN | to 0% A in | | |
| | | | | 0.90 min, | | |
| | | | | to 5% A in | | |
| | | | | 0.5 min | | |
| 2 | Waters: | Waters: | A: 10 mM | From 95% | 0.8 | 2 |
| | Acquity ® | BEH C18 | CH₃COONH₄ | A to 5% A | ------- | |
| | UPLC ®- | (1.7 μm, | in 95% H₂O + | in 1.3 min, | 55 | |
| | DAD and | 2.1*50 | 5% CH₃CN | held for | | |
| | SQD | mm) | B: CH₃CN | 0.7 min. | | |
| 3 | Agilent | YMC- | A: 0.1% | From 95% | 2.6 | 6.2 |
| | 1100 | pack | HCOOH in | A to 5% A | ------- | |
| | HPLC | ODS-AQ | H₂O | in 4.8 min, | 35 | |
| | DAD | C18 (50 × | B: CH₃CN | held for | | |
| | LC/MS | 4.6 mm, 3 | | 1.0 min, to | | |
| | G1956A | μm) | | 95% A in | | |
| | | | | 0.2 min. | | |
| 4 | Agilent | YMC- | A: 0.1% | From 95% | 2.6 | 6.8 |
| | 1260 | pack | HCOOH in | A to 5% A | ------- | |
| | Infinity | ODS-AQ | H₂O | in 4.8 min, | 35 | |
| | DAD | C18 (50 × | B: CH₃CN | held for | | |
| | TOF- | 4.6 mm, 3 | | 1.0 min, to | | |
| | LC/MS | μm) | | 95% A in | | |
| | G6224A | | | 0.2 min. | | |

b. LCMS data for compounds

| Co. | | | | LCMS |
|---|---|---|---|---|
| No. | Rₜ (min) | [M + H]⁺ | [M − H]⁻ | Method |
| 1a + b | 1.67 | 439.4 | 437.3 | 1 |
| 1a | 1.67 | 439 | 437 | 1 |
| 1b | 1.67 | 439 | 437 | 1 |
| 2a | 1.92 | 480.1 | 478.1 | 1 |
| 2c | 1.91 | 480 | 478 | 1 |
| 2d | 1.91 | 480 | 478 | 1 |
| 2e | 1.79 | 480 | 478 | 1 |
| 2f | 1.79 | 480 | 478 | 1 |
| 3a | 1.88 | 487.1 | 485.1 | 1 |
| 3b | 1.75 | 487.3 | 485.1 | 1 |
| 3c | 1.88 | 487 | 485 | 1 |
| 3d | 1.88 | 487 | 485 | 1 |
| 3e | 1.77 | 487 | 485 | 1 |
| 3f | 1.77 | 487 | 485 | 1 |
| 4a | 0.82 | 433.2 | 431.2 | 2 |
| 4b | 1.63 | 433 | 431 | 1 |
| 4c | 1.63 | 433 | 431 | 1 |
| 5a | 1.56 | 439.2 | 437.1 | 1 |
| 5b | 1.56 | 439 | 437 | 1 |
| 5c | 1.56 | 439 | 437 | 1 |
| 6a | 1.53 | 433.3 | 431.1 | 1 |
| 6b | 1.53 | 433 | 431 | 1 |
| 6c | 1.53 | 433 | 431 | 1 |
| 7a | 1.89 | 511.3 | 509.2 | 1 |
| 7c | 1.9 | 511 | 509 | 1 |
| 7d | 1.9 | 511 | 509 | 1 |
| 7e | 1.79 | 511 | 509 | 1 |
| 7f | 1.79 | 511 | 509 | 1 |
| 8a | 1.75 | 497.1 | 495 | 1 |
| 8c | 1.78 | 497 | 495 | 1 |
| 8d | 1.77 | 497 | 495 | 1 |
| 9b | 1.68 | 457 | 455 | 1 |
| 9c | 1.68 | 457 | 455 | 1 |
| 10b | 1.7 | 426 | 424 | 1 |
| 10c | 1.7 | 426 | 424 | 1 |
| 11a + b | 0.82 | 448.1 | 446.1 | 2 |
| 11a | 0.85 | 448 | 446 | 2 |
| 11b | 0.84 | 448 | 446 | 2 |
| 12a | 0.86 | 441.1 | 439.1 | 2 |
| 12b | 0.89 | 441.2 | 438.9 | 2 |
| 12c | 0.89 | 441 | 439 | 2 |
| 13a | 1.54 | 492 | 490 | 1 |
| 13b | 1.53 | 492 | 490 | 1 |
| 13c | 1.55 | 492 | 490 | 1 |
| 13d | 1.55 | 492 | 490 | 1 |
| 14 | 1.01 | 507 | 505 | 2 |
| 15a | 1.53 | 476 | 474 | 1 |
| 15b | 1.53 | 476 | 474 | 1 |
| 16a | 1.70 | 526 | 524 | 1 |
| 17a | 1.83 | 483 | 481 | 1 |
| 18a | 0.98 | 489 | 487 | 2 |
| 19a | 1.83 | 503 | 501 | 1 |
| 21a | 0.94 | 479 | 477 | 2 |
| 21b | 2.35 | 479 | 477 | 3 |
| 22a | 1.14 | 475 | 473 | 3 |
| 23a | 1.99 | 494 | 492 | 3 |
| 24a | 2.03 | 451 | 449 | 3 |
| 24b | 1.6 | 451 | 449 | 1 |
| 25a | 0.85 | 493 | 491 | 2 |
| 25b | 0.91 | 493 | 491 | 2 |
| 26a | 1.52 | 512 | 510 | 1 |
| 27a | 0.89 | 469 | 467 | 2 |
| 31a | 1.68 | 475 | 473 | 1 |
| 31b | 1.77 | 475 | 473 | 1 |
| 31c | 1.77 | 475 | 473 | 1 |
| 31d | 1.68 | 475 | 473 | 1 |
| 32a | 1.64 | 494 | 492 | 1 |
| 32b | 1.64 | 494 | 492 | 1 |
| 33a | 1.64 | 451 | 449 | 1 |
| 33b | 1.75 | 451 | 449 | 1 |
| 33c | 1.65 | 451 | 449 | 1 |
| 33d | 1.75 | 451 | 449 | 1 |
| 34a | 1.76 | 530 | 529 | 1 |
| 34b | 1.76 | 530 | 529 | 1 |
| 35 | 1.76 | 474 | 472 | 1 |
| 35a | 1.76 | 474 | 472 | 1 |
| 35b | 1.76 | 474 | 472 | 1 |
| 36a | 1.55 | 476 | 474 | 1 |
| 36b | 1.55 | 476 | 474 | 1 |
| 37 | 2.47 | 497 | 495 | 4 |
| 38 | 2.56 | 525 | 523 | 3 |

SFCMS-Methods

General Procedure For SFC-MS Methods

The SFC measurement was performed using an Analytical Supercritical fluid chromatography (SFC) system composed by a binary pump for delivering carbon dioxide (CO2) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 3
Analytical SFC-MS Methods (Flow expressed in mL/min; column
temperature (T) in ° C.; Run time in minutes, Backpressure (BPR) in bars.

| Method | | Mobile | | Flow | Run time |
|---|---|---|---|---|---|
| code | Column | Phase | Gradient | Col T | BPR |
| 1 | Daicel Chiralpak ® | A: CO₂ | 10%-50% B | 2.5 | 9.5 |
| | AD3 column (3.0 | B: EtOH + 0.2% | in 6 min, hold | 40 | 110 |
| | μm, 150 × 4.6 | iPrNH₂ | 3.5 min | | |
| | mm) | | | | |
| 2 | Daicel Chiralpak ® | A: CO₂ | 10%-50% B | 2.5 | 9.5 |
| | AD3 column (3.0 | B: | in 6 min, hold | 40 | 110 |
| | μm, 150 × 4.6 | MeOH + 0.2% | 3.5 min | | |
| | mm) | iPrNH₂ | | | |
| 3 | Daicel Chiralpak ® | A: CO₂ | 30% B hold 4 | 5 | 7 |
| | OD-H column | B: | min, to 50% | 40 | 110 |
| | (5.0 μm, 250 × 4.6 | EtOH + 0.2% | in 1 min hold | | |
| | mm) | iPrNH2 | 2 min | | |
| 4 | Daicel Chiralpak ® | A: CO₂ | 10%-50% B | 2.5 | 9.5 |
| | OD3 column (3.0 | B: EtOH + 0.2% | in 6 min, hold | 40 | 110 |
| | μm, 150 × 4.6 | iPrNH₂ | 3.5 min | | |
| | mm) | | | | |
| 5 | Daicel Chiralpak ® | A: CO₂ | 35% B hold 4 | 5 | 7 |
| | OD-H column | B: | min, to 50% | 40 | 110 |
| | (5.0 μm, 250 × 4.6 | iPrOH + 0.2% | in 1 min hold | | |
| | mm) | iPrNH2 | 2 min | | |
| 6 | Daicel Chiralpak ® | A: CO₂ | 30% B hold 4 | 5 | 7 |
| | OD-H column | B: | min, to 50% | | |
| | (5.0 pm, 250 × 4.6 | iPrOH + 0.2% | in 1 min hold | 40 | 110 |
| | mm) | iPrNH₂ | 2 min | | |
| 7 | Daicel Chiralpak ® | A: CO₂ | 20% B hold 4 | 5 | 7 |
| | OJ-H column (5.0 | B: EtOH + 0.2% | min, to 50% | 40 | 110 |
| | μm, 250 × 4.6 | iPrNH₂ | in 1 min hold | | |
| | mm) | | 2 min | | |
| 8 | Daicel Chiralpak ® | A: CO₂ | 40% B hold 4 | 5 | 7 |
| | AD-H column | B: EtOH + 0.2% | min, to 50% | 40 | 110 |
| | (5.0 μm, 250 × 4.6 | iPrNH₂ | in 1 min hold | | |
| | mm) | | 2 min | | |
| 9 | Daicel Chiralpak ® | A: CO₂ | 35% B hold 4 | 5 | 7 |
| | AD-H column | B: EtOH + 0.2% | min, to 50% | 40 | 110 |
| | (5.0 μm, 250 × 4.6 | iPrNH₂ | in 1 min hold | | |
| | mm) | | 2 min | | |
| 10 | Daicel Chiralpak ® | A: CO₂ | 25% B hold | 3 | 15 |
| | ID-H column (5.0 | B: | 15 min | 40 | 110 |
| | μm, 250 × 4.6 | iPrOH + 0.2% | | | |
| | mm) | iPrNH₂ | | | |
| 11 | Daicel Chiralpak ® | A: CO₂ | 15% B hold | 3 | 15 |
| | ID-H column (5.0 | B: | 15 min | 40 | 110 |
| | μm, 250 × 4.6 | iPrOH + 0.2% | | | |
| | mm) | iPrNH₂ | | | |
| 12 | Daicel Chiralpak ® | A: CO₂ | 40% B hold 4 | 5 | 7 |
| | OD-H column | B: | min, to 50% | 40 | 110 |
| | (5.0 μm, 250 × 4.6 | iPrOH + 0.2% | in 1 min hold | | |
| | mm) | iPrNH₂ | 2 min | | |
| 13 | Daicel Chiralpak ® | A: CO₂ | 10% to 50% B | 2.5 | 9.5 |
| | OJ3 column | B: | in 6 min, hold | 40 | 130 |
| | (3.0 μm, 150 × 4.6 | iPrOH + 0.2% | 3.5 min | | |
| | mm) | iPrNH₂ | | | |
| 14 | Daicel Chiralpak ® | A: CO₂ | 10%-50% B | 2.5 | 9.5 |
| | AD3 column (3.0 | B: EtOH + 0.2% | in 6 min, hold | 40 | 130 |
| | μm, 150 × 4.6 | iPrNH2 | 3.5 min | | |
| | mm) | | | | |
| | Daicel Chiralpak ® | A: CO₂ | 10%-50% B | 2.5 | 9.5 |
| | OJ3 column | B: EtOH + 0.2% | in 6 min, hold | 40 | 130 |
| | (3.0 μm, 150 × 4.6 | iPrNH2 | 3.5 min | | |
| | mm) | | | | |

Analytical SFC data-Rₜ means retention time (in minutes), [M + H]⁺ means
the protonated mass of the compound, method refers to the method used for (SFC)MS
analysis of enantiomerically pure compounds.

| | | | Isomer | |
|---|---|---|---|---|
| Co. | | | Elution | SFCMS |
| No. | Rₜ (min) | [M + H]⁺ | Order | Method |
| 1a | 2.28 | 438 | A | 9 |
| 1b | 3.54 | 439 | B | 9 |
| 2c | 1.07 | 480 | A | 3 |
| 2d | 1.63 | 480 | B | 3 |
| 2e | 1.37 | 480 | A | 6 |
| 2f | 1.78 | 480 | B | 6 |
| 3c | 1.18 | 487 | A | 8 |
| 3d | 2.1 | 487 | B | 8 |
| 3e | 1.44 | 487 | A | 5 |
| 3f | 1.87 | 487 | B | 5 |
| 4b | 1.6 | 433 | A | 7 |
| 4c | 2.47 | 433 | B | 7 |
| 5b | 1.53 | 439 | A | 9 |
| 5c | 2.17 | 439 | B | 9 |
| 6b | 1.74 | 433 | A | 9 |
| 6c | 2.41 | 433 | B | 9 |
| 7c | 3.45 | 511 | A | 1 |
| 7d | 4.87 | 511 | B | 1 |
| 7e | 3.32 | 511 | A | 2 |
| 7f | 5.44 | 511 | B | 2 |
| 8c | 3.97 | 497 | A | 4 |
| 8d | 5.4 | 497 | B | 4 |
| 9b | 1.27 | 457 | A | 12 |
| 9c | 2.5 | 457 | B | 12 |
| 10b | 1.24 | 426 | A | 5 |
| 10c | 1.9 | 425 | B | 5 |
| 11a | 4.26 | 448 | A | 10 |
| 11b | 4.26 | 447 | B | 10 |
| 12b | 6.37 | 441 | A | 11 |
| 12c | 6.52 | 441 | B | 11 |
| 15a | 3.36 | 476 | A | 13 |
| 15b | 4.04 | 476 | B | 13 |
| 31b | 4.43 | 475 | A | 14 |
| 31c | 4.9 | 475 | B | 14 |
| 31a | 4.14 | 475 | A | 14 |
| 31d | 5.08 | 475 | B | 14 |
| 32a | 3.15 | 494 | B | 15 |
| 32b | 2.88 | 494 | A | 15 |
| 33a | 4.85 | 451 | B | 14 |
| 33c | 4.21 | 451 | A | 14 |
| 33b | 2.51 | 451 | A | 15 |
| 33d | 3.04 | 451 | B | 15 |
| 35a | 2.32 | 474 | A | 15 |
| 35b | 2.86 | 474 | B | 15 |
| 36a | 3.99 | 476 | A | 15 |
| 36b | 4.6 | 476 | B | 15 |

Isomer Elution Order:
A means first eluting isomer;
B means second eluting isomer.

NMR

For a number of compounds, ¹H NMR spectra were recorded on a Bruker DPX-400 spectrometer operating at 400 MHz, on a Bruker DPX-360 operating at 360 MHz, or on a Bruker Avance 600 spectrometer operating at 600 MHz, or on a Bruker Ultrashield AV300 MHz operating at 300 MHz, using CHLOROFORM-d (deuterated chloroform, CDCl₃) or DMSO-d₆ (deuterated DMSO, dimethyl-d6 sulfoxide) or BENZENE-d₆ (deuterated benzene, C₆D₆) or ACETONE-d₆ (deuterated acetone, (CD₃)₂CO) as solvents. Chemical shifts (δ) are reported in parts per million (ppm) relative to tetramethylsilane (TMS), which was used as internal standard.

TABLE 4
¹H NMR results

| Co. | |
|---|---|
| No. | ¹H NMR result |
| 1b | ¹H NMR (360 MHz, CHLOROFORM-d) δ ppm 1.35 (d, J = 6.22 Hz, 3 H) 1.67 (s, 3 |
| | H) 1.89 (dd, J = 14.64, 5.12 Hz, 1 H) 2.29 (dd, J = 14.64, 5.49 Hz, 1 H) 2.45 (dt, |
| | J = 8.14, 5.08 Hz, 1 H) 3.91 (dd, J = 8.96, 2.01 Hz, 1 H) 4.06 (s, 3 H) 4.30 (quin, |
| | J = 6.40 Hz, 1 H) 4.58 (dd, J = 8.97, 0.91 Hz, 1 H) 4.80 (br s, 2 H) 7.07 (dd, J = 11.71, |
| | 8.78 Hz, 1 H) 7.58 (dd, J = 7.14, 2.74 Hz, 1 H) 7.74 (ddd, J = 8.78, 4.03, 2.93 Hz, 1 |
| | H) 8.14 (d, J = 1.46 Hz, 1 H) 8.99 (d, J = 1.46 Hz, 1 H) 9.48 (s, 1 H) |
| 3c | ¹H NMR (360 MHz, DMSO-d₆) δ ppm 1.65 (s, 3 H) 1.71 (br dd, J = 15.7, 4.0 Hz, 1 |
| | H) 2.40 (dd, J = 15.6, 2.7 Hz, 1 H) 3.03-3.14 (m, 1 H) 3.95 (dd, J = 8.1, 2.6 Hz, 1 H) |
| | 4.44 (d, J = 8.1 Hz, 1 H) 4.61-4.72 (m, 1 H) 6.63 (br s, 2 H) 7.22 (dd, J = 12.1, 8.8 |
| | Hz, 1 H) 7.79 (dd, J = 7.3, 2.6 Hz, 1 H) 7.91 (dt, J = 8.7, 3.3 Hz, 1 H) 8.28 (d, J = 8.4 |
| | Hz, 1 H) 8.58 (dd, J = 8.1, 1.8 Hz, 1 H) 9.20 (d, J = 2.2 Hz, 1 H) 11.01 (s, 1 H) |
| 2c | ¹H NMR (360 MHz, CHLOROFORM-d) δ ppm 1.70 (s, 3 H) 1.85 (dd, J = 15.4, 4.4 |
| | Hz, 1 H) 2.51 (dd, J = 15.2, 3.1 Hz, 1 H) 3.27-3.36 (m, 1 H) 4.05 (dd, J = 8.4, 2.2 |
| | Hz, 1 H) 4.63 (d, J = 8.4 Hz, 1 H) 4.65-4.73 (m, 1 H) 4.94 (br s, 2 H) 7.11 (dd, |
| | J = 11.5, 8.6 Hz, 1 H) 7.61 (td, J = 8.3, 2.7 Hz, 1 H) 7.64-7.73 (m, 2 H) 8.32 (dd, |
| | J = 8.6, 4.6 Hz, 1 H) 8.45 (d, J = 2.6 Hz, 1 H) 9.79 (s, 1 H) |
| 4b | ¹H NMR (360 MHz, CHLOROFORM-d) δ ppm 1.35 (d, J = 5.85 Hz, 3 H) 1.68 (s, 3 |
| | H) 1.89 (dd, J = 14.64, 5.12 Hz, 1 H) 2.30 (dd, J = 14.64, 5.49 Hz, 1 H) 2.45 (dt, |
| | J = 8.05, 5.31 Hz, 1 H) 3.90 (dd, J = 9.15, 1.83 Hz, 1 H) 4.30 (quin, J = 6.40 Hz, 1 H) |
| | 4.59 (dd, J = 9.15, 0.73 Hz, 1 H) 7.09 (dd, J = 11.71, 8.78 Hz, 1 H) 7.62 (dd, J = 7.14, |
| | 2.74 Hz, 1 H) 7.77 (ddd, J = 8.78, 4.03, 2.93 Hz, 1 H) 8.20 (dd, J = 8.23, 2.01 Hz, 1 |
| | H) 8.42 (dd, J = 8.05, 0.73 Hz, 1 H) 8.89 (dd, J = 2.01, 0.91 Hz, 1 H) 9.83 (s, 1 H) |
| 7d | ¹H NMR (360 MHz, DMSO-d₆) δ ppm 1.65 (s, 3 H) 1.70 (dd, J = 15.6, 3.8 Hz, 1 H) |
| | 2.40 (dd, J = 15.7, 2.9 Hz, 1 H) 3.04-3.12 (m, 1 H) 3.94 (dd, J = 8.1, 2.9 Hz, 1 H) |
| | 4.44 (d, J = 8.1 Hz, 1 H) 4.61-4.71 (m, 1 H) 6.13 (s, 1 H) 6.28 (s, 1 H) 6.63 (br s, 2 |
| | H) 7.21 (dd, J = 11.9, 9.0 Hz, 1 H) 7.79 (dd, J = 7.5, 2.7 Hz, 1 H) 7.84-7.91 (m, 1 H) |
| | 8.58 (d, J = 1.5 Hz, 1 H) 8.96 (d, J = 1.1 Hz, 1 H) 10.79 (s, 1 H) |
| 8c | ¹H NMR (360 MHz, CHLOROFORM-d) δ ppm 1.73 (s, 3 H) 1.84 (dd, J = 15.37, |
| | 4.39 Hz, 1 H) 2.49 (dd, J = 15.19, 3.11 Hz, 1 H) 3.23-3.29 (m, 1 H) 4.05 (dd, |
| | J = 8.23, 2.38 Hz, 1 H) 4.63-4.72 (m, 2 H) 7.11 (dd, J = 11.34, 8.78 Hz, 1 H) 7.43- |
| | 7.55 (m, 1 H) 7.88 (dd, J = 7.32, 2.93 Hz, 1 H) 8.89 (s, 2 H) 9.75 (s, 1 H) |
| 9b | ¹H NMR (360 MHz, DMSO-d₆) δ ppm 1.26 (d, J = 6.22 Hz, 3 H) 1.56-1.69 (m, 4 |
| | H) 2.19-2.34 (m, 2 H) 3.76 (dd, J = 8.23, 2.38 Hz, 1 H) 4.16-4.26 (m, 1 H) 4.39 (d, |
| | J = 9.88 Hz, 1 H) 6.20 (d, J = 51.59 Hz, 2 H) 7.16 (dd, J = 11.71, 8.78 Hz, 1 H) 7.75 |
| | (dd, J = 7.32, 2.56 Hz, 1 H) 7.79-7.85 (m, 1 H) 8.58 (d, J = 1.10 Hz, 1 H) 8.95 (d, |
| | J = 1.46 Hz, 1 H) 10.72 (s, 1 H) |
| 11a | ¹H NMR (360 MHz, DMSO-d₆) δ ppm 1.68-1.77 (m, 2 H) 2.01 (dt, J = 17.38, 4.30 |
| | Hz, 1 H) 2.22-2.35 (m, 1 H) 2.93-3.00 (m, 1 H) 3.89 (dd, J = 7.68, 3.29 Hz, 1 H) |
| | 4.27 (d, J = 8.05 Hz, 1 H) 4.53 (quin, J = 7.41 Hz, 1 H) 5.76 (br s, 2 H) 7.17 (dd, |
| | J = 12.08, 8.78 Hz, 1 H) 7.88 (dt, J = 8.69, 3.34 Hz, 1 H) 7.93 (dd, J = 7.32, 2.56 Hz, 1 |
| | H) 8.28 (d, J = 7.68 Hz, 1 H) 8.58 (dd, J = 8.23, 2.01 Hz, 1 H) 9.20 (d, J = 1.46 Hz, 1 |
| | H) 10.87 (br s, 1 H) |
| 12c | ¹H NMR (600 MHz, DMSO-d₆) δ ppm 1.88-1.95 (m, 1 H) 1.96-2.04 (m, 1 H) |
| | 2.67 (ddd, J = 19.10, 6.20, 3.40 Hz, 1 H) 3.02 (ddd, J = 18.80, 10.40, 7.00 Hz, 1 H) |
| | 3.23 (dt, J = 7.50, 4.10 Hz, 1 H) 4.28 (dd, J = 9.83, 2.35 Hz, 1 H) 4.40 (d, J = 9.83 Hz, |
| | 1 H) 4.89 (quin, J = 7.15 Hz, 1 H) 7.35 (dd, J = 11.88, 8.95 Hz, 1 H) 7.98-8.02 (m, 2 |
| | H) 8.14 (ddd, J = 9.00, 4.30, 2.50 Hz, 1 H) 8.25 (dd, J = 8.73, 4.62 Hz, 1 H) 8.61 (s, 1 |
| | H) 8.75 (d, J = 2.79 Hz, 1 H) 9.41 (s, 1 H) 10.44 (s, 1 H) 10.84 (s, 1 H) |
| 13a | ¹H NMR (360 MHz, CHLOROFORM-d) δ ppm 1.03 (d, J = 6.59 Hz, 3 H) 1.82 (s, 3 |
| | H) 2.21 (d, J = 9.15 Hz, 2 H) 2.38-2.48 (m, 1 H) 3.05 (s, 3 H) 3.79 (d, J = 9.88 Hz, 1 |
| | H) 4.07 (s, 3 H) 4.08-4.12 (m, 1 H) 4.67 (d, J = 9.88 Hz, 1 H) 7.08 (dd, J = 11.71, |
| | 8.78 Hz, 1 H) 7.66 (dd, J = 7.32, 2.56 Hz, 1 H) 7.94 (ddd, J = 8.78, 4.21, 2.74 Hz, 1 |
| | H) 8.17 (d, J = 1.46 Hz, 1 H) 9.02 (d, J = 1.46 Hz, 1 H) 9.53 (s, 1 H) |
| 14 | ¹H NMR (400 MHz, CHLOROFORM-d) δ ppm 1.69 (s, 3 H) 1.71-1.87 (m, 4 H) |
| | 2.53 (dd, J = 14.9, 3.6 Hz, 1 H) 3.29-3.34 (m, 1 H) 3.98 (br d, J = 8.5 Hz, 1 H) 4.43 |
| | (ddd, J = 17.6, 8.5, 3.4 Hz, 1 H) 4.56 (d, J = 8.5 Hz, 1 H) 4.86 (br s, 2 H) 6.16 (dq, |
| | J = 51.0, 2.0 Hz, 2 H) 7.10 (dd, J = 11.7, 8.9 Hz, 1 H) 7.63 (dd, J = 6.9, 2.8 Hz, 1 H) |
| | 7.65-7.71 (m, 1 H) 8.28 (d, J = 1.2 Hz, 1 H) 9.07 (d, J = 1.6 Hz, 1 H) 9.46 (s, 1 H) |
| 15a | ¹H NMR (400 MHz, CHLOROFORM-d) δ ppm 1.36 (d, J = 6.1 Hz, 3 H) 1.71 (s, 3 |
| | H) 1.92 (dd, J = 14.6, 5.3 Hz, 1 H) 2.32 (dd, J = 14.6, 5.7 Hz, 1 H) 2.46 (dt, J = 8.1, 5.3 |
| | Hz, 1 H) 3.92 (dd, J = 9.2, 1.8 Hz, 1 H) 4.31 (quin, J = 6.4 Hz, 1 H) 4.60 (dd, J = 9.0, |
| | 0.8 Hz, 1 H) 4.84 (s, 2 H) 7.11 (dd, J = 11.6, 8.7 Hz, 1 H) 7.64 (dd, J = 7.1, 2.6 Hz, 1 |
| | H) 7.72-7.80 (m, 1 H) 8.23 (s, 1 H) 9.24-9.26 (m, 2 H) 9.33 (d, J = 1.2 Hz, 1 H) |
| | 9.54 (s, 1 H) |
| 16a | ¹H NMR (300 MHz, CHLOROFORM-d) δ ppm 1.52-1.89 (m, 7 H) 2.46 (br dd, |
| | J = 15.05, 2.54 Hz, 1 H) 3.20-3.29 (m, 1 H) 3.92 (br d, J = 8.11 Hz, 1 H) 4.25-4.43 |
| | (m, 1 H) 4.49 (br d, J = 8.39 Hz, 1 H) 7.04 (br t, J = 10.24 Hz, 1 H) 7.53-7.70 (m, 2 |
| | H) 8.15 (s, 1 H) 9.16 (br s, 1 H) 9.17 (br s, 1 H) 9.24 (br s, 1 H) 9.49 (br s, 1 H) |
| 17a | ¹H NMR (400 MHz, CHLOROFORM-d) δ ppm 1.69 (s, 3 H) 1.71-1.87 (m, 4 H) |
| | 2.53 (dd, J = 15.2, 3.5 Hz, 1 H) 3.31 (dt, J = 8.4, 4.0 Hz, 1 H) 3.98 (br d, J = 8.4 Hz, 1 |
| | H) 4.42 (ddd, J = 17.7, 8.5, 3.1 Hz, 1 H) 4.56 (d, J = 8.4 Hz, 1 H) 4.86 (br s,2 H) 7.11 |
| | (dd, J = 11.6, 8.7 Hz, 1 H) 7.64-7.73 (m, 2 H) 8.21 (dd, J = 8.1, 2.0 Hz, 1 H) 8.43 |
| | (dd, J = 8.1, 0.9 Hz, 1 H) 8.90 (dd, J = 2.0, 0.9 Hz, 1 H) 9.81 (br s, 1 H) |
| 18a | ¹H NMR (400 MHz, CHLOROFORM-d) δ ppm 1.68 (s, 3 H) 1.69-1.85 (m, 4 H) |
| | 2.52 (dd, J = 15.3, 3.5 Hz, 1 H) 3.26-3.33 (m, 1 H) 3.98 (br d, J = 8.5 Hz, 1 H) 4.07 |
| | (s, 3 H) 4.43 (ddd, J = 17.6, 8.9, 3.3 Hz, 1 H) 4.56 (d, J = 8.1 Hz, 1 H) 4.86 (br s, 2 H) |
| | 7.10 (dd, J = 11.6, 8.7 Hz, 1 H) 7.58 (d, J = 0.8 Hz, 1 H) 7.63-7.73 (m, 2 H) 8.81 (d, |
| | J = 1.2 Hz, 1 H) 9.84 (s, 1 H) |
| 19a | ¹H NMR (400 MHz, CHLOROFORM-d) δ ppm 1.49 (dd, J = 21.77, 4.27 Hz, 6 H) |
| | 1.71 (s, 3 H) 1.85 (dd, J = 15.05, 4.48 Hz, 1 H) 2.48 (dd, J = 14.85, 3.87 Hz, 1 H) |
| | 3.10-3.17 (m, 1 H) 3.95 (br d, J = 8.14 Hz, 1 H) 4.28 (dd, J = 12.61, 8.54 Hz, 1 H) 4.52 |
| | (br d, J = 8.54 Hz, 1 H) 6.06-6.23 (m, 2 H) 7.09 (dd, J = 11.60, 8.75 Hz, 1 H) 7.60 |
| | (dd, J = 7.12, 2.64 Hz, 1 H) 7.68-7.74(m, 1 H) 8.29 (d, J = 1.22 Hz, 1 H) 9.07 (d, |
| | J = 1.22 Hz, 1 H) 9.47 (br s, 1 H) |
| 21a | ¹H NMR (400 MHz, DMSO-d₆) δ ppm 1.36 (s, 3 H) 1.42 (s, 3 H) 1.59-1.66 (m, 4 |
| | H) 2.34 (dd, J = 15.42, 2.98 Hz, 1 H) 2.84 (dt, J = 8.98, 3.57 Hz, 1 H) 3.79 (dd, |
| | J = 7.73, 2.98 Hz, 1 H) 4.23 (dd, J = 15.46, 8.93 Hz, 1 H) 4.31 (d, J = 7.99 Hz, 1 H) |
| | 6.41 (s, 2 H) 7.18 (dd, J = 11.98, 8.80 Hz, 1 H) 7.76 (dd, J = 7.35, 2.64 Hz, 1 H) 7.85 |
| | (ddd, J = 8.85, 4.17, 2.67 Hz, 1 H) 8.28 (dd, J = 8.17, 0.89 Hz, 1 H) 8.56 (dd, J = 8.17, |
| | 2.06 Hz, 1 H) 9.19 (dd, J = 2.13, 0.87 Hz, 1 H) 10.94(s, 1 H) |
| 22a | ¹H NMR (300 MHz, CHLOROFORM-d) δ ppm 1.71 (s, 3 H) 1.93 (br dd, J = 14.85, |
| | 4.54 Hz, 1 H) 2.40 (br dd, J = 14.85, 5.09 Hz, 1 H) 2.88-2.99 (m, 1 H) 3.97 (br d, |
| | J = 8.66 Hz, 1 H) 4.35-4.68 (m, 4 H) 6.16 (br d, J = 51.14 Hz, 2 H) 7.10 (dd, |
| | J = 11.48, 8.87 Hz, 1 H) 7.63 (dd, J = 6.94, 2.27 Hz, 1 H) 7.70-7.78 (m, 1 H) 8.29 (s, |
| 23a | 1 H) 9.07 (s, 1 H) 9.47 (s, 1 H) |
| | ¹H NMR (300 MHz, CHLOROFORM-d) δ ppm 1.73 (s, 3 H) 1.95 (dd, J = 14.85, |
| | 4.67 Hz, 1 H) 2.41 (dd, J = 14.85, 5.22 Hz, 1 H) 2.94 (q, J = 5.78 Hz, 1 H) 3.98 (br d, |
| | J = 8.80 Hz, 1 H) 4.38-4.67 (m, 4 H) 7.12 (dd, J = 11.41, 8.94 Hz, 1 H) 7.68 (dd, |
| | J = 7.01, 2.34 Hz, 1 H) 7.72-7.80 (m, 1 H) 8.23 (br s, 1 H) 9.25 (br s, 2 H) 9.33 (br |
| | s, 1 H) 9.55 (s, 1 H) |
| 24a | ¹H NMR (300 MHz, CHLOROFORM-d) δ ppm 1.72 (s, 3 H) 1.94 (dd, J = 14.85, |
| | 4.67 Hz, 1 H) 2.40 (dd, J = 14.92, 5.29 Hz, 1 H) 2.93 (q, J = 5.76 Hz, 1 H) 3.98 (br d, |
| | J = 8.80 Hz, 1 H) 4.36-4.66 (m, 4 H) 7.11 (dd, J = 11.48, 8.87 Hz, 1 H) 7.67 (dd, |
| | J = 7.01, 2.47 Hz, 1 H) 7.76 (dt, J = 8.49, 3.39 Hz, 1 H) 8.21 (dd, J = 8.11, 1.65 Hz, 1 |
| | H) 8.43 (d, J = 8.25 Hz, 1 H) 8.91 (s, 1 H) 9.83 (s, 1 H) |
| 25a | ¹H NMR (400 MHz, CHLOROFORM-d) δ ppm 1.70 (s, 3 H) 1.90 (dd, J = 14.96, |
| | 4.62 Hz, 1 H) 2.47 (dd, J = 14.96, 4.62 Hz, 1 H) 3.14 (dt, J = 7.76, 4.70 Hz, 1 H) 3.98 |
| | (br d, J = 8.80 Hz, 1 H) 4.38-4.49 (m, 1 H) 4.55 (d, J = 8.58 Hz, 1 H) 5.85 (td, |
| | J = 55.84, 4.29 Hz, 1 H) 6.15 (dq, J = 51.06, 1.91 Hz, 2 H) 7.10 (dd, J = 11.66, 8.80 |
| | Hz, 1 H) 7.64 (dd, J = 7.04, 2.86 Hz, 1 H) 7.71 (ddd, J = 8.80, 3.96, 2.86 Hz, 1 H) |
| | 8.28 (d, J = 1.32 Hz, 1 H) 9.07 (d, J = 1.32 Hz, 1 H) 9.46 (s, 1 H) |
| 26a | ¹H NMR (400 MHz, CHLOROFORM-d) δ ppm 1.72 (s, 3 H) 1.91 (dd, J = 15.05, |
| | 4.48 Hz, 1 H) 2.48 (dd, J = 15.05, 4.88 Hz, 1 H) 3.15 (dt, J = 7.83, 4.63 Hz, 1 H) 3.99 |
| | (br d, J = 8.95 Hz, 1 H) 4.40-4.50 (m, 1 H) 4.56 (d, J = 8.54 Hz, 1 H) 5.85 (td, |
| | J = 55.84, 4.27 Hz, 1 H) 7.12 (dd, J = 11.60, 8.75 Hz, 1 H) 7.67-7.76(m, 2 H) 8.22 |
| | (s, 1 H) 9.24 (s, 2 H) 9.32 (d, J = 1.22 Hz, 1 H) 9.54 (s, 1 H) |
| 27a | ¹H NMR (400 MHz, CHLOROFORM-d) δ ppm 1.70 (s, 3 H) 1.90 (dd, J = 14.85, |
| | 4.73 Hz, 1 H) 2.47 (dd, J = 14.96, 4.84 Hz, 1 H) 3.14 (dt, J = 7.59, 4.79 Hz, 1 H) 3.98 |
| | (br d, J = 10.56 Hz, 1 H) 4.40-4.49 (m, 1 H) 4.56 (d, J = 8.58 Hz, 1 H) 5.84 (td, |
| | J = 55.84, 4.29 Hz, 1 H) 7.11 (dd, J = 11.66, 8.80 Hz, 1 H) 7.67 (dd, J = 7.15, 2.75 Hz, |
| | 1 H) 7.73 (ddd, J = 8.80, 4.07, 2.75 Hz, 1 H) 8.21 (dd, J = 8.14, 1.98 Hz, 1 H) 8.42 |
| | (dd, J = 8.14, 0.88 Hz, 1 H) 8.90 (dd, J = 1.98, 0.66 Hz, 1 H) 9.81 (s, 1 H) |
| 31c | ¹H NMR (400 MHz, CHLOROFORM-d) δ ppm 1.34 (d, J = 6.16 Hz, 3 H) 1.68 (s, 3 |
| | H) 1.93 (dd, J = 14.53, 5.06 Hz, 1 H) 2.30 (dd, J = 14.53, 6.16 Hz, 1 H) 2.42 (dt, |
| | J = 7.65, 5.53 Hz, 1 H) 3.88 (dd, J = 9.24, 1.76 Hz, 1 H) 4.27 (quin, J = 6.49 Hz, 1 H) |
| | 4.55 (dd, J = 9.13, 0.99 Hz, 1 H) 6.15 (dq, J = 51.06, 2.05 Hz, 2 H) 7.23 (dt, J = 5.94, |
| | 2.31 Hz, 1 H) 7.88 (ddd, J = 11.44, 6.71, 2.75 Hz, 1 H) 8.29 (d, J = 1.32 Hz, 1 H) 9.06 |
| | (d, J = 1.32 Hz, 1 H) 9.47 (s, 1 H) |
| 32b | ¹H NMR (400 MHz, CHLOROFORM-d) δ ppm 1.34 (d, J = 6.10 Hz, 3 H) 1.70 (s, 3 |
| | H) 1.94 (dd, J = 14.45, 5.09 Hz, 1 H) 2.32 (dd, J = 14.65, 6.10 Hz, 1 H) 2.39-2.45 |
| | (m, 1 H) 3.89 (dd, J = 9.36, 1.22 Hz, 1 H) 4.28 (quin, J = 6.31 Hz, 1 H) 4.56 (d, |
| | J = 9.36 Hz, 1 H) 7.27-7.30(m, 1 H) 7.90 (ddd, J = 11.39, 6.71, 2.64 Hz, 1 H) 8.22 |
| | (s, 1 H) 9.24 (s, 1 H) 9.25 (d, J = 1.30 Hz, 1 H) 9.32 (d, J = 1.34 Hz, 1 H) 9.54 (s, 1 H) |
| 33b | ¹H NMR (400 MHz, CHLOROFORM-d) δ ppm 1.33 (d, J = 5.94 Hz, 3 H) 1.69 (s, 3 |
| | H) 1.93 (dd, J = 14.53, 5.06 Hz, 1 H) 2.28-2.34 (m, 1 H) 2.41 (dt, J = 7.32, 5.69 Hz, |
| | 1 H) 3.88 (dd, J = 9.24, 1.54 Hz, 1 H) 4.27 (quin, J = 6.33 Hz, 1 H) 4.55 (d, J = 9.90 |
| | Hz, 1 H) 7.26-7.28 (m, 1 H) 7.90 (ddd, J = 11.39, 6.66, 2.64 Hz, 1 H) 8.21 (dd, |
| | J = 8.14, 1.98 Hz, 1 H) 8.41 (dd, J = 8.14, 0.88 Hz, 1 H) 8.90 (dd, J = 1.98, 0.88 Hz, 1 |
| | H) 9.82 (s, 1 H) |
| 34a | ¹H NMR (400 MHz, CHLOROFORM-d) δ ppm 1.71 (s, 3 H) 1.85 (dd, J = 15.29, |
| | 4.29 Hz, 1 H) 2.51 (dd, J = 15.29, 3.19 Hz, 1 H) 3.28-3.34 (m, 1 H) 4.05 (dd, |
| | J = 8.36, 1.98 Hz, 1 H) 4.62 (d, J = 8.58 Hz, 1 H) 4.64-4.73 (m, 1 H) 7.09-7.16 (m, |
| | 1 H) 7.66-7.72 (m, 2 H) 8.22 (s, 1 H) 9.23-9.25 (m, 2 H) 9.32 (d, J = 1.32 Hz, 1 H) |
| | 9.54 (s, 1 H) |
| 35a | ¹H NMR (400 MHz, CHLOROFORM-d) δ ppm 1.36 (d, J = 6.12 Hz, 3 H) 1.69 (s, 3 |
| | H) 1.91 (dd, J = 14.69, 4.90 Hz, 1 H) 2.30 (dd, J = 14.69, 5.30 Hz, 1 H) 2.47 (dt, |
| | J = 8.16, 5.10 Hz, 1 H) 3.93 (dd, J = 8.98, 2.04 Hz, 1 H) 4.30 (quin, J = 6.43 Hz, 1 H) |
| | 4.58 (dd, J = 9.18, 1.02 Hz, 1 H) 6.64 (t, J = 71.80 Hz, 1 H) 7.08 (dd, J = 11.63, 8.77 |
| | Hz, 1 H) 7.60 (dd, J = 7.34, 2.86 Hz, 1 H) 7.66 (dd, J = 8.57, 2.45 Hz, 1 H) 7.75 (ddd, |
| | J = 8.98, 4.08, 2.86 Hz, 1 H) 8.31 (d, J = 8.57 Hz, 1 H) 8.46 (d, J = 2.04 Hz, 1 H) 9.79 |
| | (s, 1 H) |
| 36a | ¹H NMR (400 MHz, DMSO-d₆) δ ppm 1.27 (d, J = 5.94 Hz, 3 H) 1.63 (s, 3 H) 1.67 |
| | (dd, J = 14.97, 4.18 Hz, 1 H) 2.22-2.33 (m, 2 H) 3.77 (dd, J = 8.47, 2.75 Hz, 1 H) |
| | 4.19-4.27 (m, 1 H) 4.40 (d, J = 9.46 Hz, 1 H) 7.17 (dd, J = 11.88, 8.80 Hz, 1 H) 7.79 |
| | (dd, J = 7.48, 2.64 Hz, 1 H) 7.86 (ddd, J = 8.75, 4.13, 2.75 Hz, 1 H) 8.41 (d, J = 8.58 |
| | Hz, 1 H) 8.60 (dd, J = 8.47, 2.53 Hz, 1 H) 9.28 (d, J = 2.42 Hz, 1 H) 10.25-10.32 (m, |
| | 1 H) 10.87 (s, 1 H) |
| 37 | ¹H NMR (300 MHz, CHLOROFORM-d) δ ppm 1.67-1.88 (m, 7 H) 2.53 (br d, |
| | J = 15.26 Hz, 1 H) 2.86 (s, 3 H) 3.28-3.36 (m, 1 H) 3.97 (br d, J = 7.97 Hz, 1 H) 4.43 |
| | (br dd, J = 17.32, 7.84 Hz, 1 H) 4.55 (br d, J = 8.40 Hz, 1 H) 7.10 (br t, J = 10.20 Hz, 1 |
| | H) 7.48 (m, J = 6.70 Hz, 1 H) 7.75-7.83 (m, 1 H) 7.96 (br s, 1 H) 8.73 (br s, 1 H) |
| | 9.96 (s, 1 H) |
| 38 | ¹H NMR (300 MHz, CHLOROFORM-d) δ ppm 1.58-1.80 (m, 7 H) 2.45 (dd, |
| | J = 15.12, 3.16 Hz, 1 H) 3.23 (dt, J = 8.14, 3.83 Hz, 1 H) 3.90 (br d, J = 8.11 Hz, 1 H) |
| | 4.34 (ddd, J = 17.60, 8.45, 2.96 Hz, 1 H) 4.48 (d, J = 8.25 Hz, 1 H) 7.03 (dd, J = 11.48, |
| | 8.73 Hz, 1 H) 7.36-7.74 (m, 3 H) 8.26 (br s, 1 H) 8.99 (br s, 1 H) 9.36 (br s, 1 H) |

D. Pharmacological Examples

The compounds provided in the present invention are inhibitors of the beta-site APP-cleaving enzyme 1 (BACE1). Inhibition of BACE1, an aspartic protease, is believed to be relevant for treatment of Alzheimer's Disease (AD). The production and accumulation of beta-amyloid peptides (Abeta) from the beta-amyloid precursor protein

(APP) is believed to play a key role in the onset and progression of AD. Abeta is produced from the amyloid precursor protein (APP) by sequential cleavage at the N- and C-termini of the Abeta domain by beta-site APP-cleaving enzyme and gamma-secretase, respectively.

Compounds of Formula (I), in particular, compounds of Formula (I-a), are expected to have their effect substantially at BACE1 by virtue of their ability to inhibit the enzymatic activity. The behaviour of such inhibitors tested using a biochemical Fluorescence Resonance Energy Transfer (FRET) based assay and a cellular αLisa assay in SKNBE2 cells described below and which are suitable for the identification of such compounds, and more particularly the compounds according to Formula (I), more in particular according to Formula (I-a), are shown in Tables 5-7. The pIC₅₀ values for compounds of Formula (I-b) in the biochemical and cellular assays in Tables 5-7 are reported as measured and suspected to be due to the presence of trace amounts of stereoisomer(s) of Formula (I-a) in the test samples, when the reported pIC₅₀ values are ≥5.

BACE1 Biochemical Fret Based Assay

This assay is a Fluorescence Resonance Energy Transfer Assay (FRET) based assay. The substrate for this assay is an APP derived 13 amino acids peptide that contains the ‘Swedish’ Lys-Met/Asn-Leu mutation of the amyloid precursor protein (APP) beta-site APP-cleaving enzyme cleavage site. This substrate also contains two fluorophores: (7-methoxycoumarin-4-yl) acetic acid (Mca) is a fluorescent donor with excitation wavelength at 320 nm and emission at 405 nm and 2,4-Dinitrophenyl (Dnp) is a proprietary quencher acceptor. The distance between those two groups has been selected so that upon light excitation, the donor fluorescence energy is significantly quenched by the acceptor, through resonance energy transfer. Upon cleavage by BACE1, the fluorophore Mca is separated from the quenching group Dnp, restoring the full fluorescence yield of the donor. The increase in fluorescence is linearly related to the rate of proteolysis.

### Method 1:

Briefly in a 384-well format recombinant BACE1 protein in a final concentration of 1 μg/ml is incubated for 120 minutes at room temperature with 10 μm substrate in incubation buffer (40 mM Citrate buffer pH 5.0, 0.04% PEG, 4% DMSO) in the absence or presence of compound. Next the amount of proteolysis is directly measured by fluorescence measurement at T=0 and T=120 (excitation at 320 nm and emission at 405 nm). Results are expressed in RFU (Relative Fluorescence Units), as difference between T120 and T0.

A best-fit curve is fitted by a minimum sum of squares method to the plot of % Controlmin versus compound concentration. From this an IC₅₀ value (inhibitory concentration causing 50% inhibition of activity) can be obtained.

$$\begin{matrix}LC = & Median of the low control values \\ = & Low control \text{:} Reaction without enzyme\end{matrix}$$

$$\begin{matrix}HC = & Median of the High control values \\ = & High Control \text{:} Reaction with enzyme\end{matrix}$$

$$\% Effect = 100 - [ ( sample - LC ) \text{/} ( HC - LC ) * 100 ]$$

$$\% Control = ( sample \text{/} HC ) * 100$$

$$\% Controlmin = ( sample - LC ) \text{/} ( HC - LC ) * 100$$

The following exemplified compounds were tested essentially as described above and exhibited the following the activity:

TABLE 5

| | Biochemical |
|---|---|
| Co. | FRET based |
| No. | assay pIC₅₀ |
| 1a | 5.05 |
| 1b | 7.89 |
| 2c | 8.73 |
| 2d | 5 |
| 2e | <5 |
| 2f | 7.83 |
| 3c | 9.06 |
| 3d | 5.02 |
| 3e | 5.84 |
| 3f | 8.33 |
| 4b | 8.73 |
| 4c | 6.28 |
| 5b | 7.06 |
| 5c | <5 |
| 6b | 7.89 |
| 6c | 5.12 |
| 7c | 5.97 |
| 7d | 8.79 |
| 7e | 7.68 |
| 7f | 5.74 |
| 8c | 8.75 |
| 8d | 5.37 |
| 9b | 8.28 |
| 9c | 5.76 |
| 10b | 8.25 |
| 10c | 5.01 |
| 11a | 8.53 |
| 12b | 5.27 |
| 12c | 7.95 |
| 13a | 7 |
| 13b | 5.48 |
| 13c | 6.05 |
| 13d | <5 |
| 14 | 8.65 |
| 15a | 8.13 |
| 15b | 5.81 |
| 16a | 8.31 |
| 17a | 8.8 |
| 18a | 7.61 |
| 21b | 7.98 |
| 22a | 8.32 |
| 23a | 7.91 |
| 24a | 8.67 |
| 24b | 7.84 |
| 25a | 8.05 |
| 25b | 7.28 |
| 26a | 8.01 |
| 27a | 8.55 |
| 31a | 7.18 |
| 31b | 5.06 |
| 31c | 7.81 |
| 31d | <5 |
| 32a | 7.72 |
| 32b | 6.51 |
| 33a | 5.12 |
| 33b | 8.26 |
| 33c | 7.45 |
| 33d | 6.15 |
| 34a | 8.2 |
| 34b | 5.99 |
| 35 | 8.17 |
| 35a | 8.25 |
| 35b | 6.13 |
| 36a | 8.61 |
| 36b | 6.54 |
| 37 | 8.72 |
| 38 | 9.02 |

### Cellular αLisa Assay in SKNBE2 Cells

In two αLisa assays the levels of Abeta 1-42 produced and secreted into the medium of human neuroblastoma SKNBE2 cells are quantified. The assay is based on the human neuroblastoma SKNBE2 expressing the wild type Amyloid Precursor Protein (hAPP695). The compounds are diluted and added to these cells, incubated for 18 hours and then measurements of Abeta 1-42 are taken. Abeta 1-42 are measured by sandwich αLisa. αLisa is a sandwich assay using biotinylated antibody AbN/25 attached to streptavidin coated beads and antibody cAb42/26 conjugated acceptor beads for the detection of Abeta 1-42 respectively. In the presence of Abeta 1-42, the beads come into close proximity. The excitation of the donor beads provokes the release of singlet oxygen molecules that trigger a cascade of energy transfer in the acceptor beads, resulting in light emission. Light emission is measured after 1 hour incubation (excitation at 650 nm and emission at 615 nm).

A best-fit curve is fitted by a minimum sum of squares method to the plot of % Controlmin versus compound concentration. From this an IC₅₀ value (inhibitory concentration causing 50% inhibition of activity) can be obtained.

$$\begin{matrix}LC = & Median of the low control values \\ = & Low control \text{:} cells preincubated without compound , without \\ & biotinylated Ab in the α Lisa\end{matrix}$$

$$\begin{matrix}HC = & Median of the High control values \\ = & High Control \text{:} cells preincubated without compound\end{matrix}$$

$$\% Effect = 100 - [ ( sample - LC ) \text{/} ( HC - LC ) * 100 ]$$

$$\% Control = ( sample \text{/} HC ) * 100$$

$$\% Controlmin = ( sample - LC ) \text{/} ( HC - LC ) * 100$$

The following exemplified compounds were tested essentially as described above and exhibited the following the activity:

TABLE 6

| | Cellular |
|---|---|
| | αLisa assay |
| | in SKNBE2 |
| | cells |
| Co. | Abeta 42 |
| No. | pIC₅₀ |
| 1a | 5.12 |
| 1b | 8.47 |
| 2c | 8.32 |
| 2d | <5.05 |
| 2e | <5.05 |
| 2f | 7.47 |
| 3c | 8.52 |
| 3d | <5.05 |
| 3e | 5.34 |
| 3f | 8.1 |
| 4b | 8.9 |
| 4c | 6.22 |
| 5b | 7.46 |
| 5c | <5.05 |
| 6b | 8.12 |
| 6c | 5.14 |
| 7c | 5.32 |
| 7d | 8.02 |
| 7e | 7.33 |
| 7f | 5.43 |
| 8c | 8.17 |
| 8d | 5.1 |
| 9b | 8.74 |
| 9c | 5.8 |
| 10b | 8.23 |
| 10c | 5.29 |
| 11a | 8.58 |
| 11b | 5.9 |
| 12b | 5.45 |
| 12c | 8.13 |
| 13a | 7.19 |
| 13b | 5.75 |
| 13c | 6.21 |
| 13d | <5.05 |
| 14 | 8.16 |
| 15a | 8.19 |
| 15b | 5.62 |
| 16a | 7.78 |
| 17a | 8.67 |
| 18a | 6.86 |
| 21a | 8.62 |
| 21b | 7.71 |
| 22a | 7.88 |
| 23a | 7.59 |
| 24a | 8.54 |
| 24b | 7.5 |
| 25a | 7.74 |
| 25b | 7.13 |
| 26a | 7.5 |
| 27a | 8.2 |
| 31a | 7.15 |
| 31b | <5.05 |
| 31c | 7.57 |
| 31d | <5.05 |
| 32a | 7.51 |
| 32b | 5.97 |
| 33a | 5.14 |
| 33b | 7.81 |
| 33c | 7.37 |
| 33d | 5.78 |
| 34a | 7.57 |
| 34b | 5.31 |
| 35 | 8 |
| 35a | 8.32 |
| 35b | 6.11 |
| 36a | 7.63 |
| 36b | 5.83 |
| 37 | 8.75 |
| 38 | 8.42 |

### BACE2 Biochemical FRET Based Assay

This assay is a Fluorescence Resonance Energy Transfer Assay (FRET) based assay. The substrate for this assay contains the ‘Swedish’ Lys-Met/Asn-Leu mutation of the amyloid precursor protein (APP) beta-site APP-cleaving enzyme cleavage site. This substrate also contains two fluorophores: (7-methoxycoumarin-4-yl) acetic acid (Mca) is a fluorescent donor with excitation wavelength at 320 nm and emission at 405 nm and 2,4-Dinitrophenyl (Dnp) is a proprietary quencher acceptor. The distance between those two groups has been selected so that upon light excitation, the donor fluorescence energy is significantly quenched by the acceptor, through resonance energy transfer. Upon cleavage by the beta-site APP-cleaving enzyme, the fluorophore Mca is separated from the quenching group Dnp, restoring the full fluorescence yield of the donor. The increase in fluorescence is linearly related to the rate of proteolysis.

Briefly in a 384-well format recombinant BACE2 protein in a final concentration of 0.4 μg/ml is incubated for 450 minutes at room temperature with 10 μM substrate in incubation buffer (50 mM Citrate buffer pH 5.0, 0.05% PEG, no DMSO) in the absence or presence of compound. Next the amount of proteolysis is directly measured by fluorescence measurement at T=0 and T=450 (excitation at 320 nm and emission at 405 nm). Results are expressed in RFU (Relative Fluorescence Units), as difference between T450 and T0.

A best-fit curve is fitted by a minimum sum of squares method to the plot of % Controlmin versus compound concentration. From this an IC₅₀ value (inhibitory concentration causing 50% inhibition of activity) can be obtained.

$$\begin{matrix}LC = & Median of the low control values \\ = & Low control \text{:} Reaction without enzyme\end{matrix}$$

$$\begin{matrix}HC = & Median of the High control values \\ = & High Control \text{:} Reaction with enzyme\end{matrix}$$

$$\% Effect = 100 - [ ( sample - LC ) \text{/} ( HC - LC ) * 100 ]$$

$$\% Control = ( sample \text{/} HC ) * 100$$

$$\% Controlmin = ( sample - LC ) \text{/} ( HC - LC ) * 100$$

The following exemplified compounds were tested essentially as described above and exhibited the following the activity:

TABLE 7

| | Biochemical |
|---|---|
| Co. | FRET based |
| No. | assay pIC₅₀ |
| 1a | <5 |
| 1b | 7.21 |
| 2c | 8.3 |
| 2d | <5 |
| 2e | <5 |
| 2f | 7.35 |
| 3c | 7.97 |
| 3d | <5 |
| 3e | <5 |
| 3f | 6.88 |
| 4b | 7.84 |
| 4c | 5.27 |
| 5b | 6.17 |
| 5c | <5 |
| 6b | 6.68 |
| 6c | <5 |
| 7c | <5 |
| 7d | 7.25 |
| 7e | 6.24 |
| 7f | <5 |
| 8c | 8.46 |
| 8d | 5.08 |
| 9b | 7.19 |
| 9c | <5 |
| 10b | 8.28 |
| 10c | <5 |
| 11a | 6.88 |
| 12c | 7.26 |
| 13a | 5.9 |
| 13b | <5 |
| 13c | 5.41 |
| 13d | <5 |
| 14 | 7.09 |
| 15a | 6.8 |
| 15b | <5 |
| 16a | 6.44 |
| 17a | 7.67 |
| 18a | 5.92 |
| 21b | 6.15 |
| 22a | 7.13 |
| 23a | 6.45 |
| 24b | 6.8 |
| 25a | 6.85 |
| 25b | 6.05 |
| 26a | 6.98 |
| 27a | 7.44 |
| 31a | 5.62 |
| 31b | <5 |
| 31c | 6.8 |
| 31d | <5 |
| 32a | 6.46 |
| 32b | 5.02 |
| 33a | <5 |
| 33b | 7.35 |
| 33c | 6.33 |
| 33d | 5.05 |
| 34a | 6.58 |
| 34b | <5 |
| 35 | 7.07 |
| 35a | 7.08 |
| 35b | <5 |
| 36a | 6 |
| 36b | <5 |

Pharmacology in the Beagle Dog

Test compounds were tested to evaluate the effect on the beta-amyloid profile in cerebrospinal fluid (CSF) of dogs after a single dose, in combination with pharmacokinetic (PK) follow up and limited safety evaluation. For each of compound 4b, 3c, 7d, or 14, four beagle dogs (2 males, 2 females) were dosed with vehicle (1 ml/kg of an aqueous solution of 20% cyclodextrin) and 12, 8, 4 and 4 beagle dogs (2 males and 2 females per dosage group), were dosed with test compounds as follows:

| Co. | |
|---|---|
| No. | Dosage |
| 4b | 0.16, 0.31 and 1.25 mg/kg in 0.16, 0.31 and 1.25 mg/ml of an aqueous 20% |
| | cyclodextrin solution, on an empty stomach |
| 3c | 0.08 and 0.31 mg/kg in 0.08 and 0.31 mg/ml of an aqueous 20% cyclodextrin |
| | solution, on an empty stomach |
| 7d | 0.31 mg/kg in 0.31 mg/ml of an aqueous 20% cyclodextrin solution, on an |
| | empty stomach |
| 14 | 0.63 mg/kg in 0.63 mg/ml of an aqueous 20% cyclodextrin solution, on an |
| | empty stomach |

CSF was taken in conscious animals directly from the lateral ventricle via a cannula which was screwed in the skull and covered with subcutaneous tissue and skin, before and at 4, 8, 25 and 49 hours after dosing. Eight hours after dosing the animals got access to their regular meal for 30 minutes. Blood was taken for PK follow up (0.5, 1, 2, 4, 8, 25 and 49 hours) and serum samples for biochemistry were taken before and at 8 and 25 h after dosing. The CSF samples were used for measurement of Abeta 1-42. The results are summarized in Table 8 below:

TABLE 8

| | % Mean | % Mean | % Mean | | |
|---|---|---|---|---|---|
| | decrease | decrease | decrease | | |
| | in Abeta | in Abeta | in Abeta | | |
| | 1-42 at | 1-42 at | 1-42 at | | |
| | 8 h post | 25 h post | 49 h post | | |
| | dosing | dosing | dosing | | EC₅₀ |
| | compared | compared | compared | Dose | Aβ₄₂ |
| Co. | to own | to own | to own | (mg/ | (ng/ |
| No. | baseline | baseline | baseline | kg) | mL) |
| 4b | 42 | 21 | NR | 0.16 | 24 |
| | 54 | 53 | NR | 0.31 | |
| | 80 | 71 | 34 | 1.25 | |
| 3c | 29 | 27 | NR | 0.08 | 19 |
| | 64 | 62 | 50 | 0.31 | |
| 7d | 42 | NR | — | 0.31 | — |
| 14 | 32 | NR | NR | 0.63 | — |

NR means % decrease < 20

Observations: compound 4b—one female dog dosed at 1.25 mg/kg had slight diarrhea, 8 h post dosing; compound 3c—one female dog dosed at 0.08 mg/kg had moderate diarrhea 8 h post dosing; compound 7d—one female dog had a 6 fold ALT liver enzyme increase and experienced slight tremors and head shaking 49 h post dosing, therefore no CSF sample was taken at this time point; compound 14—one dog experienced emesis 2 h post dosing.

No other observations or changes in biochemical parameters were observed in any other animals.

1. A compound of Formula (I-a) 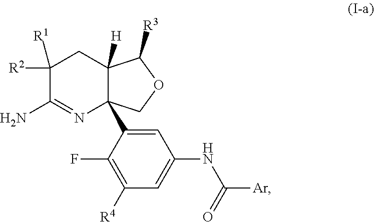
or a tautomer or a stereoisomeric form thereof, wherein
R¹ is selected from the group consisting of hydrogen, C₁₋₄alkyl, monohalo-C₁₋₄alkyl, and polyhalo-C₁₋₄alkyl;
R² is selected from the group consisting of hydrogen, cyano, C₁₋₄alkyloxy, —SO₂C₁₋₄alkyl, —SO₂cyclopropyl, and —SO(NCH₃)CH₃;
R³ is selected from the group consisting of hydrogen, C₁₋₄alkyl optionally substituted with 1, 2 or 3 fluoro substituents, and cyclopropyl optionally substituted with 1 or 2 fluoro substituents;
R⁴ is hydrogen or fluoro;
Ar is homoaryl or heteroaryl;
wherein homoaryl is phenyl, or phenyl substituted with one, two or three substituents each independently selected from the group consisting of halo, cyano, C₁₋₄alkyl,
C₁₋₄alkyloxy, monohalo-C₁₋₄alkyl, polyhalo-C₁₋₄alkyl, monohalo-C₁₋₄alkyloxy, and polyhalo-C₁₋₄alkyloxy;
heteroaryl is selected from the group consisting of pyridyl, pyrimidinyl, pyrazinyl, pyridazinyl, furanyl, thienyl, pyrrolyl, pyrazolyl, imidazolyl, triazolyl, tetrazolyl, thiazolyl, isothiazolyl, thiadiazolyl, oxazolyl, isoxazolyl, and oxadiazolyl, each optionally substituted with one, two or three substituents each independently selected from the group consisting of halo, cyano, C₁₋₄alkyl, C₂₋₄alkynyl, C₁₋₄alkyloxy, monohalo-C₁₋₄alkyl, polyhalo-C₁₋₄alkyl, monohalo-C₁₋₄alkyloxy,
polyhalo-C₁₋₄alkyloxy, C₁₋₄alkyloxyC₁₋₄alkyloxy and triazolyl;
or a pharmaceutically acceptable acid addition salt thereof.

2. The compound according to claim 1 wherein
R¹ is selected from the group consisting of hydrogen, C₁₋₄alkyl, monohalo-C₁₋₄alkyl, and polyhalo-C₁₋₄alkyl;
R² is selected from the group consisting of hydrogen, cyano, C₁₋₄alkyloxy, —SO₂C₁₋₄alkyl, —SO₂cyclopropyl, and —SO(NCH₃)CH₃;
R³ is selected from the group consisting of hydrogen; C₁₋₄alkyl optionally substituted with 1-3 fluoro substituents, and cyclopropyl optionally substituted with 1 or 2 fluoro substituents;
R⁴ is hydrogen or fluoro;
Ar is homoaryl or heteroaryl;
wherein homoaryl is phenyl, or phenyl substituted with one, two or three substituents each independently selected from the group consisting of halo, cyano, C₁₋₄alkyl, C₁₋₄alkyloxy, monohalo-C₁₋₄alkyl, polyhalo-C₁₋₄alkyl, monohalo-C₁₋₄alkyloxy, and polyhalo-C₁₋₄alkyloxy;
heteroaryl is selected from the group consisting of pyridyl, pyrimidinyl, pyrazinyl, pyridazinyl, furanyl, thienyl, pyrrolyl, pyrazolyl, imidazolyl, triazolyl, tetrazolyl, thiazolyl, isothiazolyl, thiadiazolyl, oxazolyl, isoxazolyl, and oxadiazolyl, each optionally substituted with one, two or three substituents each independently selected from the group consisting of halo, cyano, C₁₋₄alkyl, C₂₋₄alkynyl, C₁₋₄alkyloxy, monohalo-C₁₋₄alkyl, polyhalo-C₁₋₄alkyl, monohalo-C₁₋₄alkyloxy,
polyhalo-C₁₋₄alkyloxy, C₁₋₄alkyloxyC₁₋₄alkyloxy and triazolyl
or a pharmaceutically acceptable addition salt or a solvate thereof.

3. The compound according to claim 2, wherein R¹ is selected from the group consisting of hydrogen, C₁₋₄alkyl, monohalo-C₁₋₄alkyl, and polyhalo-C₁₋₄alkyl;
R² is selected from the group consisting of hydrogen, cyano, C₁₋₄alkyloxy, —SO₂C₁₋₄alkyl, —SO₂cyclopropyl, and —SO(NCH₃)CH₃;
R³ is selected from the group consisting of hydrogen and C₁₋₄alkyl optionally substituted with 1-3 fluoro substituents;
R⁴ is hydrogen or fluoro;
Ar is homoaryl or heteroaryl;
wherein homoaryl is phenyl, or phenyl substituted with one, two or three substituents each independently selected from the group consisting of halo, cyano, C₁₋₄alkyl, C₁₋₄alkyloxy, monohalo-C₁₋₄alkyl, polyhalo-C₁₋₄alkyl, monohalo-C₁₋₄alkyloxy, and polyhalo-C₁₋₄alkyloxy;
heteroaryl is selected from the group consisting of pyridyl, pyrimidinyl, pyrazinyl, pyridazinyl, furanyl, thienyl, pyrrolyl, pyrazolyl, imidazolyl, triazolyl, tetrazolyl, thiazolyl, isothiazolyl, thiadiazolyl, oxazolyl, isoxazolyl, and oxadiazolyl, each optionally substituted with one, two or three substituents each independently selected from the group consisting of halo, cyano, C₁₋₄alkyl, C₂₋₄alkynyl, C₁₋₄alkyloxy, monohalo-C₁₋₄alkyl, polyhalo-C₁₋₄alkyl, monohalo-C₁₋₄alkyloxy, polyhalo-C₁₋₄alkyloxy, and C₁₋₄alkyloxyC₁₋₄alkyloxy.

4. The compound according claim 3 wherein
R¹ is selected from the group consisting of hydrogen, and C₁₋₄alkyl;
R² is selected from the group consisting of hydrogen, cyano, and —SO₂C₁₋₄alkyl;
R³ is selected from the group consisting of hydrogen, and C₁₋₃alkyl optionally substituted with 1-3 fluoro substituents;
R⁴ is hydrogen or fluoro; and
Ar is selected from the group consisting of pyridyl, pyrimidinyl, pyrazinyl, and pyridazinyl, each optionally substituted with one, two or three substituents each independently selected from the group consisting of halo, cyano, C₁₋₄alkyl, C₁₋₄alkyloxy, monohalo-C₁₋₄alkyl, polyhalo-C₁₋₄alkyl, monohalo-C₁₋₄alkyloxy, and polyhalo-C₁₋₄alkyloxy.

5. The compound according to claim 4, wherein Ar is pyridyl or pyrazinyl, each optionally substituted with one, two or three substituents each independently selected from the group consisting of cyano, mono-halo-C₁₋₄alkyloxy, and polyhalo-C₁₋₄alkyloxy.

6. The compound according to claim 5, wherein the compound is 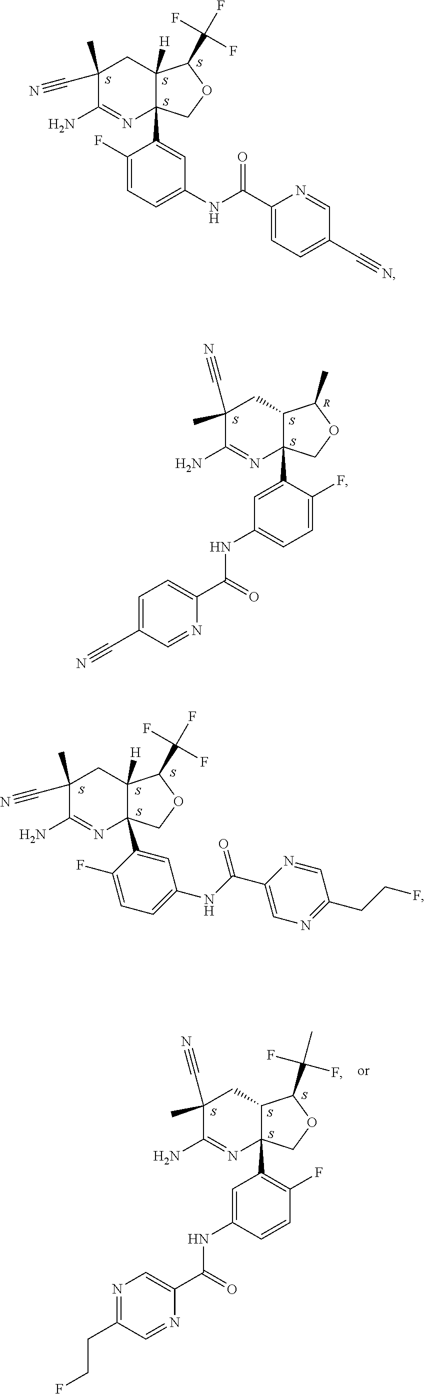 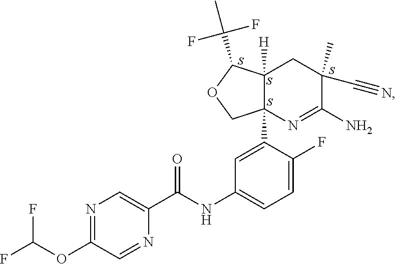
or a pharmaceutically acceptable addition salt thereof.

7. A pharmaceutical composition comprising a therapeutically effective amount of a compound according to claim 1 and a pharmaceutically acceptable carrier.

8. A process for preparing a pharmaceutical composition comprising mixing a pharmaceutically acceptable carrier with a therapeutically effective amount of a compound according to claim 1.

9. (canceled)

10. (canceled)

11. A method of treating a disorder selected from the group consisting of Alzheimer's disease, mild cognitive impairment, preclinical Alzheimer's disease, senility, dementia, dementia with Lewy bodies, Down's syndrome, dementia associated with stroke, dementia associated with Parkinson's disease, and dementia associated with beta-amyloid comprising administering to a subject in need thereof, a therapeutically effective amount of a compound according to claim.

12. A method for modulating beta-site amyloid cleaving enzyme activity, comprising administering to a subject in need thereof, a therapeutically effective amount of a compound according to claim 1.

13. (canceled)

14. A compound according to claim 1 wherein Ar is 1,2,4-triazol-1-yl.

15. A compound according to claim 2 wherein Ar is 1,2,4-triazol-1-yl.

16. A method of treating a disorder selected from the group consisting of Alzheimer's disease, mild cognitive impairment, preclinical Alzheimer's disease, senility, dementia, dementia with Lewy bodies, Down's syndrome, dementia associated with stroke, dementia associated with Parkinson's disease, and dementia associated with beta-amyloid comprising administering to a subject in need thereof, a therapeutically effective amount of the pharmaceutical composition according to claim 7.

17. A method for modulating beta-site amyloid cleaving enzyme activity, comprising administering to a subject in need thereof, a therapeutically effective amount of the pharmaceutical composition according to claim 7.
